(19) United States
(12) Patent Application Publication
(10) Pub. No.: US 2026/0234161 A1
(43) Pub. Date: Aug. 13, 2026

(54) RIFABUTIN ANALOGS FOR THE TREATMENT OF BACTERIAL DISEASES

(71) Applicant: BIOVERSYS AG, Basel (CH)

(72) Inventors: Kevin ANTRAYGUES, Loos (FR); Marilyne BOUROTTE, Perenchies (FR); Glenn E. DALE, Basel (CH); Olivier DEFERT, Lille (FR); Aurore DRENEAU, Lille (FR); Marc GITZINGER, Basel (CH); Sergio LOCIURO, Basel (CH); Mathieu MAINGOT, Lille (FR); Alessia MICHELOTTI, La Madeleine (FR); Vincent TREBOSC, Kembs (FR); Nicolas WILLAND, Lille (FR); Nawaz KHAN, Basel (CH); Line HOFMANN, Lille (FR); Marija MILJKOVIC, Basel (CH)

(21) Appl. No.: 19/154,551

(22) PCT Filed: Feb. 7, 2024
(86) PCT No.: PCT/EP2024/052975

§ 371 Date: Aug. 7, 2025

(30) Foreign Application Priority Data
Feb. 10, 2023 (EP) 23156170.5

(51) Int. Cl. C07D 498/22; A61K 31/438; A61K 31/444; A61K 31/4545; A61K 31/4725; A61K 31/496; A61K 31/4985; A61K 31/499; A61K 31/4995; A61K 31/5377; A61K 31/5386; A61K 31/551; A61P 31/04; C07D 519/00

(52) U.S. Cl. CPC C07D 498/22; A61K 31/438; A61K 31/444; A61K 31/4545; A61K 31/4725; A61K 31/496; A61K 31/4985; A61K 31/499; A61K 31/4995; A61K 31/5377; A61K 31/5386; A61K 31/551; A61P 31/04; C07D 519/00

(57) ABSTRACT

The present invention relates to compounds and pharmaceutical compositions comprising the same for the treatment, amelioration and/or prevention of disease. In some embodiments, the disease is a bacterial infection. In some embodiments, the bacterial infection is caused by one or more bacterium belonging to a genus of non-tuberculous Mycobacteria, preferably M. abscessus.

The present invention relates to compounds and pharmaceutical compositions comprising the same for the treatment, amelioration and/or prevention of disease. In some embodiments, the disease is an infection, preferably a bacterial infection. In some embodiments, the bacterium belongs to the genus or species Acinetobacter spp., Clostridium spp., Enterococcus spp., Hemophilus spp., Legionella spp., Mycobacterium spp., Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii.

In some embodiments the infection is caused by A. baumannii, and/or S. aureus, and/or a genus of non-tuberculous Mycobacteria (NTM), preferably M. abscessus.

### RELATED ART

Rifamycins such as rifabutin are known antibiotics with activity against a broad spectrum of pathogens such as Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and Toxoplasma gondii (Kunin, Clin. Infect. Dis., 1996; Farr and Mandell, Med. Clin. North. Am., 1982; Thornsberry et al., Rev. Infect. Dis., 1983; Hoover et al., Diagn. Microbiol. Infect. Dis., 1993; Kerry et al., J. Antimicrob. Chemother., 1975).

Rifabutin has been recently shown to have potent in vitro and in vivo activity against Mycobacterium abscessus (Aziz et al., Antimicrob. Agents Chemother., 2017; Dick et al., Antimicrob. Agents Chemother., 2020) and Acinetobacter baumannii (Luna et al., Nat. Microbiol., 2020; Trebosc et al., Drug Discov. Today, 2021; Trebosc et al., J. Antimicrob. Chemother., 2020). However, there remains a need for more effective rifamycins for the treatment of bacterial infections such as M. abscessus and A. baumannii infections.

### SUMMARY OF THE INVENTION

In one aspect, the present invention provides a compound of Formula I or a pharmaceutically acceptable salt, tautomer, solvate, hydrate or enantiomer thereof:

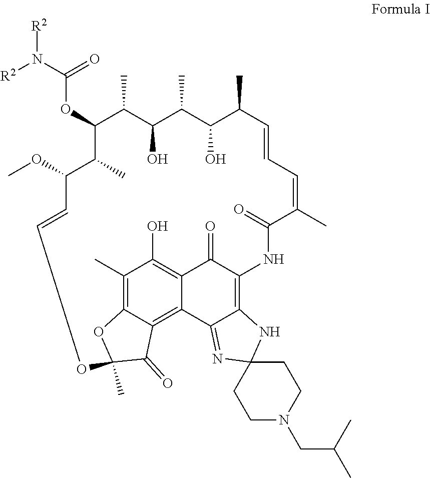

wherein:
R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —CN, —C₁-C₆ alkoxy, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In one aspect, the present invention provides a compound of Formula I or a pharmaceutically acceptable salt, tautomer, solvate, hydrate or enantiomer thereof:

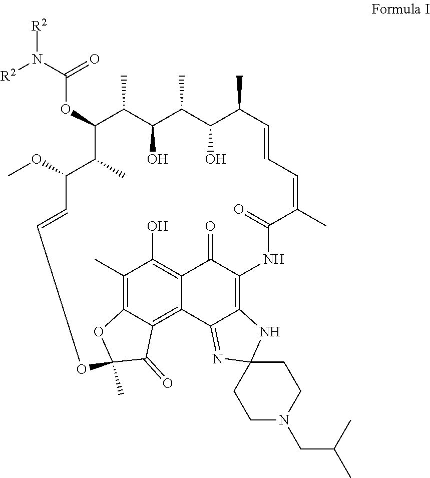

wherein:
R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In one aspect, the present invention provides a pharmaceutical composition comprising a compound as described herein, or a pharmaceutically acceptable salt thereof, and a pharmaceutically acceptable carrier. In some preferred embodiments, the pharmaceutical composition is effective for treating a disease, preferably an infection, more preferably a bacterial infection. In some preferred embodiments, the bacterial infection is caused by one or more bacterium belonging to the genus Acinetobacter, Staphylococcus, and/or Mycobacteria.

In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a compound of Formula I or a pharmaceutically acceptable salt thereof as described herein for use as a medicament.

In one aspect, the present invention provides a compound of Formula I or a pharmaceutically acceptable salt thereof as described herein for use in a method for treating a disease, preferably an infection, more preferably a bacterial infection in a subject. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a use of a compound or pharmaceutical composition comprising a compound as described herein in the manufacture of a medicament for treating a disease, preferably an infection, more preferably a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a method of treating a disease, preferably an infection, more preferably a bacterial infection in a subject in need thereof, comprising administering to the subject a therapeutically effective amount of a compound as described herein or a pharmaceutically acceptable salt thereof. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

The present invention provides rifabutin analogs that are modified at the C25 position and pharmaceutical compositions thereof. The inventive compounds exhibit broad antibacterial activity against a wide array of bacterial species, and thus maintain the broad antibacterial activity characteristic of the rifamycin class of antibiotics. Additional features and advantages of the present technology will be apparent to one of skill in the art upon reading the Detailed Description, below.

### DETAILED DESCRIPTION OF THE INVENTION

The present invention provides analogs of rifabutin that are effective in treating bacterial infections, preferably bacterial infections caused by one or more bacterium belonging to a genus of non-tuberculous Mycobacteria, preferably M. abscessus.

The details of the technology are set forth in the accompanying description below. Although methods and materials similar or equivalent to those described herein can be used in the practice or testing of the present technology, illustrative methods and materials are now described. Other features, objects, and advantages of the invention will be apparent from the description and from the claims.

### Definitions

Unless defined otherwise, all technical and scientific terms used herein have the same meanings as commonly understood by one of ordinary skill in the art to which this invention belongs.

The articles “a” and “an” are used in this disclosure to refer to one or more than one (i.e., to at least one) of the grammatical object of the article unless the context clearly dictates otherwise. By way of example, “an element” means one element or more than one element.

The term “and/or” is used in this disclosure to mean either “and” or “or” unless indicated otherwise.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. The term “alkyl” refers to a straight or branched chain saturated hydrocarbon. C₁-C₆ alkyl groups contain 1 to 6 carbon atoms. Examples of a —C₁-C₆ alkyl group include, but are not limited to, methyl, ethyl, propyl, butyl, pentyl, isopropyl, isobutyl, sec-butyl and tert-butyl, isopentyl and neopentyl.

The terms “alkylene” or “alkylenyl,” as used herein, refer to a straight or branched hydrocarbon chain bi-radical derived from alkyl, as defined herein, wherein one hydrogen of said alkyl is cleaved off generating the second radical of said alkylene. Examples of alkylene are, by way of illustration, —CH₂—, —CH₂—CH₂—, —CH(CH₃)—, —CH₂—CH₂—CH₂—, —CH(CH₃)—CH₂—, or —CH(CH₂CH₃)—.

“C₁-C₆ alkoxy”, as used herein, refers to straight chain or branched saturated —O—C₁-C₆ hydrocarbon which may be, for example, methoxy, ethoxy, n-propoxy, isopropoxy, n-butoxy, isobutoxy, sec-butoxy, tert-butoxy, straight or branched pentoxy, straight or branched hexyloxy, straight or branched heptyloxy, or straight or branched octyloxy. Preferably, C₁-C₆ alkoxy is C₁-C₄ alkoxy. A “C₁-C₆ alkoxy” group can also be represented as “—O—C₁-C₆ alkyl.”

The term “cycloalkyl” means monocyclic or bicyclic saturated carbon rings containing 3-10 carbon atoms. Examples of cycloalkyl groups include, without limitations, cyclopropyl, cyclobutyl, cyclopentyl, cyclohexyl, cycloheptyl, cyclooctyl, cyclononyl, cyclodecyl, octahydro-1H-indene, and decalin.

The term “aryl” refers to cyclic, aromatic hydrocarbon groups that have 1 to 2 aromatic rings, including monocyclic or bicyclic groups such as phenyl or naphthyl. A C₆-C₁₀ aryl group contains between 6 and 10 carbon atoms. When containing two aromatic rings (bicyclic, etc.), the aromatic rings of the aryl group may fused (e.g., naphthyl). The aryl group may be optionally substituted by one or more substituents, e.g., 1 to 5 substituents, at any point of attachment. Exemplary substituents include, but are not limited to, —H, OH, or -halogen. The substituents (e.g., alkyl groups) can themselves be optionally substituted.

Unless otherwise specifically defined, “heteroaryl” means a monovalent monocyclic or bicyclic aromatic radical of 5 to 15 ring atoms containing one or more ring heteroatoms selected from N, S, P, and O, the remaining ring atoms being C, and wherein each ring of the heteroaryl group comprises delocalized π electrons (aromaticity) shared among the ring atoms. Preferably the heteroatom is selected from N, S, and O, more preferably N and O. Preferably the heteroaryl group is a5-10 membered heteroaryl group containing between 5 and 10 ring atoms. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, quinolyl, isothiazolyl, thiazolyl, thiadiazole, indazole, benzimidazolyl, 1,3-dihydro-2H-benzimidazol-2-one, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, benzofuran, quinolinyl, isoquinolinyl, 1,6-naphthyridinyl, thieno[2,3-b]pyrazinyl, quinazolinyl, tetrazolo[1,5-a]pyridinyl, [1,2,4]triazolo[4,3-a]pyridinyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, pyridin-2-one, furo[3,2-c]pyridinyl, furo[2,3-c]pyridinyl, benzooxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, thiazolo[5,4-d]thiazolyl, imidazo[2,1-b][1,3,4]thiadiazolyl, and thieno[2,3-b]pyrrolyl.

The terms “heterocyclyl” or “heterocycloalkyl” or “heterocycle” refer to monocyclic or polycyclic saturated or partially saturated 3 to 15-membered rings containing carbon and heteroatoms taken from O, N, and S (preferably O and N) and wherein at least one ring does not comprise delocalized π electrons (aromaticity) shared among the ring carbon or heteroatoms. A 3-10 membered heterocycloalkyl group contains between 3 and 10 atoms.

Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, piperidinyl, morpholinyl, thiomorpholinyl, thiomorpholinyl S-oxide, thiomorpholinyl S-dioxide, piperazinyl, azepinyl, oxepinyl, [1,4]diazepane, [1,2]diazepane, decahydro-[1,6]naphthyridine diazepinyl, octahydro-pyrrolo[1,2-a]pyrazinyl, octahydro-pyrrolo[3,2-c]pyridinyl, and 2,5-diaza-bicyclo[2.2.1]heptanyl.

A heterocyclyl or heterocycloalkyl ring can also be fused or bridged, e.g., can be a bicyclic or tricyclic ring. Furthermore, when containing two or more fused rings, the heterocycloalkyl groups herein defined can have a saturated or partially saturated ring fused with an aromatic and/or heteroaromatic ring. Exemplary ring systems of such heterocycle-aryl or heterocycle-heteroaryl groups include indolinyl, indolinone, dihydrobenzo thiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, 2,3,4,5-tetrahydro-1H-pyrido[4,3-b]indole, and 5,6,7,8-tetrahydro-imidazo[1,2-a]pyrazine.

A heterocyclyl or heterocycloalkyl ring can also be a spirocyclic heterocycle or spiroheterocycle. As used herein a spirocyclic heterocycle or spiroheterocycle is understood to mean a bicyclic or multicyclic ring system in which at least two rings are connected through a single atom, and wherein at least one of the rings is a heterocycle (e.g., at least one of the rings is, e.g., furanyl, morpholinyl, or piperadinyl). One or both of the rings in a spiroheterocycle can be can be fused to one or more additional carbocyclic, heterocyclic, aromatic, or heteroaromatic ring to form, e.g., a tricyclic ring system in which two of the rings are connected through a single atom. Exemplary spirocyclic heterocycles include but are not limited to 1,3,8-triaza-spiro[4.5]decane, 2,8-diaza-spiro[4.5]decane, 2,9-diaza-spiro[5.5]undecane, 3,9-diaza-spiro[5.5]undecane, 1-oxa-4,9-diaza-spiro[5.5]undecane, and 5-oxa-2,8-diaza-spiro[3.5]nonane.

As used herein, the term “halo” or “halogen” means fluoro (F), chloro (Cl), bromo (Br), or iodo (I).

The term “oxo” refers to a carbonyl functional group composing a carbon atom double-bonded to an oxygen atom. It can be abbreviated herein as “oxo”, as C(O), or as C═O.

The term “CN” refers to the cyano group, also known as a nitrile group.

The invention also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “stereoisomers” refers to the set of compounds which have the same number and type of atoms and share the same bond connectivity between those atoms, but differ in three-dimensional structure. The term “stereoisomer” refers to any member of this set of compounds.

The term “diastereomers” refers to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers. In some cases these diastereomers were separated and in other cases a wavy bond is used to indicate the structural element where configuration is variable.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as “hydrates.” Hydrates include compositions containing stoichiometric amounts of water, as well as compositions containing variable amounts of water.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds of the Invention

In one aspect, the present invention provides a compound of Formula I or a pharmaceutically acceptable salt, tautomer, solvate, hydrate or enantiomer thereof:

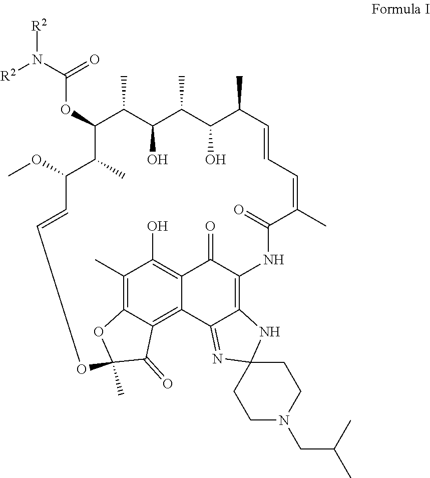

wherein:
R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —CN, —C₁-C₆ alkoxy, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In one aspect, the present invention provides a compound of Formula I or a pharmaceutically acceptable salt, tautomer, solvate, hydrate or enantiomer thereof:

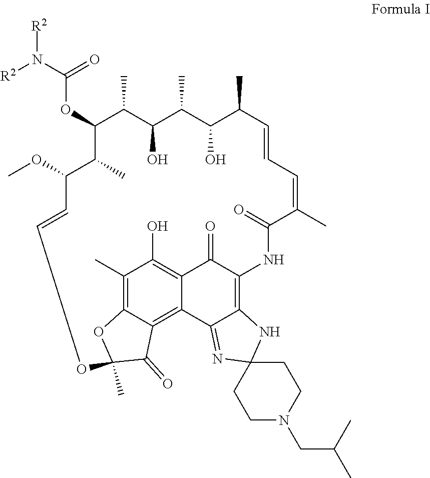

wherein:
R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In some embodiments, R¹ and R² are each independently selected from —H, —C₁-C₆ alkyl, —C₃-C₆ cycloalkyl, C₁-C₆ alkylene-C₃-C₆ cycloalkyl, -5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, and —C₁-C₆ alkylene-(5- to 6-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —CN, —OC₁-C₆ alkyl, —C₁-C₆ alkyl, —C₃-C₆ cycloalkyl, —C₁-C₆ alkylene-C₃-C₆ cycloalkyl, 5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, or —C₁-C₆ alkylene-(5- to 6-membered heteroaryl), wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₆ cycloalkyl, —C₁-C₆ alkylene-C₃-C₆ cycloalkyl, 5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, or —C₁-C₆ alkylene-(5- to 6-membered heteroaryl), wherein each alkylene is optionally substituted with one or more oxo, and wherein each alkyl, cycloalkyl, heterocycloalkyl, aryl or heteroaryl is optionally substituted with one or more R;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₆ cycloalkyl, 5- to 6-membered heterocycloalkyl, phenyl, or 5- to 6-membered heteroaryl; and
R⁸ is independently, at each occurrence, —OH, halogen, or —C₁-C₆ alkyl.

In some embodiments, R¹ and R² are each independently selected from —H, —C₁-C₆ alkyl, —C₃-C₆ cycloalkyl, C₁-C₆ alkylene-C₃-C₆ cycloalkyl, -5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, and —C₁-C₆ alkylene-(5- to 6-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₆ alkyl, —C₃-C₆ cycloalkyl, —C₁-C₆ alkylene-C₃-C₆ cycloalkyl, 5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, or —C₁-C₆ alkylene-(5- to 6-membered heteroaryl), wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₆ cycloalkyl, —C₁-C₆ alkylene-C₃-C₆ cycloalkyl, 5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, or —C₁-C₆ alkylene-(5- to 6-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₆ cycloalkyl, 5- to 6-membered heterocycloalkyl, phenyl, or 5- to 6-membered heteroaryl; and
R⁸ is independently, at each occurrence, —OH, halogen, or —C₁-C₆ alkyl.

In some embodiments, R¹ and R² are each independently selected from —H, —C₁-C₄ alkyl, —C₃-C₁₀ cycloalkyl, C₁-C₄ alkylene-C₃-C₁₀ cycloalkyl, -5- to 15-membered heterocycloalkyl, and —C₁-C₄ alkylene-(5- to 15-membered heterocycloalkyl), wherein each alkyl, cycloalkyl, or heterocycloalkyl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄alkylene-(5- to 10-membered heteroaryl); wherein each cycloalkyl, heterocycloalkyl, aryl, or heteroaryl is optionally substituted with one or more —OH, halogen, or C₁-C₄ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 10-membered heterocycloalkyl, C₆-C₁₀ aryl, or 5- to 10-membered heteroaryl; and
R⁸ is independently, at each occurrence, —OH, halogen, or —C₁-C₄ alkyl.

In some embodiments, R¹ and R² are each independently selected from —H, C₁-C₄ alkyl, C₃-C₆ cycloalkyl, -5- to 15-membered heterocycloalkyl, and —C₁-C₄ alkylene-(5- to 15-membered heterocycloalkyl), wherein each heterocycloalkyl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more —OH, halogen, or C₁-C₄ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 6-membered heterocycloalkyl, C₆-C₁₀ aryl, or 5- to 10-membered heteroaryl; and
R⁸ is independently, at each occurrence, halogen or —C₁-C₄ alkyl.

In some embodiments, R¹ and R² are each independently selected from —H, -5- to 15-membered heterocycloalkyl, and —C₁-C₄ alkylene-(5- to 15-membered heterocycloalkyl), wherein each heterocycloalkyl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more —OH, halogen, or C₁-C₄ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 6-membered heterocycloalkyl, C₆-C₁₀ aryl, or 5- to 10-membered heteroaryl; and
R⁸ is independently, at each occurrence, halogen or —C₁-C₄ alkyl.

In some embodiments, R¹ and R² are each independently selected from —H, -5- to 12-membered heterocycloalkyl, and —C₁-C₄ alkylene-(5- to 12-membered heterocycloalkyl), wherein each heterocycloalkyl is optionally substituted with one or more R³; or
R¹ and R², together with the nitrogen atom to which they are attached, combine to form a 5- to 12-membered heterocycloalkyl, wherein said heterocycloalkyl is optionally substituted with one or more R⁴;
R³ is independently, at each occurrence, —C₁-C₄ alkyl, —C₃-C₁₀ cycloalkyl, —C₁-C₄ alkylene-C₃-C₁₀ cycloalkyl, 5- to 12-membered heterocycloalkyl, —C₁-C₄ alkylene-(5- to 12-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more C₁-C₄ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₁₀ cycloalkyl, —C₁-C₄ alkylene-C₅-C₁₀ cycloalkyl, 5- to 12-membered heterocycloalkyl, —C₁-C₄ alkylene-(5- to 12-membered heterocycloalkyl), phenyl, —C₁-C₄ alkylene-phenyl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ is —H;
R² is independently selected from -5- to 6-membered heterocycloalkyl and —C₁-C₂ alkylene-(5- to 6-membered heterocycloalkyl), wherein each heterocycloalkyl is optionally substituted with one or more R³; or
R¹ and R², together with the nitrogen atom to which they are attached, combine to form a 6- to 7-membered heterocycloalkyl, wherein said heterocycloalkyl is optionally substituted with one or more R⁴;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, —C₁-C₂ alkylene-C₆ aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more C₁-C₂ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₆ cycloalkyl, —C₁-C₄ alkylene-C₅-C₆ cycloalkyl, 5- to 6-membered heterocycloalkyl, —C₁-C₄ alkylene-(5- to 6-membered heterocycloalkyl), phenyl, —C₁-C₄ alkylene-phenyl, 5- to 6-membered heteroaryl, or —C₁-C₄ alkylene-(5- to 6-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is optionally substituted with one or more R³; or
R¹ and R², together with the nitrogen atom to which they are attached, combine to form a 6- to 7-membered heterocycloalkyl, wherein said heterocycloalkyl is optionally substituted with one or more R⁴;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more C₁-C₂ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₆ cycloalkyl, —C₁-C₄ alkylene-C₅-C₆ cycloalkyl, 5- to 6-membered heterocycloalkyl, —C₁-C₄ alkylene-(5- to 6-membered heterocycloalkyl), phenyl, —C₁-C₄ alkylene-phenyl, 5- to 6-membered heteroaryl, or —C₁-C₄ alkylene-(5- to 6-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine, piperidine, morpholine, and piperazine, and wherein each heterocycloalkyl is optionally substituted with one or more R³; or R¹ and R², together with the nitrogen atom to which they are attached, combine to form a 6- to 7-membered heterocycloalkyl, wherein said heterocycloalkyl is optionally substituted with one or more R⁴;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, or —C₁-C₂ alkylene-C₆ aryl, wherein each cycloalkyl, heterocycloalkyl, or aryl is optionally substituted with one or more C₁-C₂ alkyl;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, 5- to 6-membered heterocycloalkyl, —C₁-C₂ alkylene-(5- to 6-membered heterocycloalkyl), phenyl, —C₁-C₂ alkylene-phenyl, 5- to 6-membered heteroaryl, or —C₁-C₂ alkylene-(5- to 6-membered heteroaryl), wherein each alkyl, cycloalkyl, heterocycloalkyl, phenyl or heteroaryl is optionally substituted with one or more R⁸;
R⁶ and R⁷ are each independently, at each occurrence, —H, —C₁-C₂ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or more R³; or
R¹ and R², together with the nitrogen atom to which they are attached, combine to form a 6- to 7-membered heterocycloalkyl, wherein said heterocycloalkyl is selected from piperidine, piperazine, and 1,4-diazepane, and wherein said heterocycloalkyl is optionally substituted with one or more R⁴;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, or —C₁-C₂ alkylene-C₆ aryl; wherein each cycloalkyl, heterocycloalkyl, or aryl is optionally substituted with one or more C₁-C₂ alkyl;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, 5- to 6-membered heterocycloalkyl, —C₁-C₂ alkylene-(5- to 6-membered heterocycloalkyl), phenyl, —C₁-C₂ alkylene-phenyl, 5- to 6-membered heteroaryl, or —C₁-C₂ alkylene-(5- to 6-membered heteroaryl), wherein each heterocycloalkyl is selected from piperidine, piperazine, and morpholine, and wherein each alkyl, cycloalkyl, heterocycloalkyl, phenyl or heteroaryl is optionally substituted with one or more R⁸;
R⁶ and R⁷ are each independently, at each occurrence, —H, —C₁-C₂ alkyl, or phenyl; and
R⁸ is independently, at each occurrence, halogen or —C₁-C₄ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or two R³; or
R¹ and R², together with the nitrogen atom to which they are attached, combine to form a 6- to 7-membered heterocycloalkyl, wherein said heterocycloalkyl is selected from piperidine and piperazine, and wherein said heterocycloalkyl is optionally substituted with one R⁴;
R³ is independently, at each occurrence, —C₁-C₄ alkyl, —C₁-C₂ alkylene-C₅-C₆ cycloalkyl, 5- to 6-membered heterocycloalkyl, or —C₁-C₂ alkylene-C₆ aryl; wherein each heterocycloalkyl is optionally substituted with one or more C₁-C₂ alkyl;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, 5- to 6-membered heterocycloalkyl, —C₁-C₂ alkylene-(5- to 6-membered heterocycloalkyl), phenyl, —C₁-C₂ alkylene-phenyl, 5- to 6-membered heteroaryl, or —C₁-C₂ alkylene-(5- to 6-membered heteroaryl), wherein each heterocycloalkyl is selected from piperidine, piperazine, and morpholine, preferably piperidine and morpholine, and wherein each alkyl, cycloalkyl, heterocycloalkyl, phenyl or heteroaryl is unsubstituted;
R⁶ and R⁷ are each independently, at each occurrence, —H, —C₁-C₂ alkyl, or phenyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or two R³; or
R¹ and R², together with the nitrogen atom to which they are attached, combine to form a 6- to 7-membered heterocycloalkyl, wherein said heterocycloalkyl is selected from piperidine and piperazine, and wherein said heterocycloalkyl is optionally substituted at the 4-position with one R⁴;
R³ is independently, at each occurrence, —C₁-C₂ alkylene-C₆ aryl;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, phenyl, —C₁-C₂ alkylene-phenyl, 5- to 6-membered heteroaryl, or —C₁-C₂ alkylene-(5- to 6-membered heteroaryl), wherein each alkyl, cycloalkyl, phenyl or heteroaryl is unsubstituted; and
R⁶ and R⁷ are each independently, at each occurrence, —H, —C₁-C₂ alkyl, or phenyl.

In some embodiments, R¹ and R² are each independently selected from —H, C₁-C₄ alkyl, C₃-C₆ cycloalkyl, 5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each alkyl, cycloalkyl and heterocycloalkyl is optionally substituted by one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —CN, —OC₁-C₂ alkyl, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆ aryl, —C₁-C₂ alkylene-C₆ aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more —C₁-C₂ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In some embodiments, R¹ and R² are independently selected from —H, C₁-C₄ alkyl, C₃-C₆ cycloalkyl, -5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, or —C₁-C₂ alkylene-C₆ aryl; wherein each cycloalkyl, heterocycloalkyl, or aryl is optionally substituted with one or more C₁-C₂ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In some embodiments, R¹ and R² are each independently selected from —H, —C₁-C₆ alkyl, —C₃-C₆ cycloalkyl, C₁-C₆ alkylene-C₃-C₆ cycloalkyl, -5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, or —C₁-C₆ alkylene-(5- to 6-membered heteroaryl), wherein each cycloalkyl, heterocycloalkyl, aryl, or heteroaryl is optionally substituted with one or more R³.

In some embodiments, R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³;
R³ is independently, at each occurrence, —OH, halogen, —CN, —OC₁-C₆ alkyl, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl); wherein each cycloalkyl, heterocycloalkyl, aryl, or heteroaryl is optionally substituted with one or more —OH, halogen, or C₁-C₆ alkyl.

In some embodiments, R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³;
R³ is independently, at each occurrence, —OH, halogen, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl); wherein each cycloalkyl, heterocycloalkyl, aryl, or heteroaryl is optionally substituted with one or more —OH, halogen, or C₁-C₆ alkyl.

In some embodiments, R¹ and R² are each independently selected from —H, —C₁-C₄ alkyl, —C₃-C₁₀ cycloalkyl, C₁-C₄ alkylene-C₃-C₁₀ cycloalkyl, -5- to 15-membered heterocycloalkyl, and —C₁-C₄ alkylene-(5- to 15-membered heterocycloalkyl), wherein each alkyl, cycloalkyl, or heterocycloalkyl is optionally substituted with one or more R³;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄alkylene-(5- to 10-membered heteroaryl); wherein each cycloalkyl, heterocycloalkyl, aryl, or heteroaryl is optionally substituted with one or more —OH, halogen, or C₁-C₄ alkyl.

In some embodiments, R¹ and R² are each independently selected from —H, C₁-C₄ alkyl, C₃-C₆ cycloalkyl, -5- to 15-membered heterocycloalkyl, and —C₁-C₄ alkylene-(5- to 15-membered heterocycloalkyl), wherein each heterocycloalkyl is optionally substituted with one or more R³;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more —OH, halogen, or C₁-C₄ alkyl.

In some embodiments, R¹ and R² are each independently selected from —H, -5- to 15-membered heterocycloalkyl, and —C₁-C₄ alkylene-(5- to 15-membered heterocycloalkyl), wherein each heterocycloalkyl is optionally substituted with one or more R³;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more —OH, halogen, or C₁-C₄ alkyl.

In some embodiments, R¹ and R² are each independently selected from —H, -5- to 12-membered heterocycloalkyl, and —C₁-C₄ alkylene-(5- to 12-membered heterocycloalkyl), wherein each heterocycloalkyl is optionally substituted with one or more R³;
R³ is independently, at each occurrence, —C₁-C₄ alkyl, —C₃-C₁₀ cycloalkyl, —C₁-C₄ alkylene-C₃-C₁₀ cycloalkyl, 5- to 12-membered heterocycloalkyl, —C₁-C₄ alkylene-(5- to 12-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more C₁-C₄ alkyl.

In some embodiments, R¹ and R² are each independently selected from —H, C₁-C₄ alkyl, C₃-C₆ cycloalkyl, 5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each alkyl, cycloalkyl and heterocycloalkyl is optionally substituted by one or more R³;
R³ is independently, at each occurrence, —CN, —OC₁-C₂ alkyl, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆ aryl, —C₁-C₂ alkylene-C₆ aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more —C₁-C₂ alkyl.

In some embodiments, R¹ and R² are independently selected from —H, C₁-C₄ alkyl, C₃-C₆ cycloalkyl, -5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or more R³;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, or —C₁-C₂ alkylene-C₆ aryl; wherein each cycloalkyl, heterocycloalkyl, or aryl is optionally substituted with one or more C₁-C₂ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is optionally substituted with one or more R³;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, —C₁-C₂ alkylene-C₆ aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more C₁-C₂ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is optionally substituted with one or more R³;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more C₁-C₂ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine, piperidine, morpholine, and piperazine, and wherein each heterocycloalkyl is optionally substituted with one or more R³;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, or —C₁-C₂ alkylene-C₆ aryl, wherein each cycloalkyl, heterocycloalkyl, or aryl is optionally substituted with one or more C₁-C₂ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or more R³;
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, or —C₁-C₂ alkylene-C₆ aryl; wherein each cycloalkyl, heterocycloalkyl, or aryl is optionally substituted with one or more C₁-C₂ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or two R³;
R³ is independently, at each occurrence, —C₁-C₄ alkyl, —C₁-C₂ alkylene-C₅-C₆ cycloalkyl, 5- to 6-membered heterocycloalkyl, or —C₁-C₂ alkylene-C₆ aryl; wherein each heterocycloalkyl is optionally substituted with one or more C₁-C₂ alkyl.

In some embodiments, R¹ is —H;
R² is independently selected from −5- to 6-membered heterocycloalkyl and —C₁-C₂ alkylene-(5- to 6-membered heterocycloalkyl), wherein each heterocycloalkyl is selected from pyrrolidine and piperidine, preferably piperidine, and wherein each heterocycloalkyl is optionally substituted with one or two R³;
R³ is independently, at each occurrence, —C₁-C₂ alkylene-C₆ aryl.

In some embodiments, R¹ and R², together with the nitrogen atom to which they are attached, combine to form a 5- to 15-membered heterocycloalkyl, wherein said heterocycloalkyl is optionally substituted with one or more R⁴.

In some embodiments, R¹ and R², together with the nitrogen atom to which they are attached, combine to form a 5- to 15-membered heterocycloalkyl, wherein said heterocycloalkyl is optionally substituted with one or more R⁴;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

In some embodiments, R¹ and R², together with the nitrogen atom to which they are attached, combine to form a 5- to 15-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 10-membered heterocycloalkyl, C₆-C₁₀ aryl, or 5- to 10-membered heteroaryl; and
R⁸ is independently, at each occurrence, —OH, halogen, or —C₁-C₄ alkyl.

In some embodiments, R¹ and R², together with the nitrogen atom to which they are attached, combine to form a 5- to 15-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 6-membered heterocycloalkyl, C₆-C₁₀ aryl, or 5- to 10-membered heteroaryl; and R⁸ is independently, at each occurrence, halogen or —C₁-C₄ alkyl.

In some embodiments, R¹ and R², together with the nitrogen atom to which they are attached, combine to form a 5- to 12-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₁₀ cycloalkyl, —C₁-C₄ alkylene-C₅-C₁₀ cycloalkyl, 5- to 12-membered heterocycloalkyl, —C₁-C₄ alkylene-(5- to 12-membered heterocycloalkyl), phenyl, —C₁-C₄ alkylene-phenyl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ and R², together with the nitrogen atom to which they are attached, combine to form a 5- to 12-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₆ cycloalkyl, —C₁-C₄ alkylene-C₅-C₆ cycloalkyl, 5- to 6-membered heterocycloalkyl, —C₁-C₄ alkylene-(5- to 6-membered heterocycloalkyl), phenyl, —C₁-C₄ alkylene-phenyl, 5- to 6-membered heteroaryl, or —C₁-C₄ alkylene-(5- to 6-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ and R², together with the nitrogen atom to which they are attached, combine to form a 6- to 7-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₆ cycloalkyl, —C₁-C₄ alkylene-C₅-C₆ cycloalkyl, 5- to 6-membered heterocycloalkyl, —C₁-C₄ alkylene-(5- to 6-membered heterocycloalkyl), phenyl, —C₁-C₄ alkylene-phenyl, 5- to 6-membered heteroaryl, or —C₁-C₄ alkylene-(5- to 6-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ and R², together with the nitrogen atom to which they are attached, combine to form a 6- to 7-membered heterocycloalkyl, wherein said heterocycloalkyl is optionally substituted with one or more R⁴;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, 5- to 6-membered heterocycloalkyl, —C₁-C₂ alkylene-(5- to 6-membered heterocycloalkyl), phenyl, —C₁-C₂ alkylene-phenyl, 5- to 6-membered heteroaryl, or —C₁-C₂ alkylene-(5- to 6-membered heteroaryl), wherein each alkyl, cycloalkyl, heterocycloalkyl, phenyl or heteroaryl is optionally substituted with one or more R⁸;
R⁶ and R⁷ are each independently, at each occurrence, —H, —C₁-C₂ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

In some embodiments, R¹ and R², together with the nitrogen atom to which they are attached, combine to form a 6- to 7-membered heterocycloalkyl, wherein said heterocycloalkyl is selected from piperidine, piperazine, and 1,4-diazepane, and wherein said heterocycloalkyl is optionally substituted with one or more R⁴;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, 5- to 6-membered heterocycloalkyl, —C₁-C₂ alkylene-(5- to 6-membered heterocycloalkyl), phenyl, —C₁-C₂ alkylene-phenyl, 5- to 6-membered heteroaryl, or —C₁-C₂ alkylene-(5- to 6-membered heteroaryl), wherein each heterocycloalkyl is selected from piperidine, piperazine, and morpholine, and wherein each alkyl, cycloalkyl, heterocycloalkyl, phenyl or heteroaryl is optionally substituted with one or more R⁸;
R⁶ and R⁷ are each independently, at each occurrence, —H, —C₁-C₂ alkyl, or phenyl; and
R⁸ is independently, at each occurrence, halogen or —C₁-C₄ alkyl.

In some embodiments, R¹ and R², together with the nitrogen atom to which they are attached, combine to form a 6- to 7-membered heterocycloalkyl, wherein said heterocycloalkyl is selected from piperidine and piperazine, and wherein said heterocycloalkyl is optionally substituted with one R⁴;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, 5- to 6-membered heterocycloalkyl, —C₁-C₂ alkylene-(5- to 6-membered heterocycloalkyl), phenyl, —C₁-C₂ alkylene-phenyl, 5- to 6-membered heteroaryl, or —C₁-C₂ alkylene-(5- to 6-membered heteroaryl), wherein each heterocycloalkyl is selected from piperidine, piperazine, and morpholine, preferably piperidine and morpholine, and wherein each alkyl, cycloalkyl, heterocycloalkyl, phenyl or heteroaryl is unsubstituted;
R⁶ and R⁷ are each independently, at each occurrence, —H, —C₁-C₂ alkyl, or phenyl.

In some embodiments, R¹ and R², together with the nitrogen atom to which they are attached, combine to form a 6- to 7-membered heterocycloalkyl, wherein said heterocycloalkyl is selected from piperidine and piperazine, and wherein said heterocycloalkyl is optionally substituted at the 4-position with one R⁴;
R⁴ is independently, at each occurrence, —C₁-C₆ alkyl, —NR⁶R⁷, —C₁-C₂ alkylene-NR⁶R⁷, —C₅-C₆ cycloalkyl, —C₁-C₂ alkylene-C₅-C₆ cycloalkyl, phenyl, —C₁-C₂ alkylene-phenyl, 5- to 6-membered heteroaryl, or —C₁-C₂ alkylene-(5- to 6-membered heteroaryl), wherein each alkyl, cycloalkyl, phenyl or heteroaryl is unsubstituted; and
R⁶ and R⁷ are each independently, at each occurrence, —H, —C₁-C₂ alkyl, or phenyl.

In one or more embodiments, R¹ and R² of a compound of the present invention can form one of the structures selected from Table 1, below:

TABLE 1
Embodiments of R¹ and R²

| Embodiment | Structure |
|---|---|
| 1 | 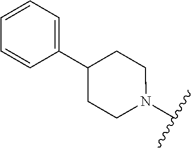 [SMILES: *N1CCC(c2ccccc2)CC1] |
| 2 | 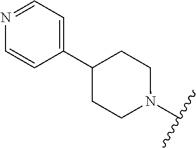 [SMILES: *N1CCC(c2ccncc2)CC1] |
| 3 | 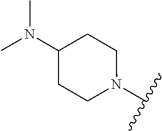 [SMILES: *N1CCC(N(C)C)CC1] |
| 4 | 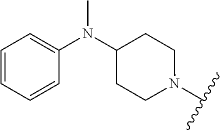 [SMILES: *N1CCC(N(C)c2ccccc2)CC1] |
| 5 | 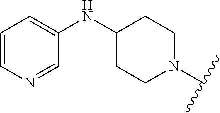 [SMILES: *N1CCC(Nc2cccnc2)CC1] |
| 6 | 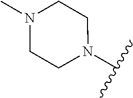 [SMILES: *N1CCN(C)CC1] |
| 7 | 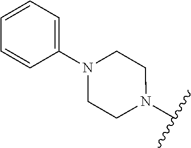 [SMILES: *N1CCN(c2ccccc2)CC1] |
| 8 | 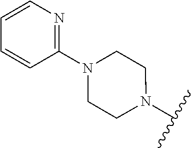 [SMILES: *N1CCN(c2ccccn2)CC1] |
| 9 | 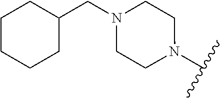 [SMILES: *N1CCN(CC2CCCCC2)CC1] |
| 10 | 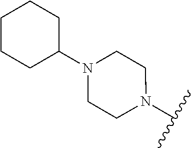 [SMILES: *N1CCN(C2CCCCC2)CC1] |
| 11 | 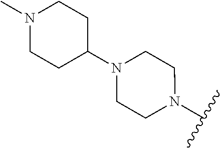 [SMILES: *N1CCN(C2CCN(C)CC2)CC1] |
| 12 | 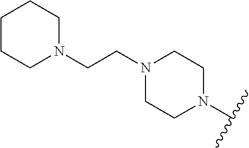 [SMILES: *N1CCN(CCN2CCCCC2)CC1] |
| 13 | 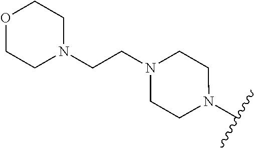 [SMILES: *N1CCN(CCN2CCOCC2)CC1] |
| 14 | 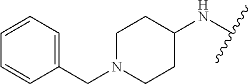 [SMILES: *NC1CCN(Cc2ccccc2)CC1] |
| 15 | 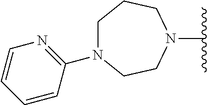 [SMILES: *N1CCCN(c2ccccn2)CC1] |
| 16 | 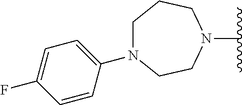 [SMILES: *N1CCCN(c2ccc(F)cc2)CC1] |
| 17 | 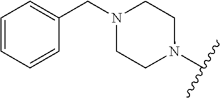 [SMILES: *N1CCN(Cc2ccccc2)CC1] |
| 18 | 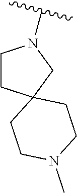 [SMILES: *N1CCC2(CCN(C)CC2)C1] |
| 19 | 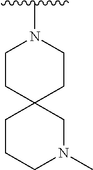 [SMILES: *N1CCC2(CCCN(C)C2)CC1] |
| 20 | 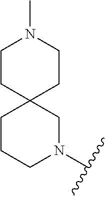 [SMILES: *N1CCCC2(CCN(C)CC2)C1] |
| 21 | 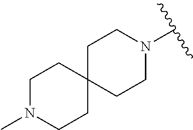 [SMILES: *N1CCC2(CC1)CCN(C)CC2] |
| 22 | 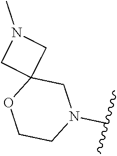 [SMILES: *N1CCOC2(C1)CN(C)C2] |
| 23 | 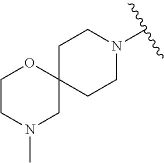 [SMILES: *N1CCC2(CC1)CN(C)CCO2] |
| 24 | 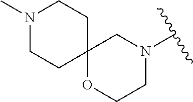 [SMILES: *N1CCOC2(CCN(C)CC2)C1] |
| 25 | 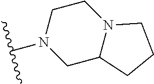 [SMILES: *N1CCN2CCCC2C1] |
| 26 | 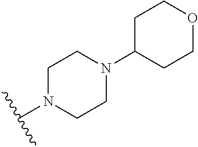 [SMILES: *N1CCN(C2CCOCC2)CC1] |
| 27 | 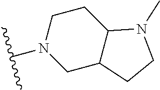 [SMILES: *N1CCC2C(CCN2C)C1] |
| 28 | 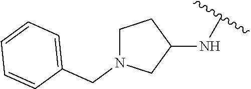 [SMILES: *NC1CCN(Cc2ccccc2)C1] |
| 29 | 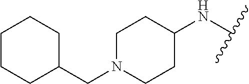 [SMILES: *NC1CCN(CC2CCCCC2)CC1] |
| 30 | 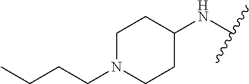 [SMILES: *NC1CCN(CCCC)CC1] |
| 31 | 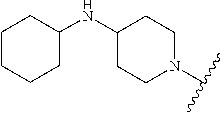 [SMILES: *N1CCC(NC2CCCCC2)CC1] |
| 32, preferably 32a | 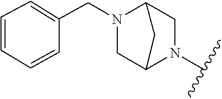 [SMILES: *N1CC2CC1CN2Cc1ccccc1] |
| | preferably |
| | 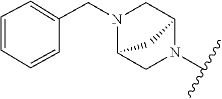 [SMILES: *N1C[C@@H]2C[C@H]1CN2Cc1ccccc1] |
| 33 | 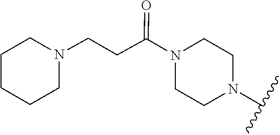 [SMILES: *N1CCN(C(=O)CCN2CCCCC2)CC1] |
| 34 | 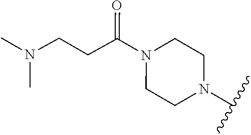 [SMILES: *N1CCN(C(=O)CCN(C)C)CC1] |
| 35 | 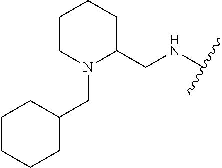 [SMILES: *NCC1CCCCN1CC1CCCCC1] |
| 36 | 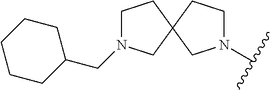 [SMILES: *N1CCC2(CCN(CC3CCCCC3)C2)C1] |
| 37 | 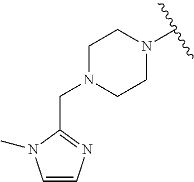 [SMILES: *N1CCN(Cc2nccn2C)CC1] |
| 38 | 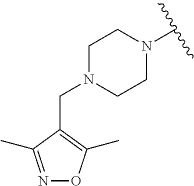 [SMILES: *N1CCN(Cc2c(C)noc2C)CC1] |
| 39 | 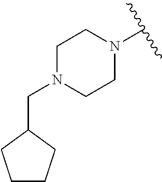 [SMILES: *N1CCN(CC2CCCC2)CC1] |
| 40 | 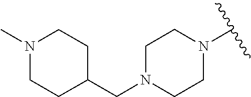 [SMILES: *N1CCN(CC2CCN(C)CC2)CC1] |
| 41 | 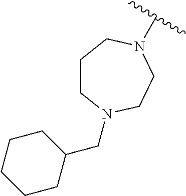 [SMILES: *N1CCCN(CC2CCCCC2)CC1] |
| 42 | 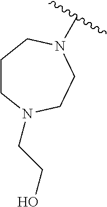 [SMILES: *N1CCCN(CCO)CC1] |
| 43 | 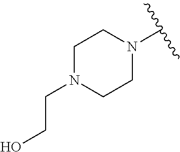 [SMILES: *N1CCN(CCO)CC1] |
| 44 | 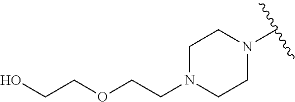 [SMILES: *N1CCN(CCOCCO)CC1] |
| 45 | 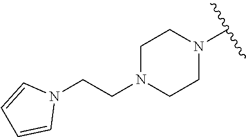 [SMILES: *N1CCN(CCn2cccc2)CC1] |
| 46 | 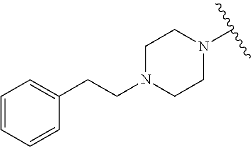 [SMILES: *N1CCN(CCc2ccccc2)CC1] |
| 47 | 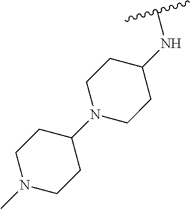 [SMILES: *NC1CCN(C2CCN(C)CC2)CC1] |
| 48 | 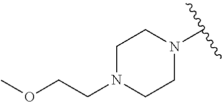 [SMILES: *N1CCN(CCOC)CC1] |
| 49 | 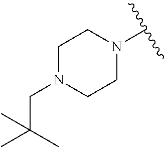 [SMILES: *N1CCN(CC(C)(C)C)CC1] |
| 50 | 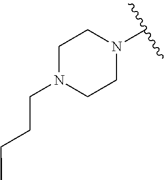 [SMILES: *N1CCN(CCCC)CC1] |
| 51 | 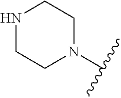 [SMILES: *N1CCNCC1] |
| 52 | 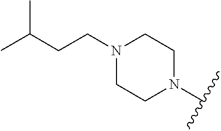 [SMILES: *N1CCN(CCC(C)C)CC1] |
| 53 | 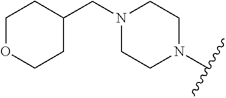 [SMILES: *N1CCN(CC2CCOCC2)CC1] |
| 54 | 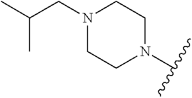 [SMILES: *N1CCN(CC(C)C)CC1] |
| 55 | 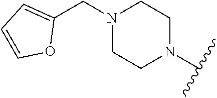 [SMILES: *N1CCN(Cc2ccco2)CC1] |
| 56 | 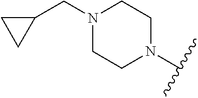 [SMILES: *N1CCN(CC2CC2)CC1] |
| 57 | 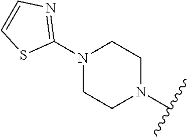 [SMILES: *N1CCN(c2nccs2)CC1] |
| 58 | 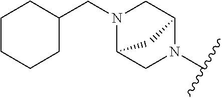 [SMILES: *N1C[C@@H]2C[C@H]1CN2CC1CCCCC1] |
| 59 | 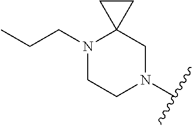 [SMILES: *N1CCN(CCC)C2(CC2)C1] |
| 60 | 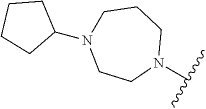 [SMILES: *N1CCCN(C2CCCC2)CC1] |
| 61 | 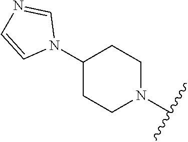 [SMILES: *N1CCC(n2ccnc2)CC1] |
| 62 | 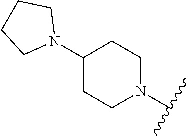 [SMILES: *N1CCC(N2CCCC2)CC1] |
| 63 | 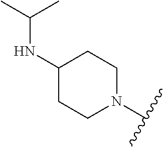 [SMILES: *N1CCC(NC(C)C)CC1] |
| 64 | 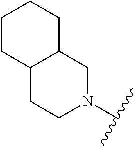 [SMILES: *N1CCC2CCCCC2C1] |
| 65 | 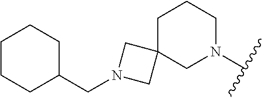 [SMILES: *N1CCCC2(C1)CN(CC1CCCCC1)C2] |
| 66 | 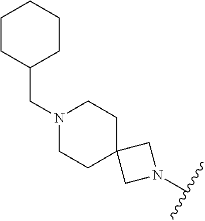 [SMILES: *N1CC2(CCN(CC3CCCCC3)CC2)C1] |
| 67 | 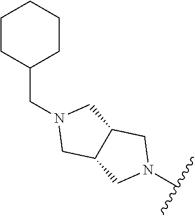 [SMILES: *N1C[C@@H]2CN(CC3CCCCC3)C[C@@H]2C1] |
| 68 | 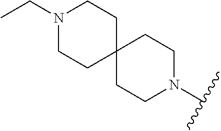 [SMILES: *N1CCC2(CC1)CCN(CC)CC2] |
| 69 | 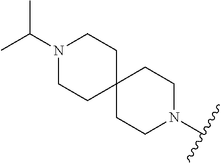 [SMILES: *N1CCC2(CC1)CCN(C(C)C)CC2] |
| 70 | 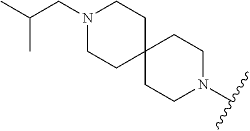 [SMILES: *N1CCC2(CC1)CCN(CC(C)C)CC2] |
| 71 | 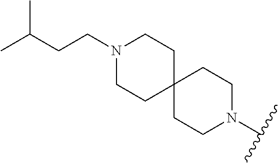 [SMILES: *N1CCC2(CC1)CCN(CCC(C)C)CC2] |
| 72 | 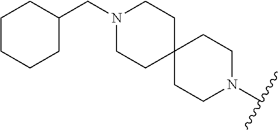 [SMILES: *N1CCC2(CC1)CCN(CC1CCCCC1)CC2] |
| 73 | 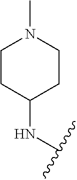 [SMILES: *NC1CCN(C)CC1] |
| 74 | 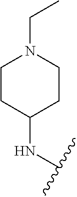 [SMILES: *NC1CCN(CC)CC1] |
| 75 | 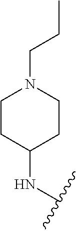 [SMILES: *NC1CCN(CCC)CC1] |
| 76 | 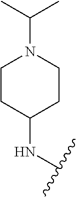 [SMILES: *NC1CCN(C(C)C)CC1] |
| 77 | 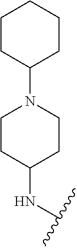 [SMILES: *NC1CCN(C2CCCCC2)CC1] |
| 78 | 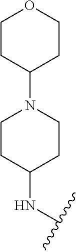 [SMILES: *NC1CCN(C2CCOCC2)CC1] |
| 79 | 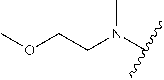 [SMILES: *N(C)CCOC] |
| 80 | 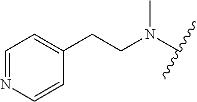 [SMILES: *N(C)CCc1ccncc1] |
| 81 | 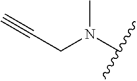 [SMILES: *N(C)CC#C] |
| 82 | 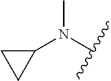 [SMILES: *N(C)C1CC1] |
| 83 | 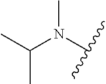 [SMILES: *N(C)C(C)C] |
| 84 | 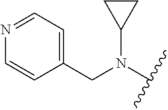 [SMILES: *N(Cc1ccncc1)C1CC1] |

In some embodiments, the compound of Formula I is selected from the group consisting of the compounds shown in Table 2. In one or more embodiments of any of the above aspects, the compound of Formula I is selected from Table 2, below:

TABLE 2
Compound of the Invention

| Cpd | Structure |
|---|---|
| 1 | 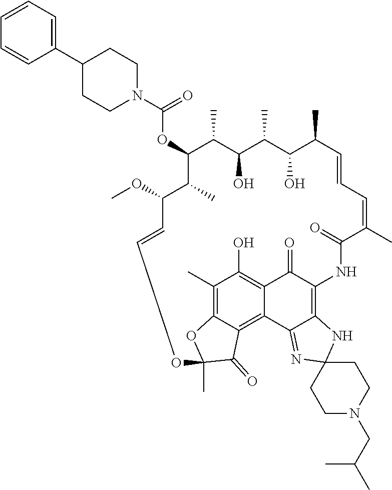 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccccc3)CC2)[C@@H]1C)C4=O] |
| 2 | 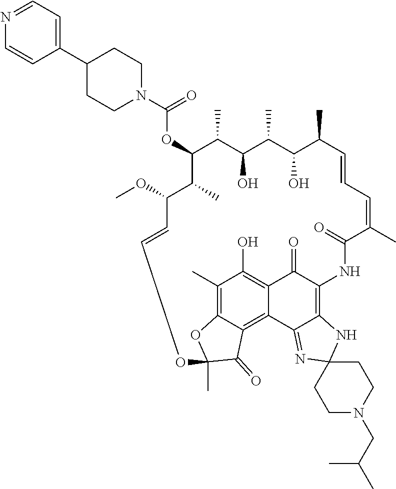 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccncc3)CC2)[C@@H]1C)C4=O] |
| 3 | 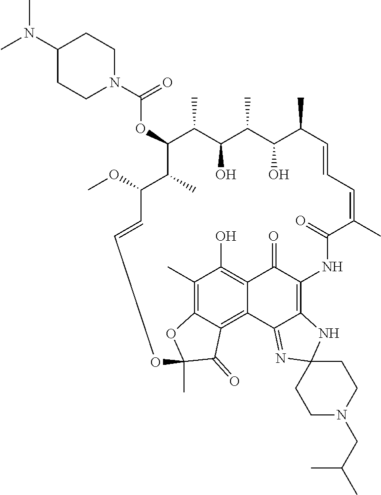 |
| 4 | 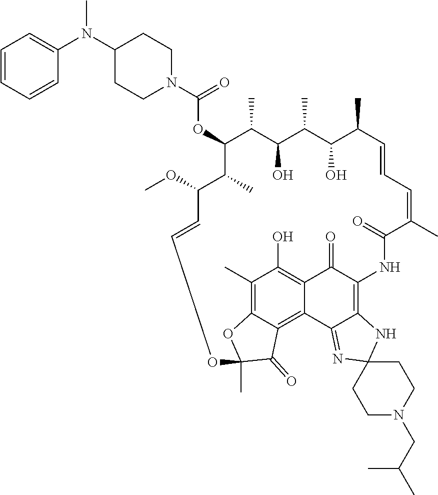 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(N(C)c3ccccc3)CC2)[C@@H]1C)C4=O] |
| 5 | 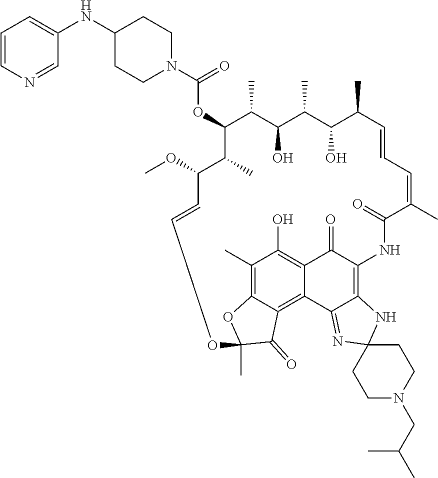 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(Nc3cccnc3)CC2)[C@@H]1C)C4=O] |
| 6 | 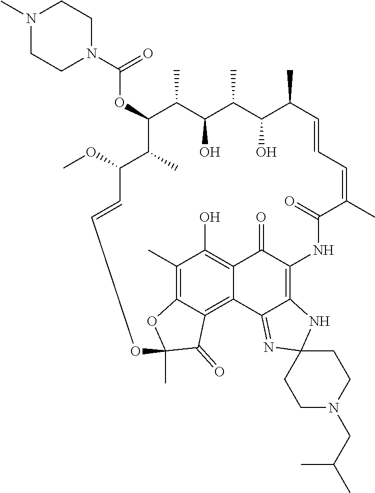 |
| 7 | 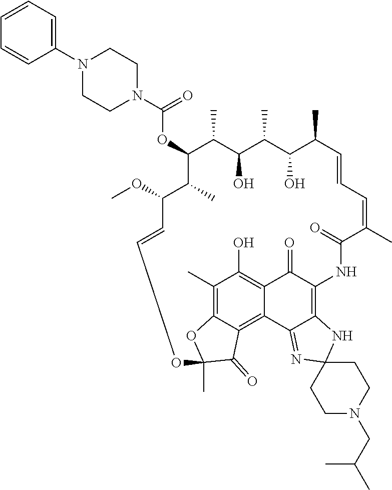 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccc3)CC2)[C@@H]1C)C4=O] |
| 8 | 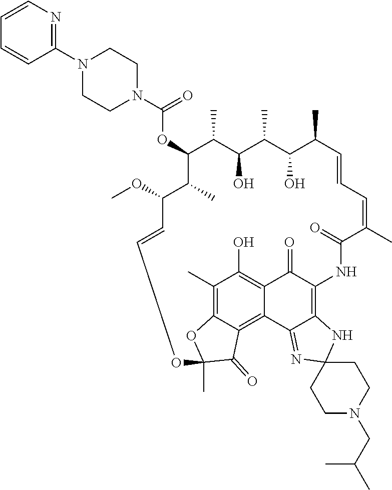 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccn3)CC2)[C@@H]1C)C4=O] |
| 9 | 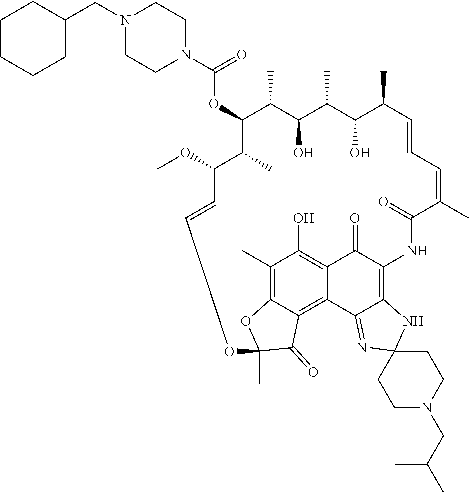 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCCC3)CC2)[C@@H]1C)C4=O] |
| 10 | 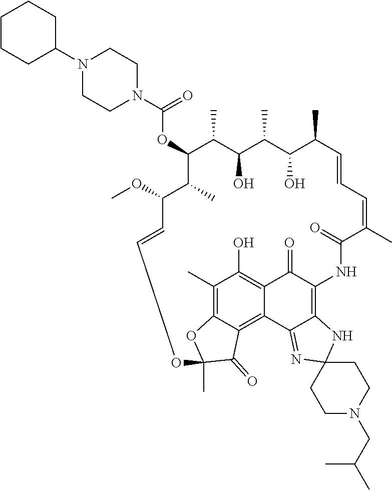 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCCCC3)CC2)[C@@H]1C)C4=O] |
| 11 | 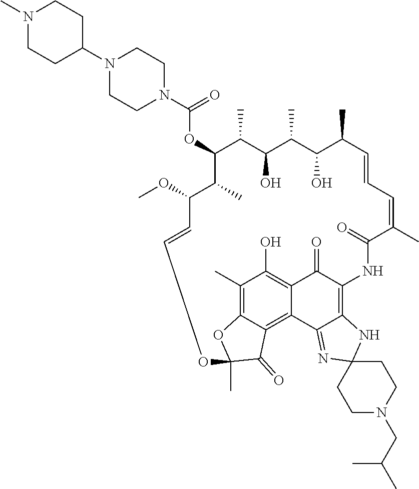 |
| 12 | 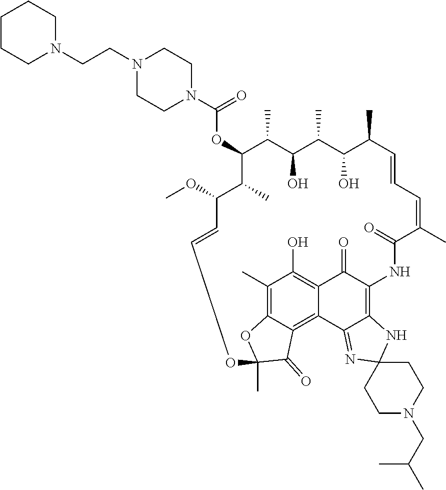 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCCCC3)CC2)[C@@H]1C)C4=O] |
| 13 | 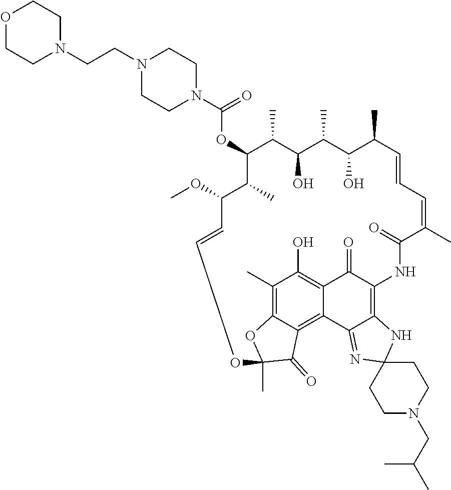 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCOCC3)CC2)[C@@H]1C)C4=O] |
| 14 | 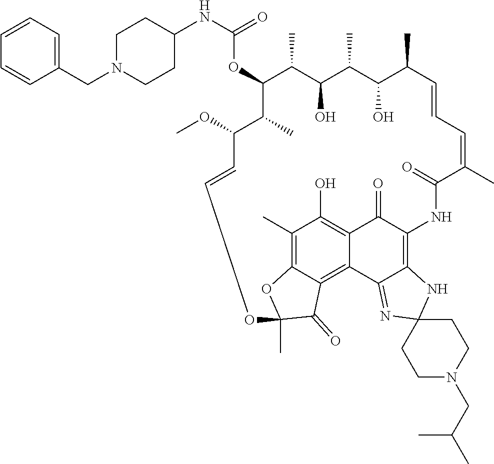 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)CC2)[C@@H]1C)C4=O] |
| 15 | 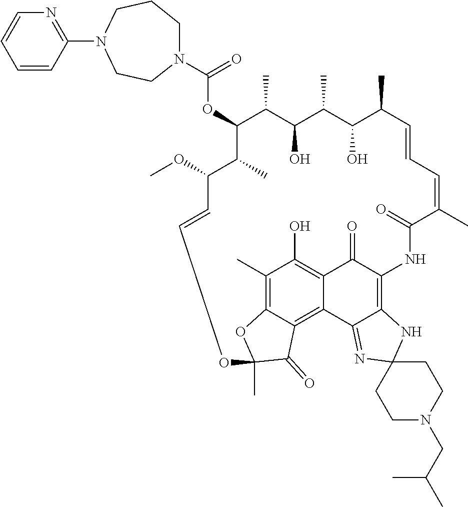 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccccn3)CC2)[C@@H]1C)C4=O] |
| 16 | 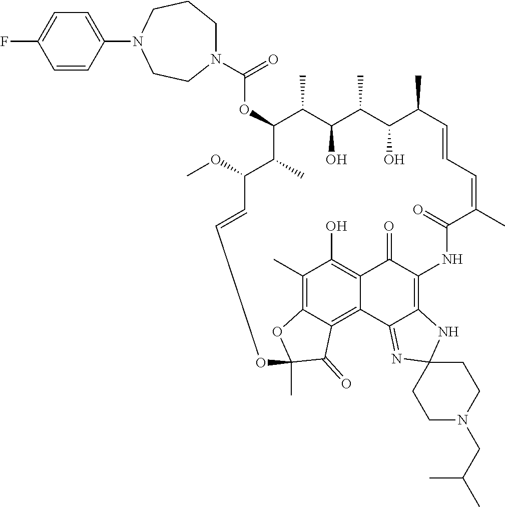 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccc(F)cc3)CC2)[C@@H]1C)C4=O] |
| 17 | 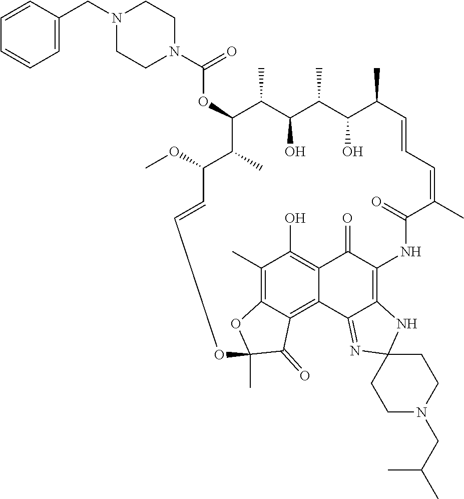 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccccc3)CC2)[C@@H]1C)C4=O] |
| 18 | 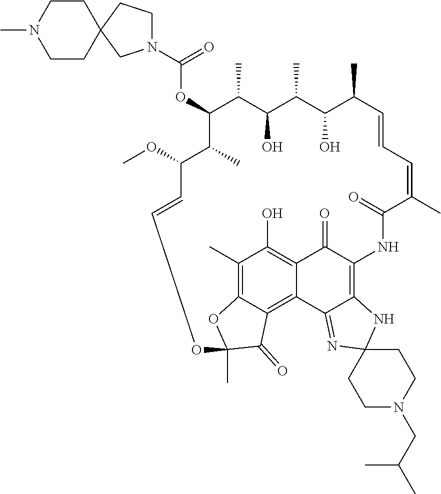 |
| 19 | 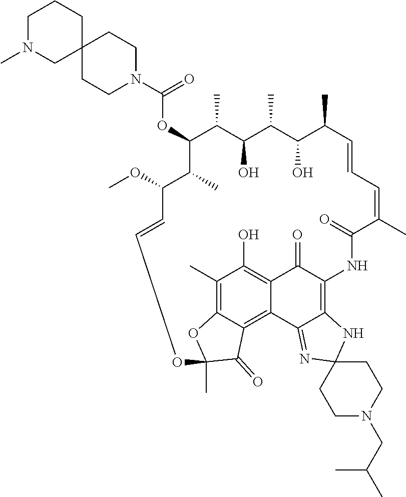 |
| 20 | 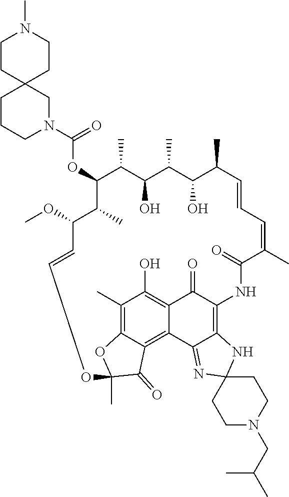 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CCN(C)CC3)C2)[C@@H]1C)C4=O] |
| 21 | 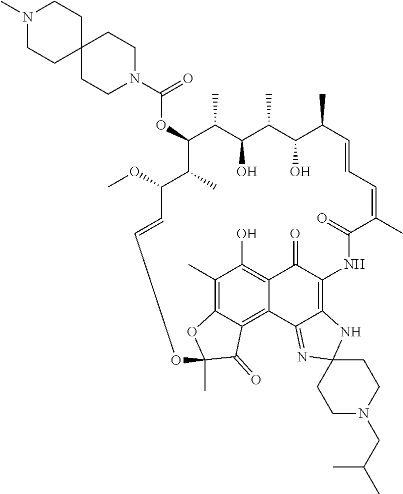 |
| 22 | 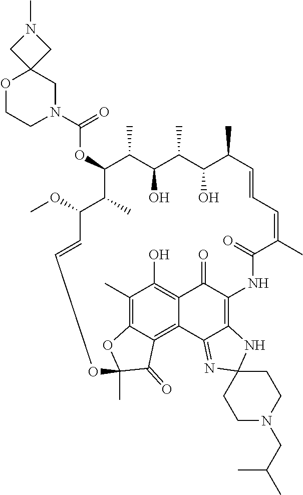 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCOC3(CN(C)C3)C2)[C@@H]1C)C4=O] |
| 23 | 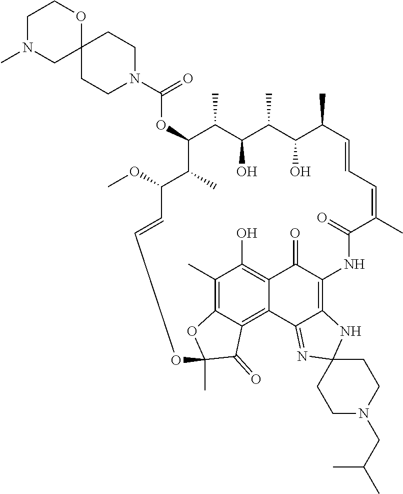 |
| 24 | 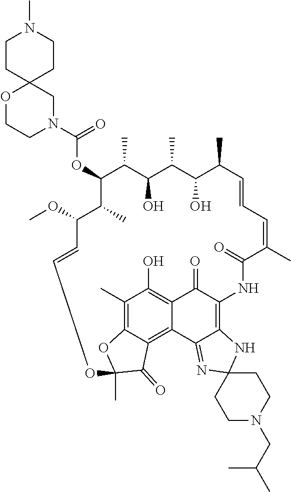 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCOC3(CCN(C)CC3)C2)[C@@H]1C)C4=O] |
| 25 | 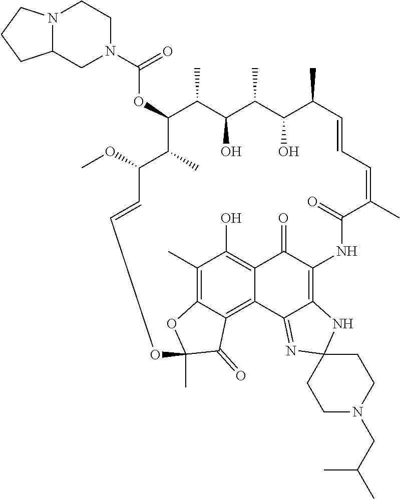 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN3CCCC3C2)[C@@H]1C)C4=O] |
| 26 | 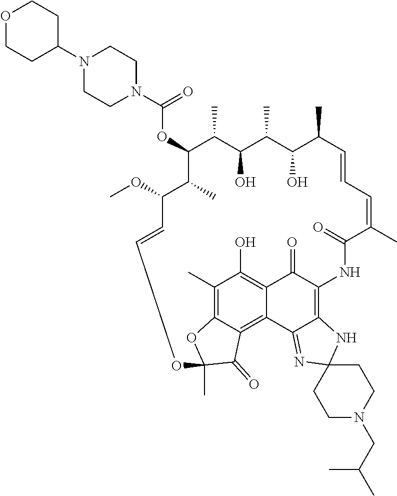 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCOCC3)CC2)[C@@H]1C)C4=O] |
| 27 | 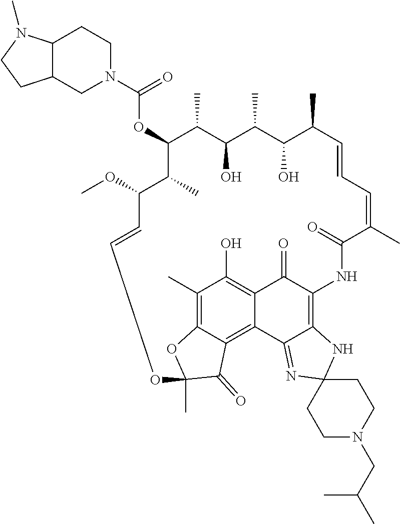 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3C(CCN3C)C2)[C@@H]1C)C4=O] |
| 28 | 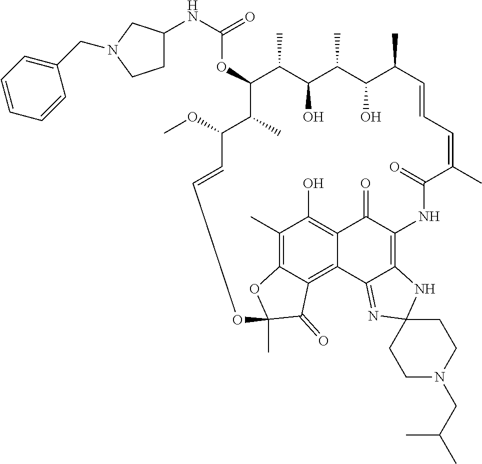 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)C2)[C@@H]1C)C4=O] |
| 29 | 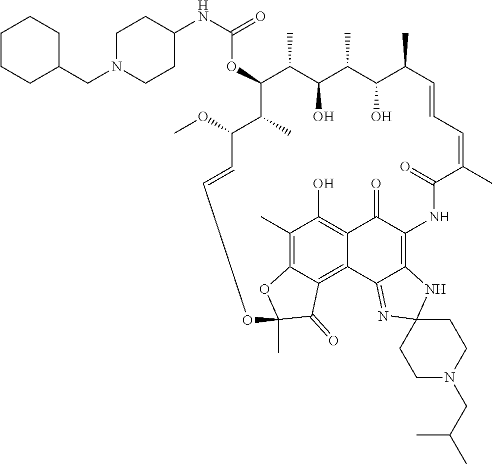 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(CC3CCCCC3)CC2)[C@@H]1C)C4=O] |
| 30 | 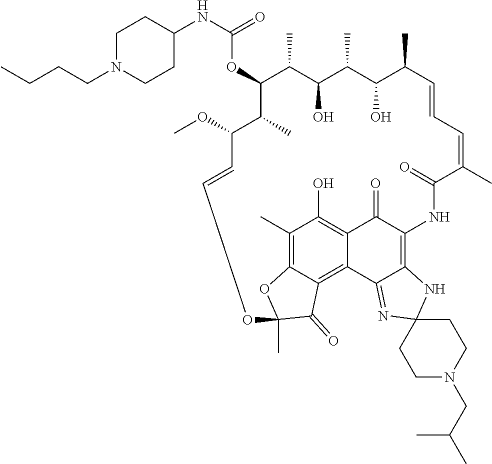 [SMILES: CCCCN1CCC(NC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1] |
| 31 | 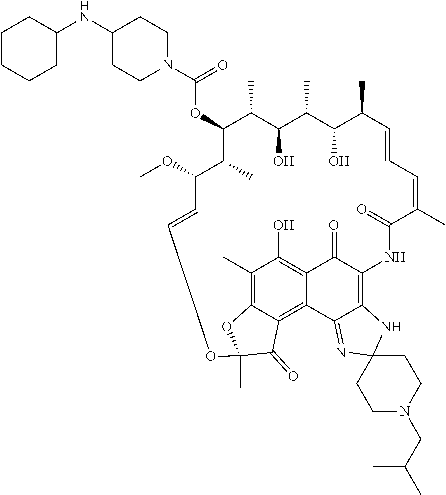 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(NC3CCCCC3)CC2)[C@@H]1C)C4=O] |
| 32, 32a | 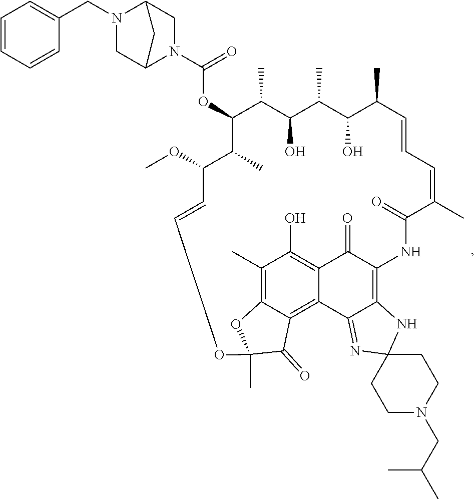 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3CC2CN3Cc2ccccc2)[C@@H]1C)C4=O] |
| | preferably |
| | 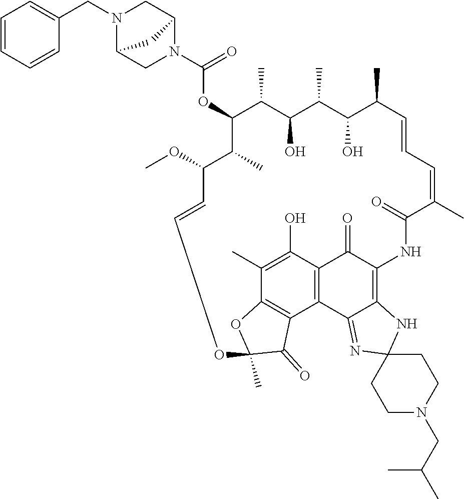 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@@H]3C[C@H]2CN3Cc2ccccc2)[C@@H]1C)C4=O] |
| 33 | 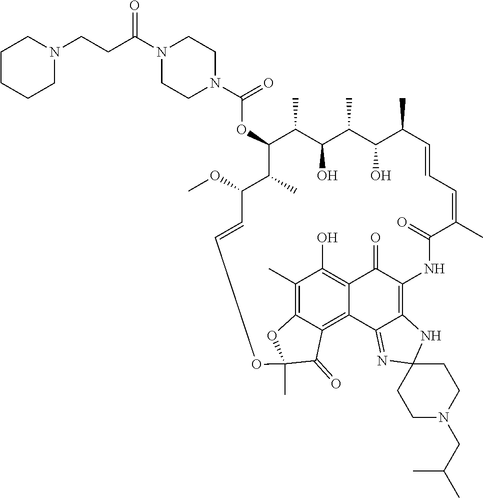 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C(=O)CCN3CCCCC3)CC2)[C@@H]1C)C4=O] |
| 34 | 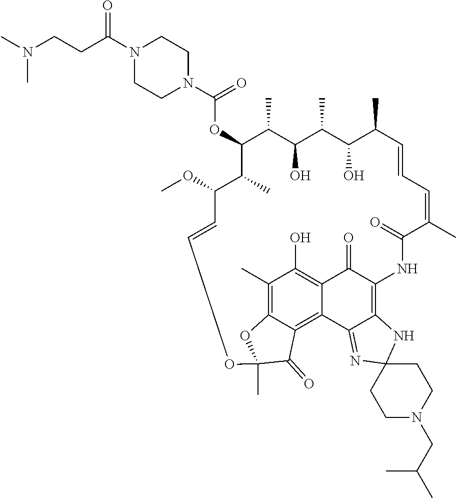 |
| 35 | 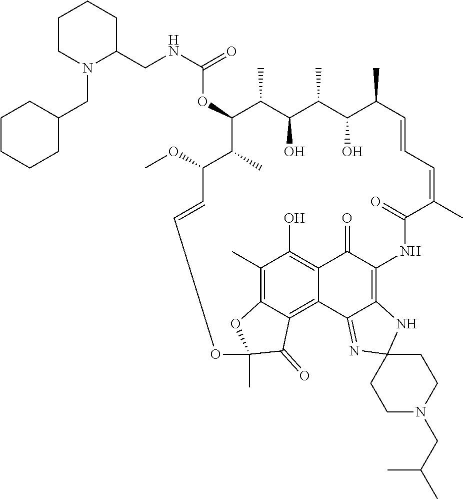 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NCC2CCCCN2CC2CCCCC2)[C@@H]1C)C4=O] |
| 36 | 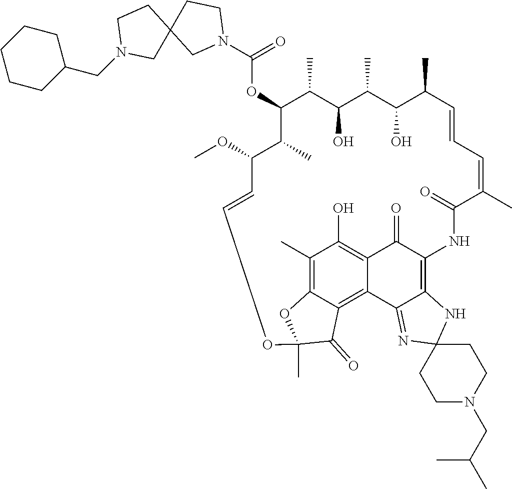 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC5CCCCC5)C3)C2)[C@@H]1C)C4=O] |
| 37 | 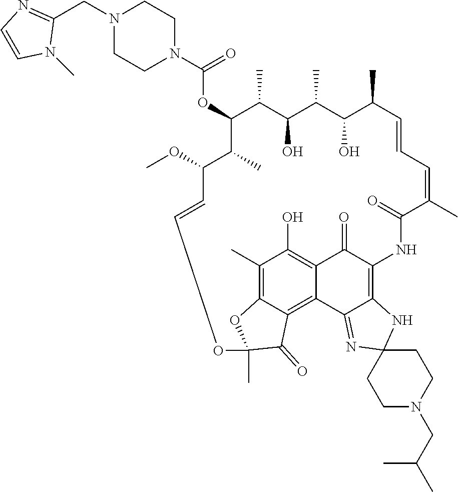 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3nccn3C)CC2)[C@@H]1C)C4=O] |
| 38 | 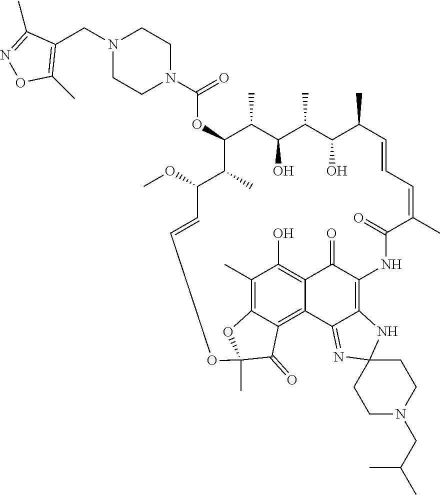 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3c(C)noc3C)CC2)[C@@H]1C)C4=O] |
| 39 | 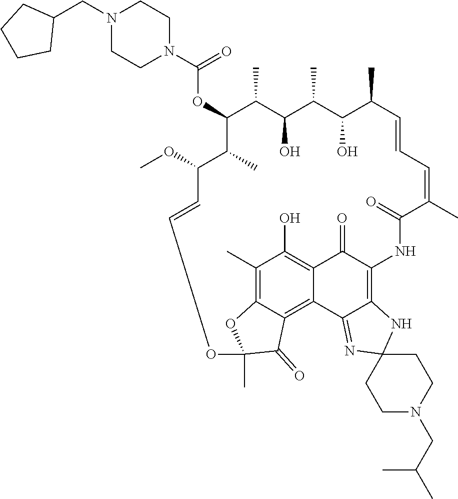 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCC3)CC2)[C@@H]1C)C4=O] |
| 40 | 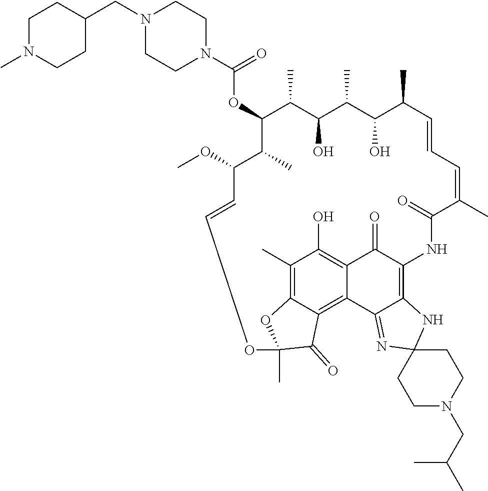 |
| 41 | 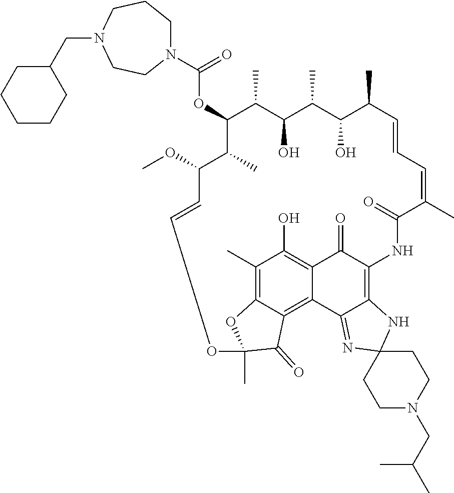 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(CC3CCCCC3)CC2)[C@@H]1C)C4=O] |
| 42 | 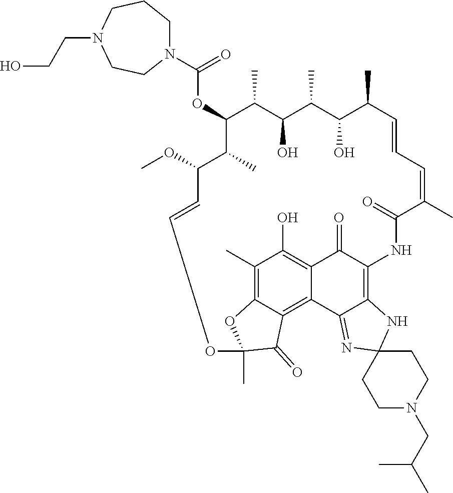 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(CCO)CC2)[C@@H]1C)C4=O] |
| 43 | 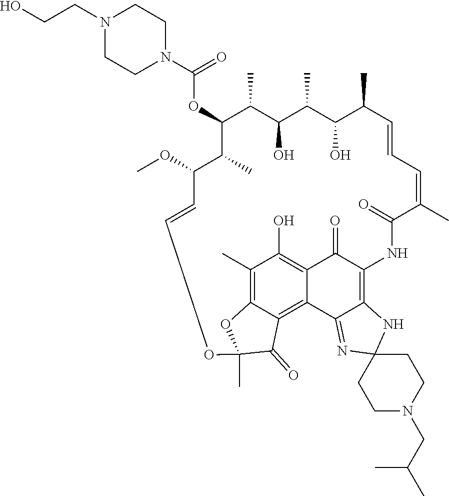 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCO)CC2)[C@@H]1C)C4=O] |
| 44 | 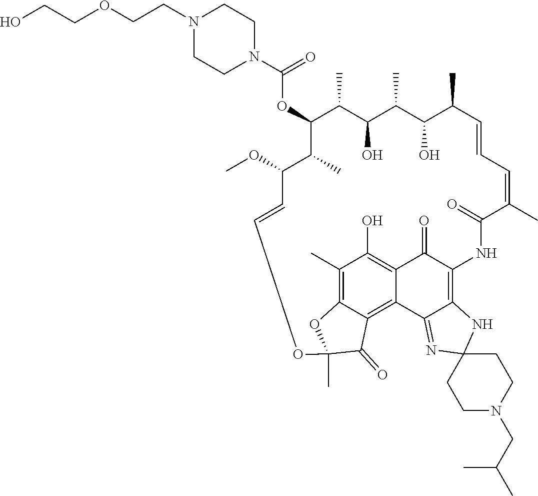 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCOCCO)CC2)[C@@H]1C)C4=O] |
| 45 | 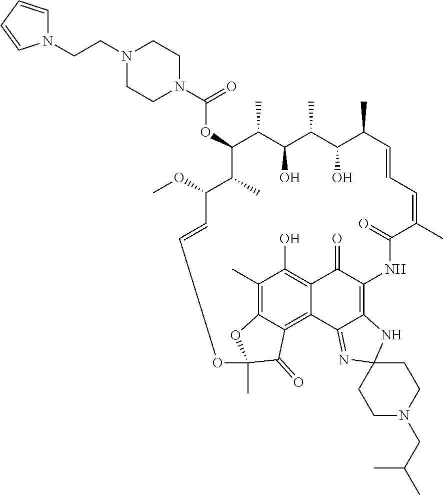 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCn3cccc3)CC2)[C@@H]1C)C4=O] |
| 46 | 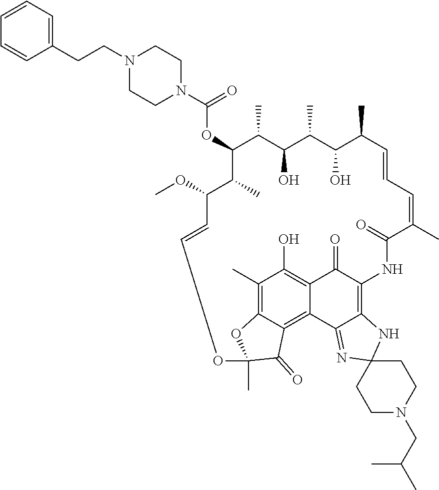 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCc3ccccc3)CC2)[C@@H]1C)C4=O] |
| 47 | 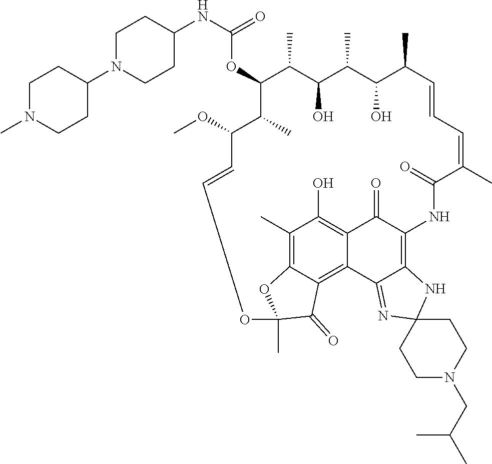 |
| 48 | 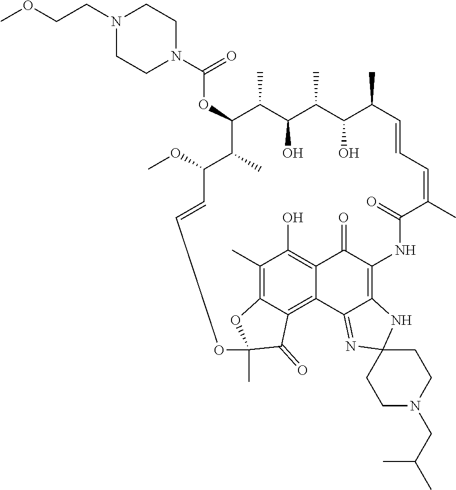 |
| 49 | 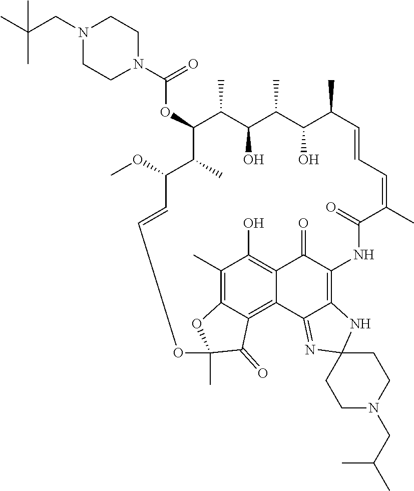 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC(C)(C)C)CC2)[C@@H]1C)C4=O] |
| 50 | 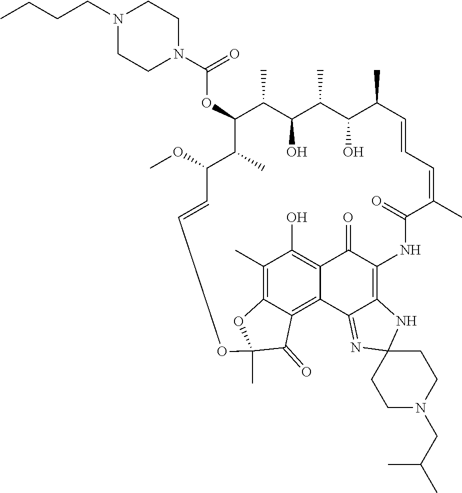 [SMILES: CCCCN1CCN(C(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1] |
| 51 | 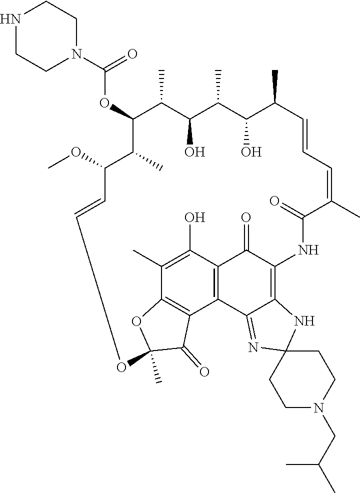 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCNCC2)[C@@H]1C)C4=O] |
| 52 | 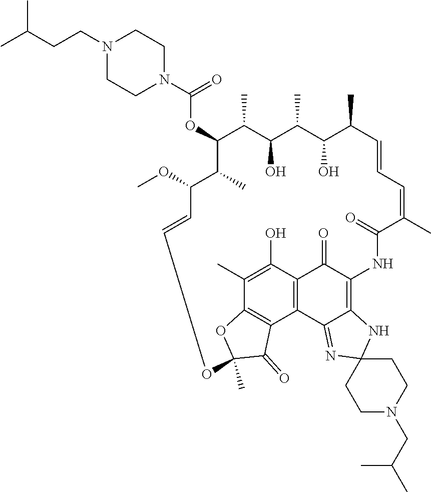 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCC(C)C)CC2)[C@@H]1C)C4=O] |
| 53 | 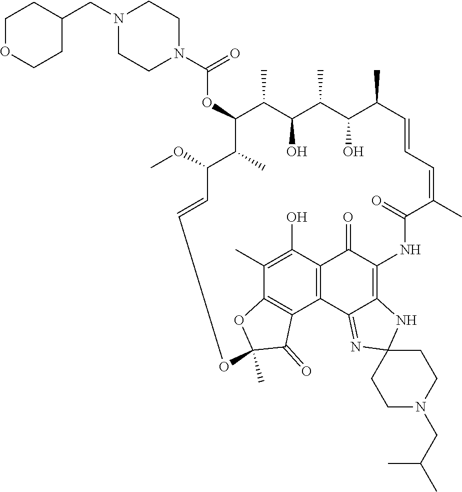 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCOCC3)CC2)[C@@H]1C)C4=O] |
| 54 | 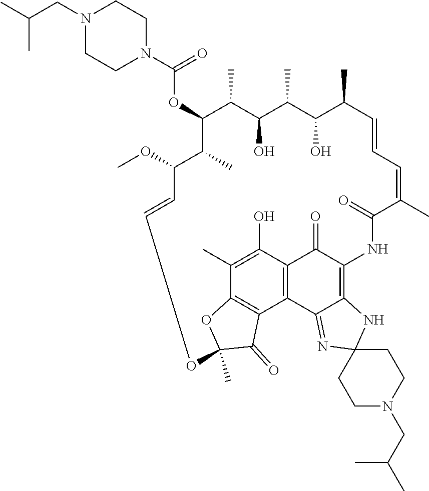 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC(C)C)CC2)[C@@H]1C)C4=O] |
| 55 | 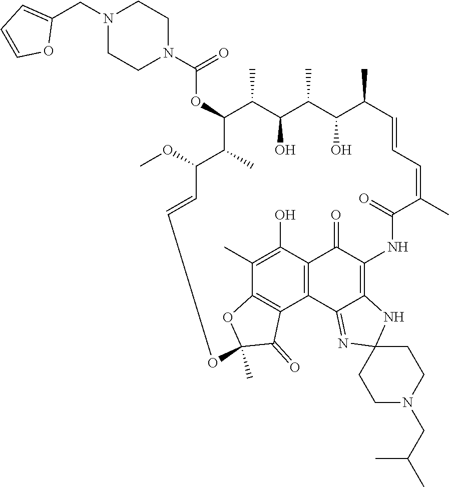 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccco3)CC2)[C@@H]1C)C4=O] |
| 56 | 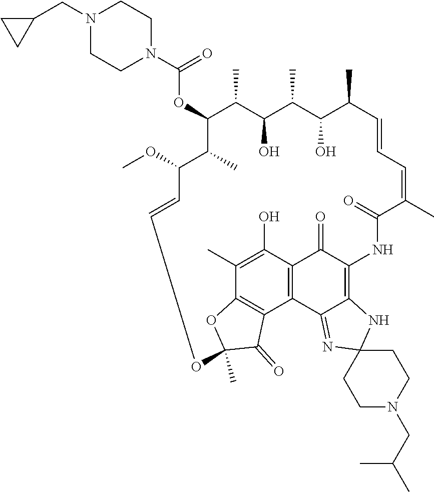 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CC3)CC2)[C@@H]1C)C4=O] |
| 57 | 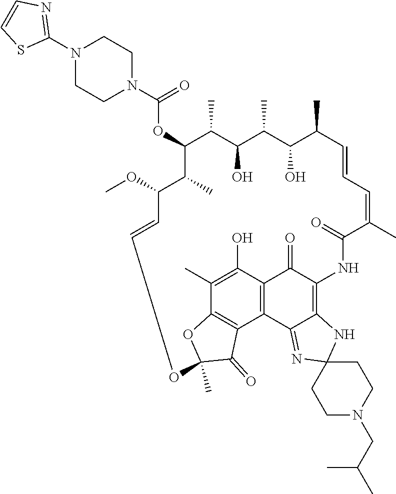 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3nccs3)CC2)[C@@H]1C)C4=O] |
| 58 | 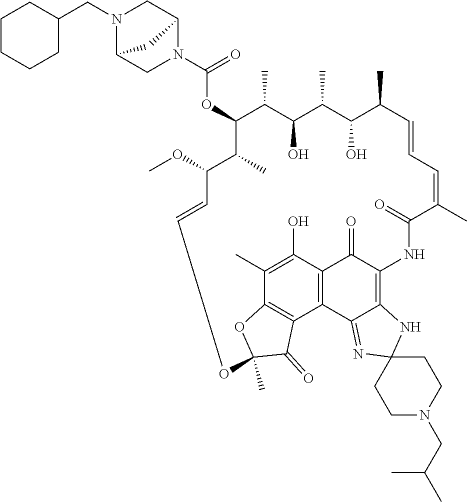 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@@H]3C[C@H]2CN3CC2CCCCC2)[C@@H]1C)C4=O] |
| 59 | 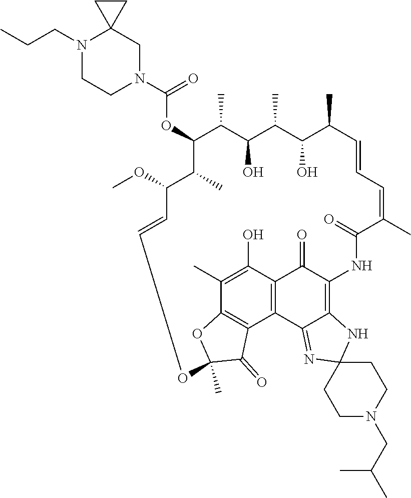 [SMILES: CCCN1CCN(C(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC12CC2] |
| 60 | 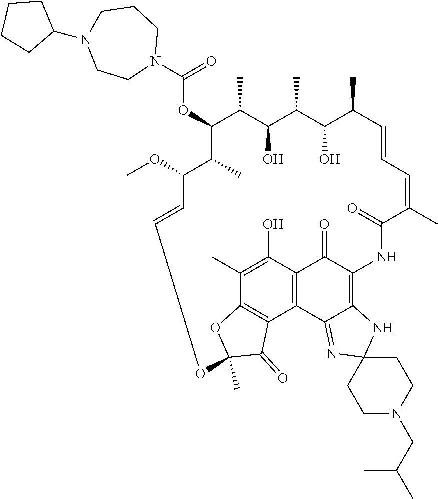 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(C3CCCC3)CC2)[C@@H]1C)C4=O] |
| 61 | 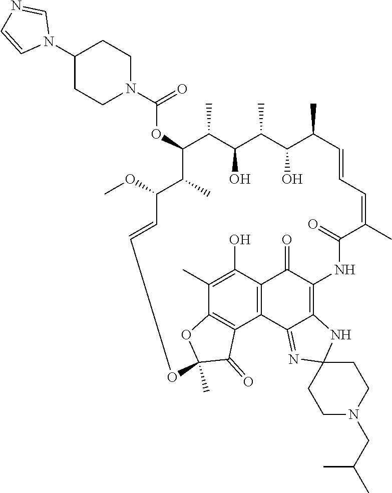 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(n3ccnc3)CC2)[C@@H]1C)C4=O] |
| 62 | 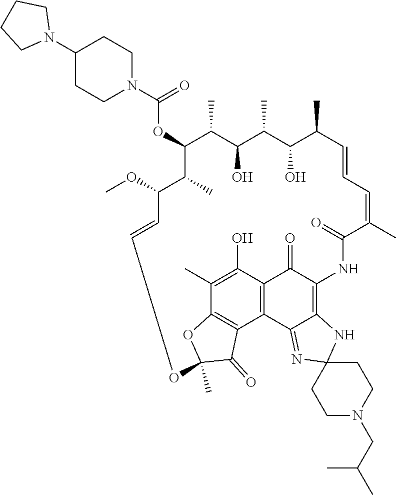 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(N3CCCC3)CC2)[C@@H]1C)C4=O] |
| 63 | 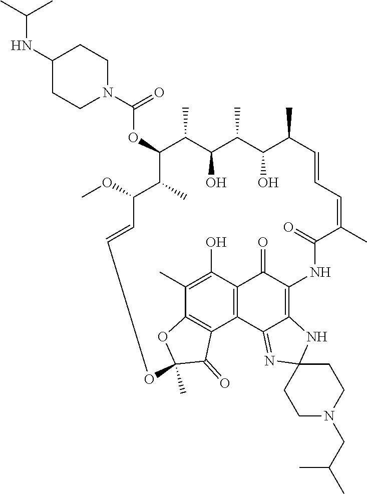 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(NC(C)C)CC2)[C@@H]1C)C4=O] |
| 64 | 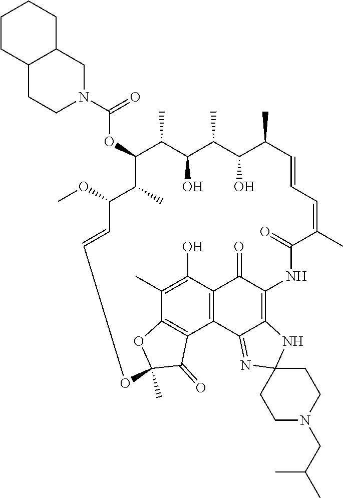 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3CCCCC3C2)[C@@H]1C)C4=O] |
| 65 | 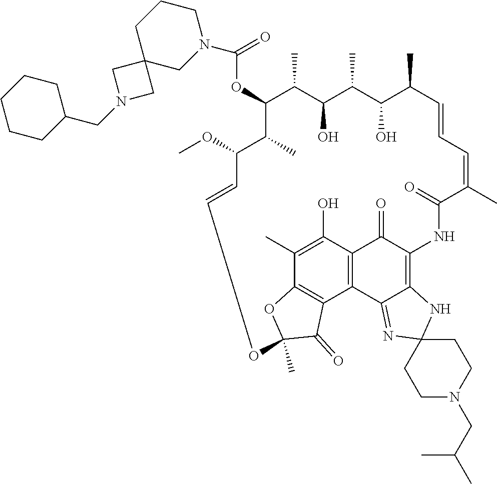 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CN(CC5CCCCC5)C3)C2)[C@@H]1C)C4=O] |
| 66 | 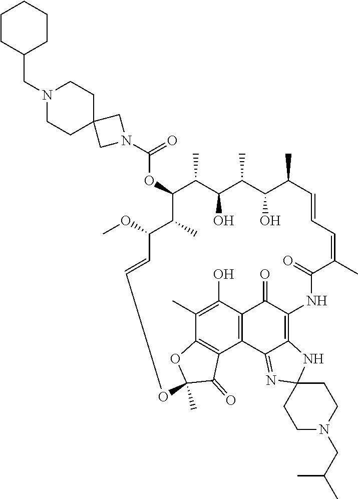 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3(CCN(CC5CCCCC5)CC3)C2)[C@@H]1C)C4=O] |
| 67 | 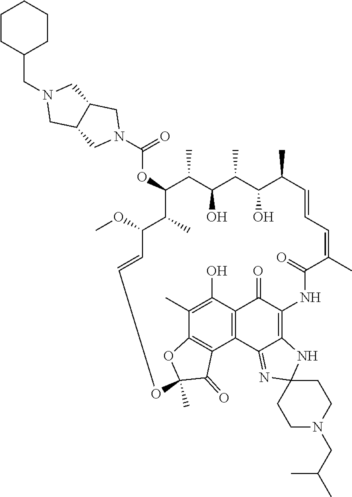 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@H]3CN(CC5CCCCC5)C[C@H]3C2)[C@@H]1C)C4=O] |
| 68 | 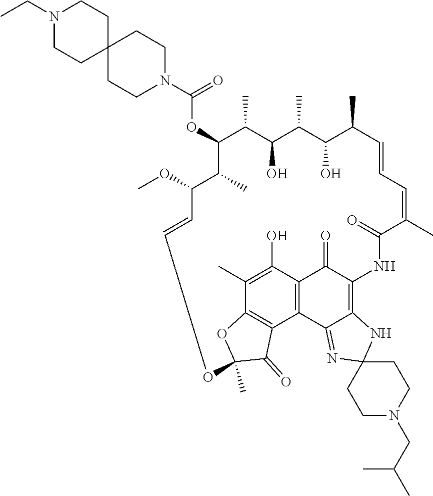 [SMILES: CCN1CCC2(CC1)CCN(C(=O)O[C@H]1[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]1C)O5)C3=O)CC2] |
| 69 | 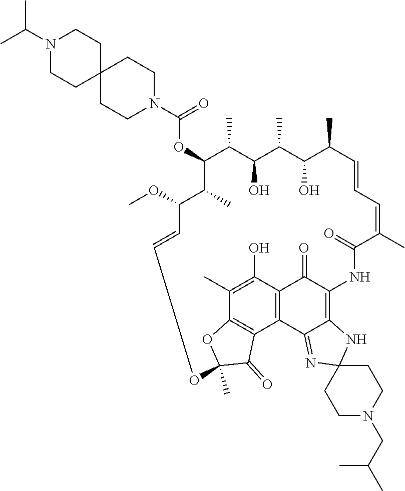 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CC2)CCN(C(C)C)CC3)[C@@H]1C)C4=O] |
| 70 | 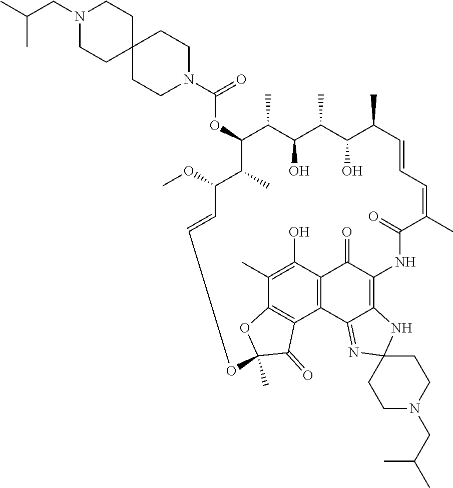 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC(C)C)CC3)CC2)[C@@H]1C)C4=O] |
| 71 | 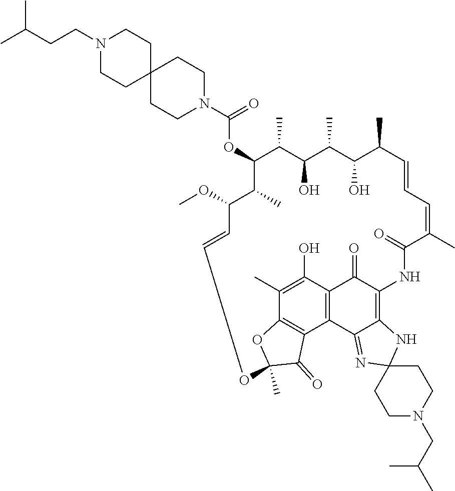 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CCC(C)C)CC3)CC2)[C@@H]1C)C4=O] |
| 72 | 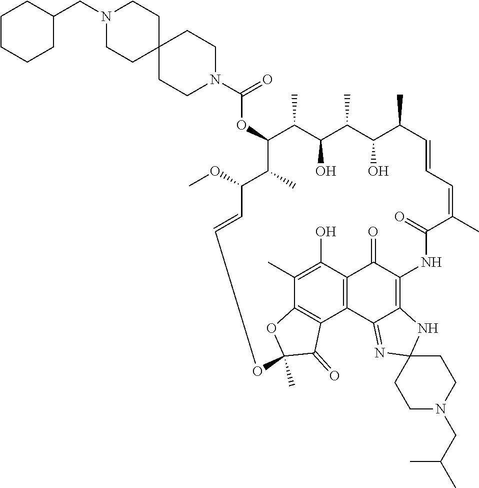 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC5CCCCC5)CC3)CC2)[C@@H]1C)C4=O] |
| 73 | 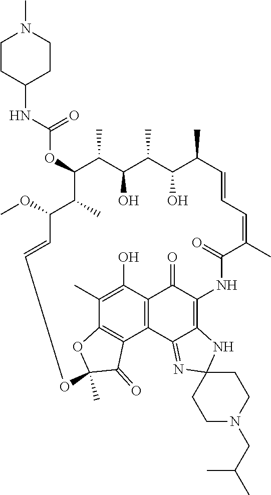 |
| 74 | 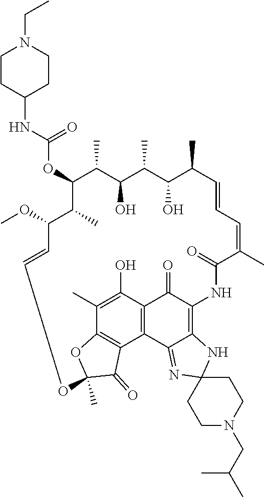 |
| 75 | 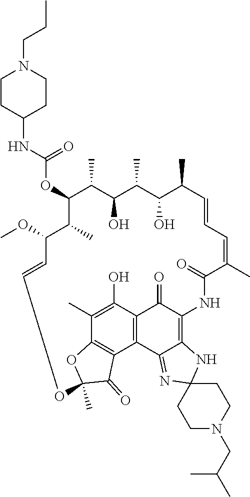 |
| 76 | 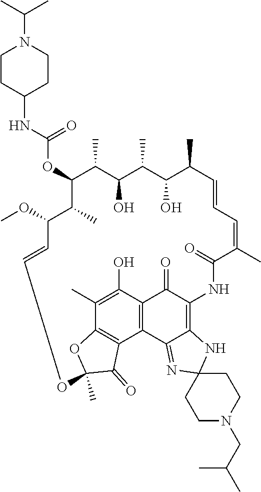 |
| 77 | 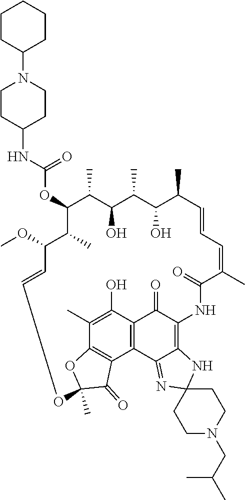 |
| 78 | 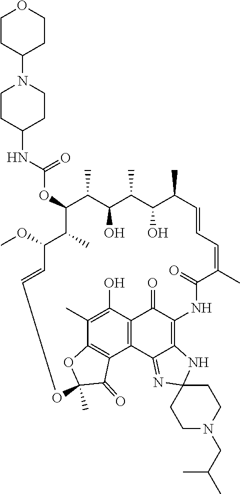 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(C3CCOCC3)CC2)[C@@H]1C)C4=O] |
| 79 | 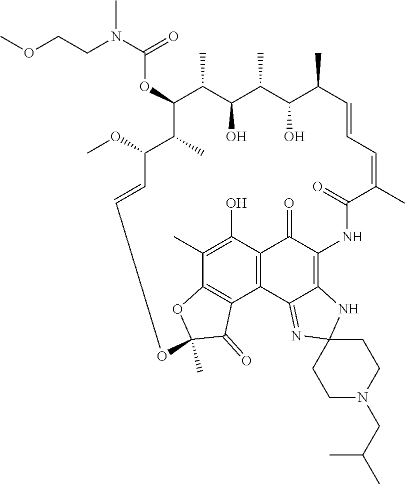 |
| 80 | 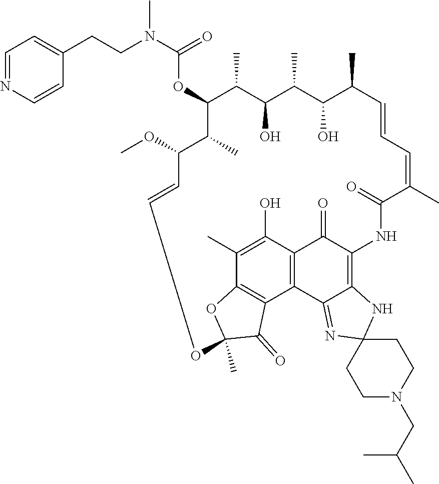 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)CCc2ccncc2)[C@@H]1C)C4=O] |
| 81 | 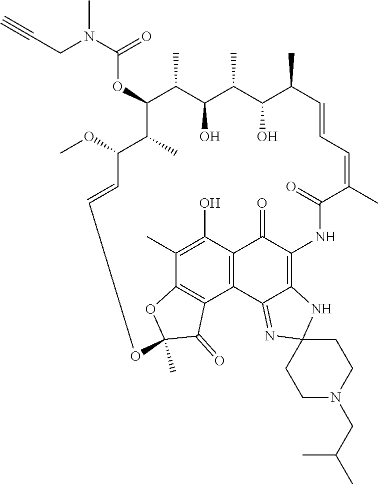 [SMILES: C#CCN(C)C(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] |
| 82 | 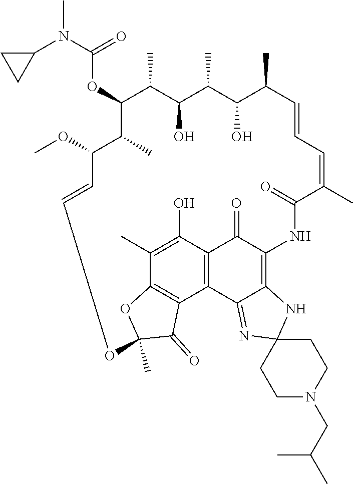 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)C2CC2)[C@@H]1C)C4=O] |
| 83 | 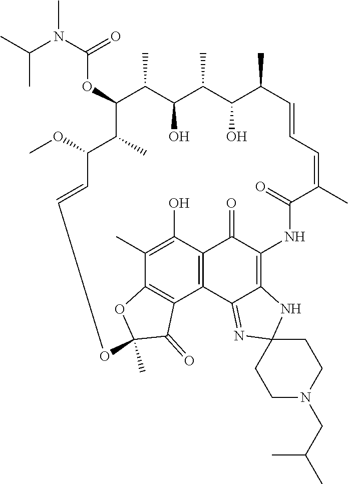 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)C(C)C)[C@@H]1C)C4=O] |
| 84 | 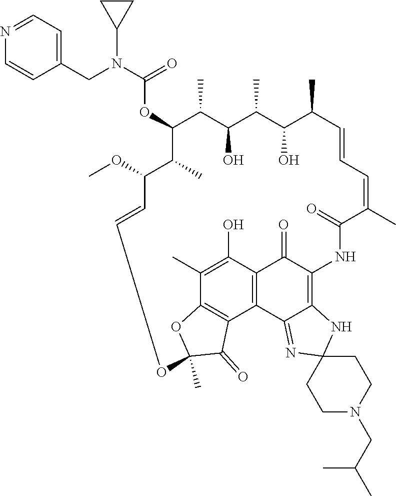 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(Cc2ccncc2)C2CC2)[C@@H]1C)C4=O] |

In some embodiments, the compound of Formula I is selected from the group consisting of the compounds shown in Table 3. In some embodiments of any of the above aspects, the compound of Formula I is selected from Table 3, below:

TABLE 3
Selected Compounds of the Invention

| Cpd | Structure |
|---|---|
| 1 | 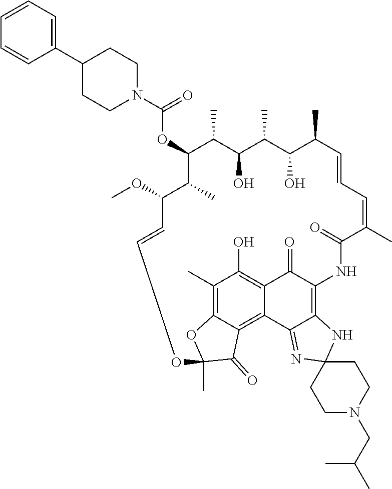 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccccc3)CC2)[C@@H]1C)C4=O] |
| 2 | 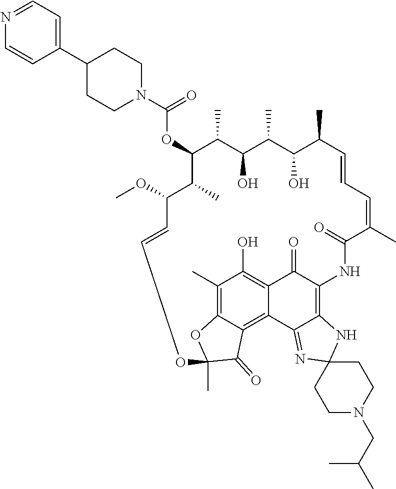 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccncc3)CC2)[C@@H]1C)C4=O] |
| 3 | 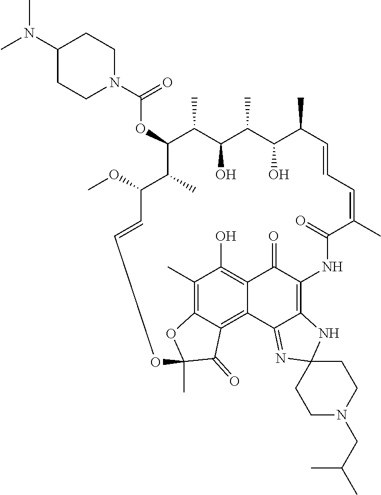 |
| 4 | 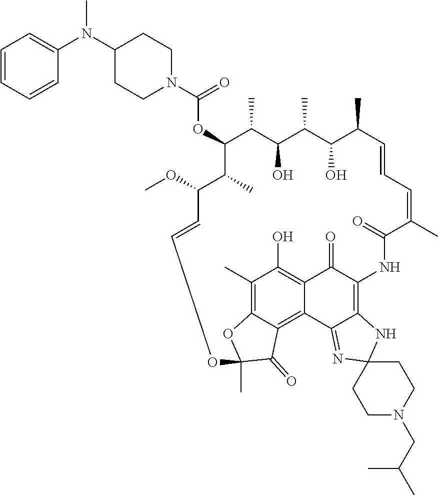 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(N(C)c3ccccc3)CC2)[C@@H]1C)C4=O] |
| 5 | 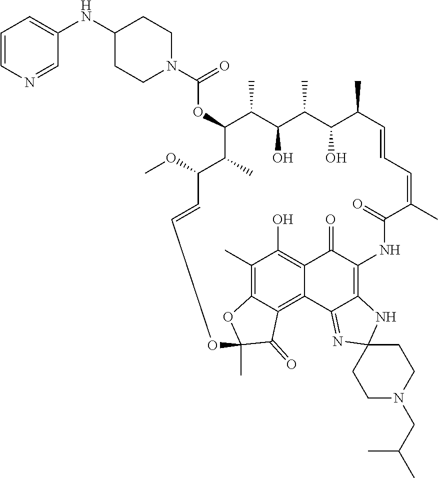 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(Nc3cccnc3)CC2)[C@@H]1C)C4=O] |
| 6 | 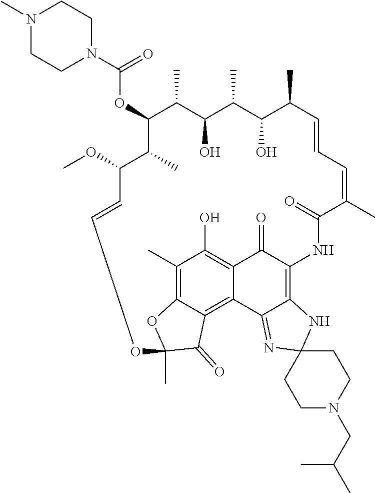 |
| 7 | 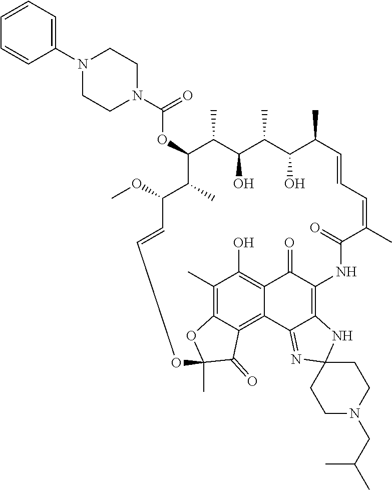 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccc3)CC2)[C@@H]1C)C4=O] |
| 8 | 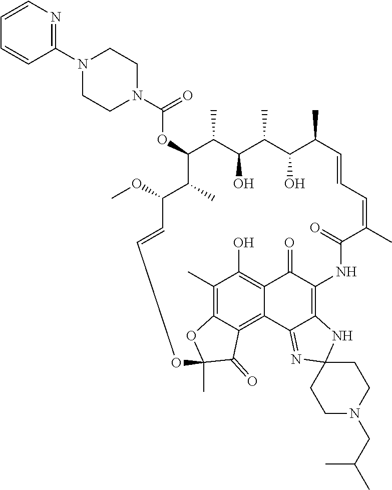 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccn3)CC2)[C@@H]1C)C4=O] |
| 9 | 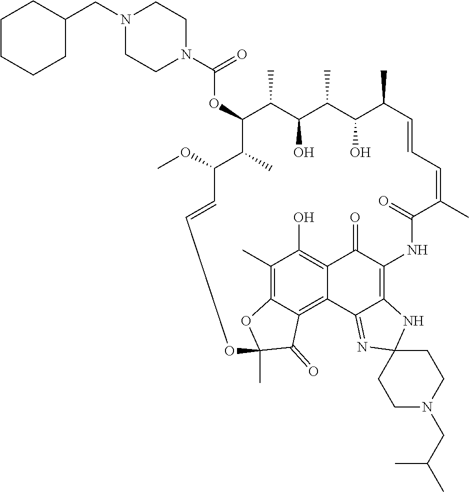 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCCC3)CC2)[C@@H]1C)C4=O] |
| 10 | 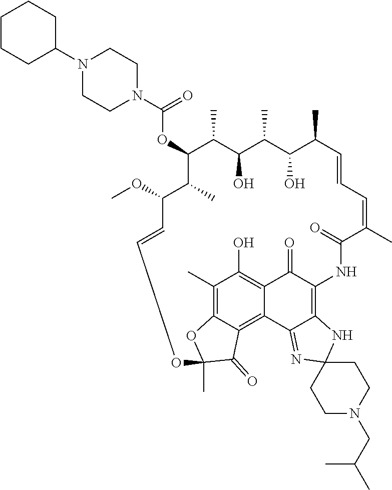 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCCCC3)CC2)[C@@H]1C)C4=O] |
| 11 | 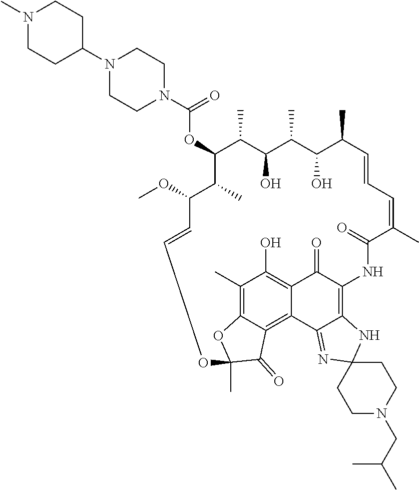 |
| 12 | 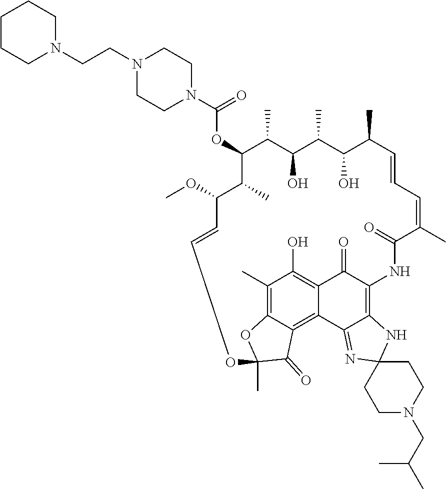 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCCCC3)CC2)[C@@H]1C)C4=O] |
| 13 | 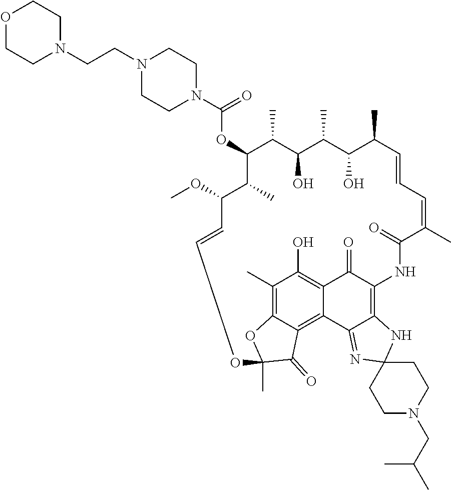 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCOCC3)CC2)[C@@H]1C)C4=O] |
| 14 | 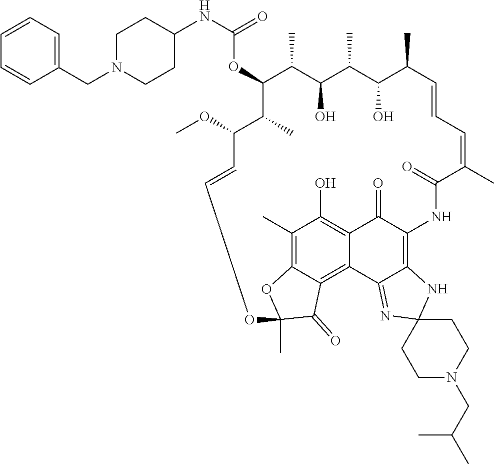 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)CC2)[C@@H]1C)C4=O] |
| 15 | 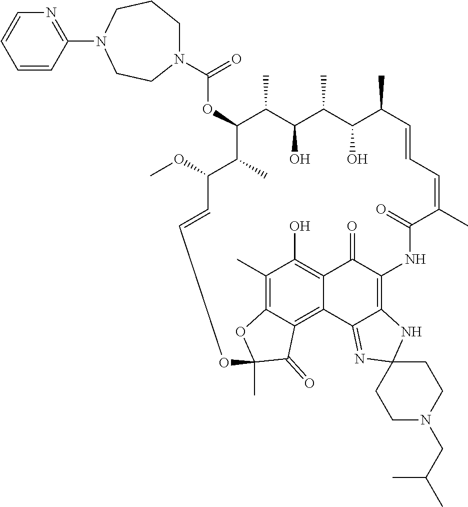 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccccn3)CC2)[C@@H]1C)C4=O] |
| 16 | 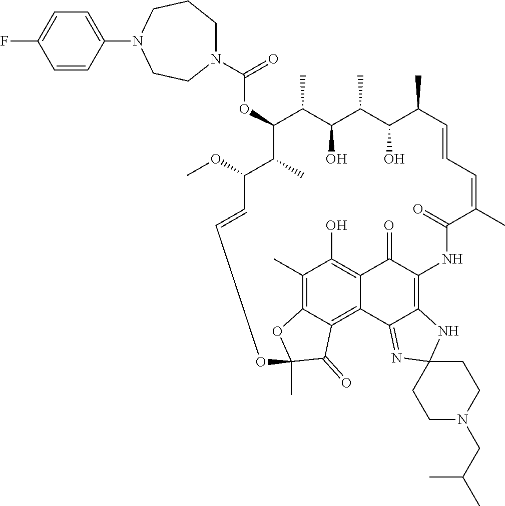 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccc(F)cc3)CC2)[C@@H]1C)C4=O] |
| 17 | 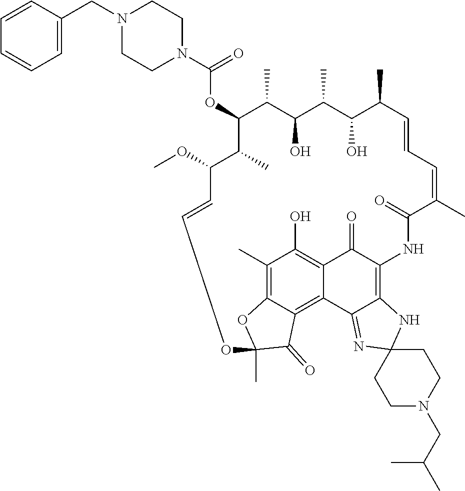 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccccc3)CC2)[C@@H]1C)C4=O] |
| 20 | 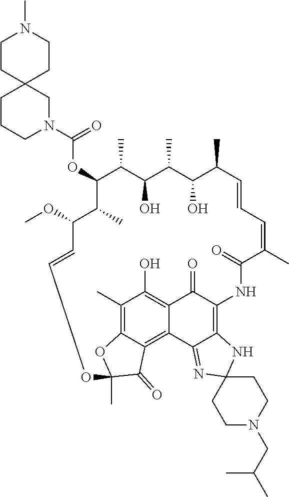 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CCN(C)CC3)C2)[C@@H]1C)C4=O] |
| 21 | 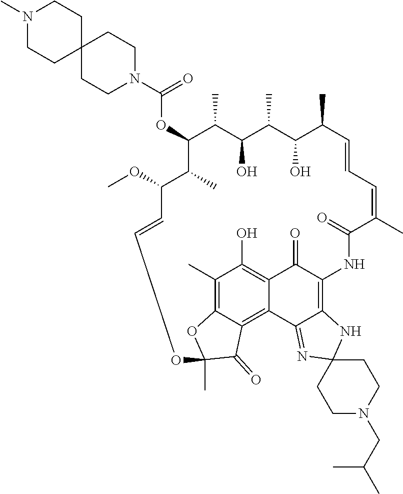 |
| 24 | 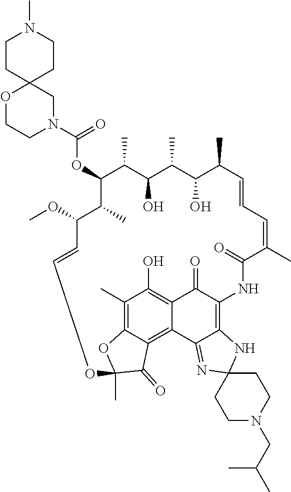 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCOC3(CCN(C)CC3)C2)[C@@H]1C)C4=O] |
| 25 | 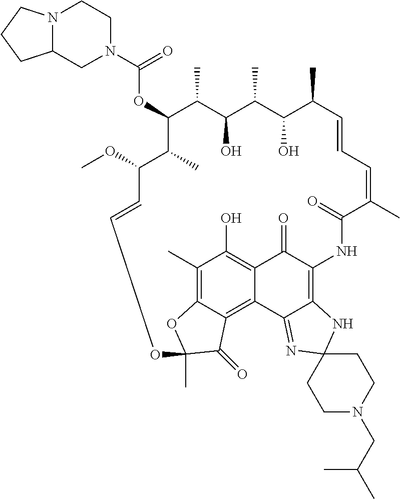 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN3CCCC3C2)[C@@H]1C)C4=O] |
| 29 | 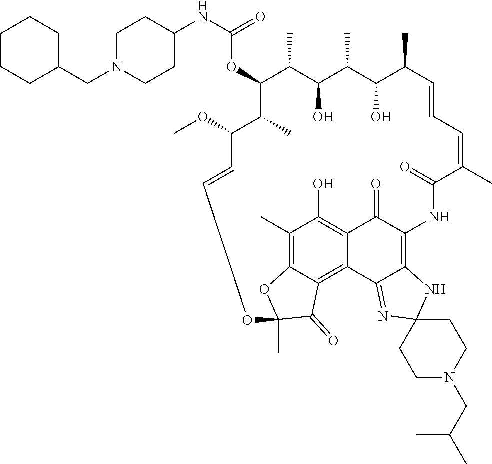 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(CC3CCCCC3)CC2)[C@@H]1C)C4=O] |
| 30 | 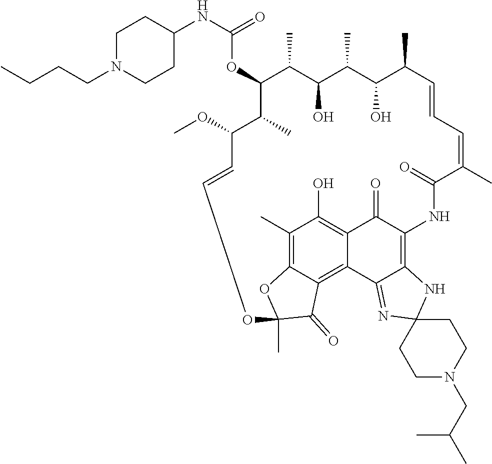 [SMILES: CCCCN1CCC(NC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1] |
| 32, 32a | 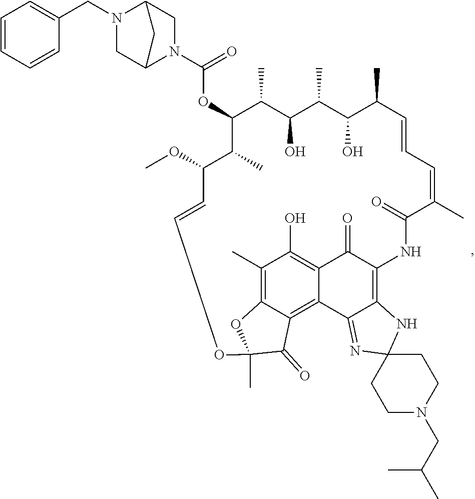 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3CC2CN3Cc2ccccc2)[C@@H]1C)C4=O] |
| | preferably |
| | 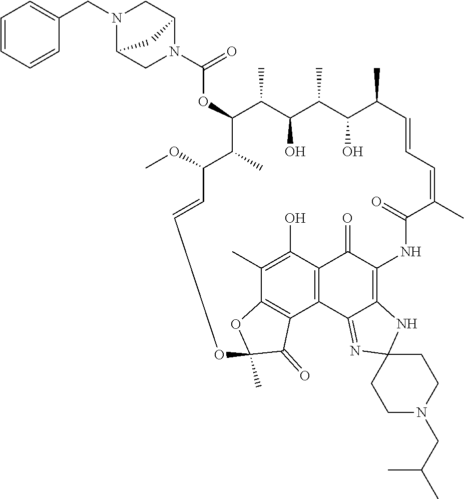 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@@H]3C[C@H]2CN3Cc2ccccc2)[C@@H]1C)C4=O] |
| 36 | 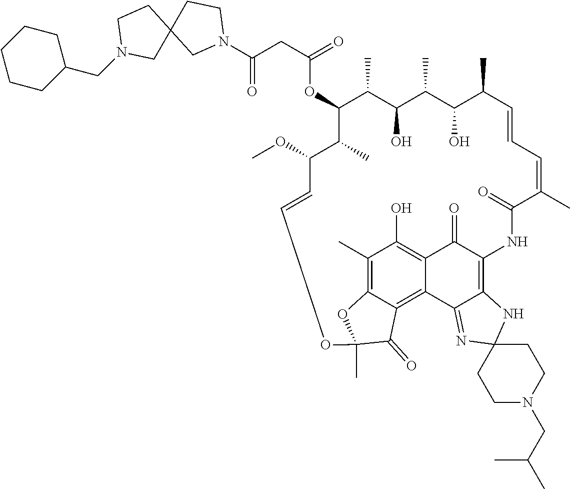 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CC(=O)N2CCC3(CCN(CC5CCCCC5)C3)C2)[C@@H]1C)C4=O] |
| 37 | 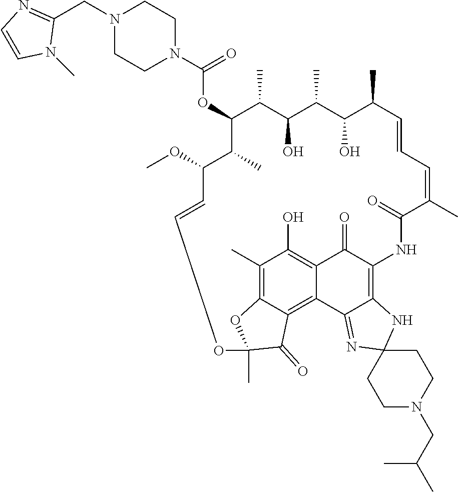 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3nccn3C)CC2)[C@@H]1C)C4=O] |
| 38 | 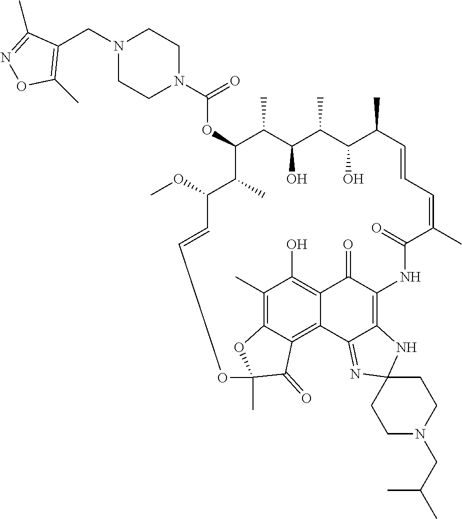 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3c(C)noc3C)CC2)[C@@H]1C)C4=O] |
| 41 | 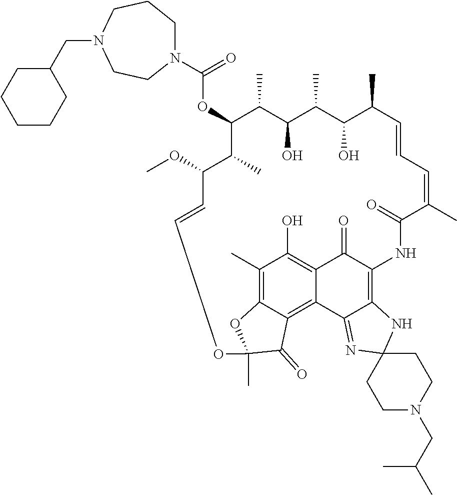 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(CC3CCCCC3)CC2)[C@@H]1C)C4=O] |
| 43 | 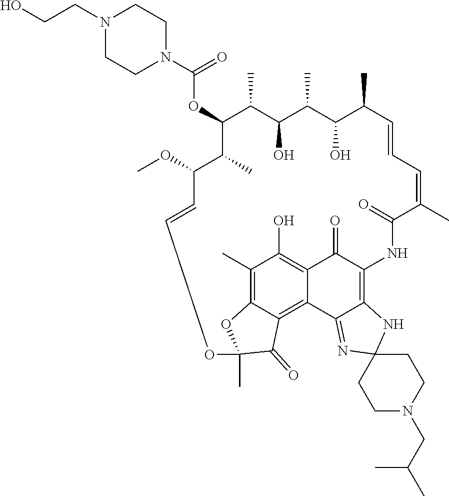 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCO)CC2)[C@@H]1C)C4=O] |
| 44 | 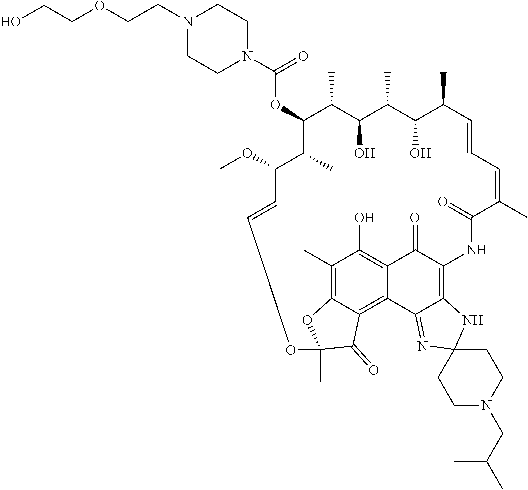 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCOCCO)CC2)[C@@H]1C)C4=O] |
| 46 | 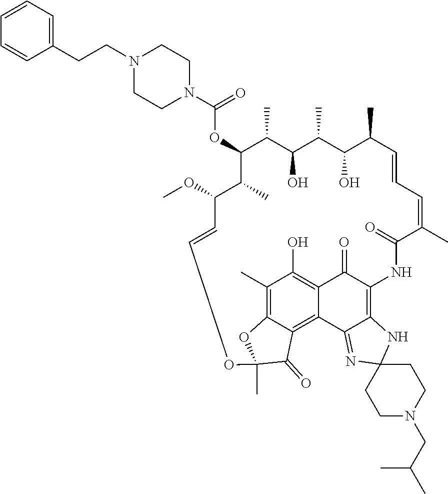 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCc3ccccc3)CC2)[C@@H]1C)C4=O] |
| 48 | 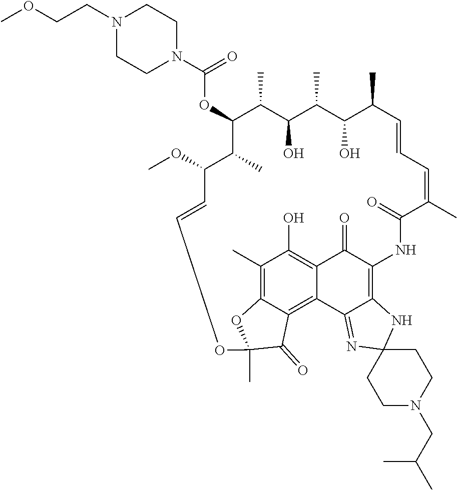 |
| 49 | 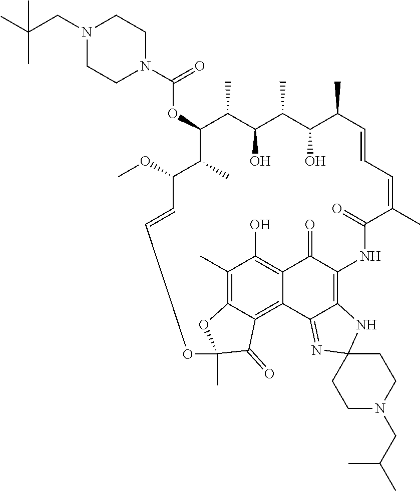 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC(C)(C)C)CC2)[C@@H]1C)C4=O] |
| 50 | 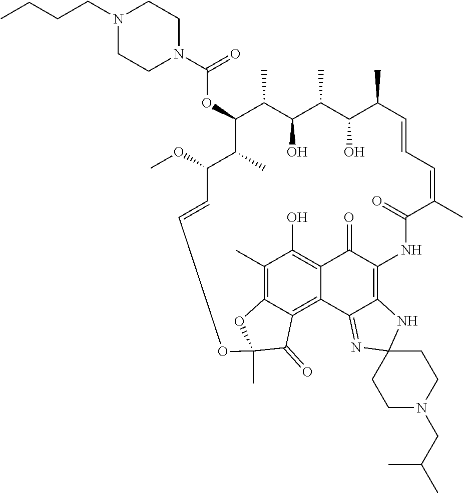 [SMILES: CCCCN1CCN(C(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1] |
| 51 | 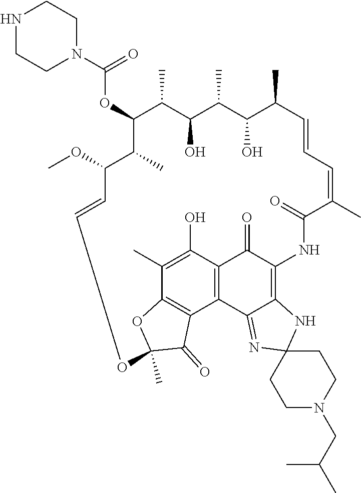 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCNCC2)[C@@H]1C)C4=O] |
| 52 | 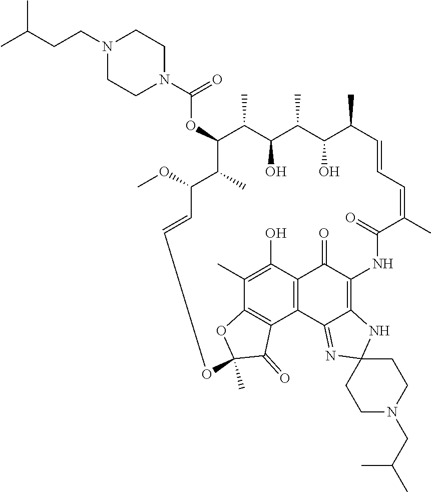 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCC(C)C)CC2)[C@@H]1C)C4=O] |
| 53 | 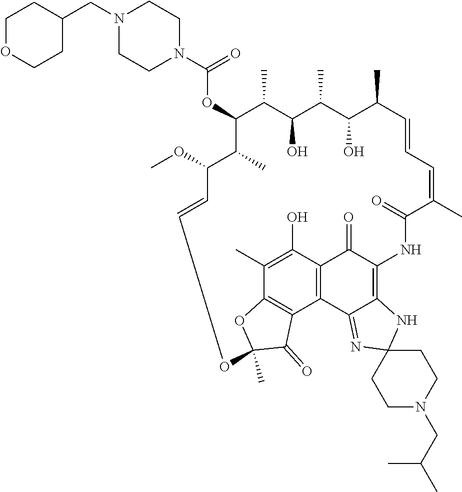 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCOCC3)CC2)[C@@H]1C)C4=O] |
| 54 | 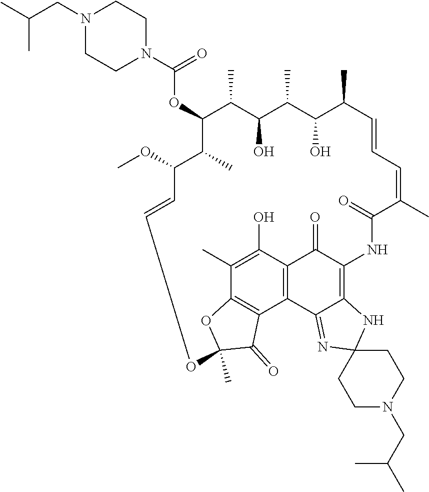 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC(C)C)CC2)[C@@H]1C)C4=O] |
| 55 | 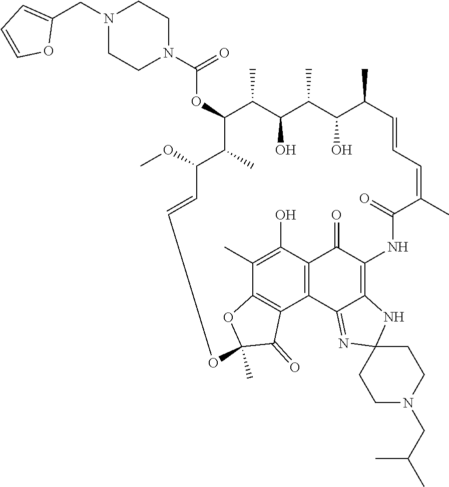 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccco3)CC2)[C@@H]1C)C4=O] |
| 56 | 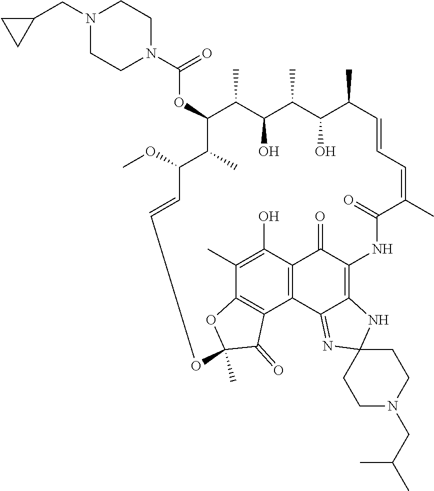 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CC3)CC2)[C@@H]1C)C4=O] |
| 57 | 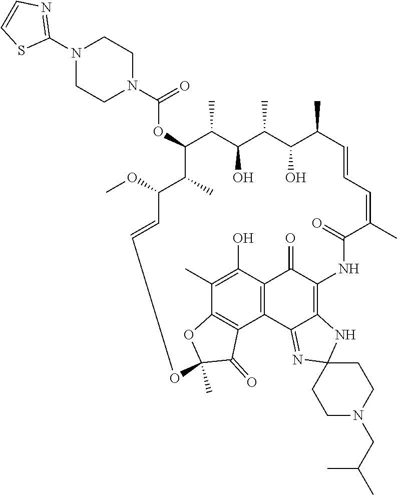 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3nccs3)CC2)[C@@H]1C)C4=O] |
| 58 | 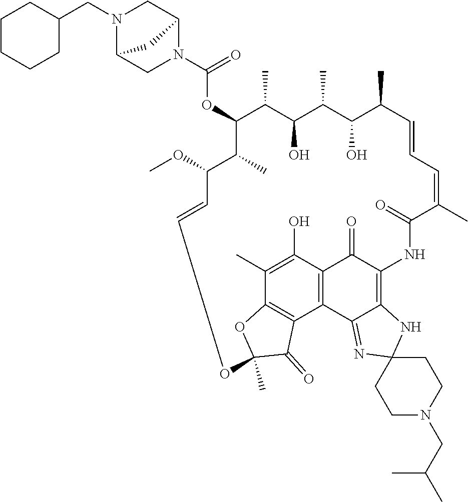 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@@H]3C[C@H]2CN3CC2CCCCC2)[C@@H]1C)C4=O] |
| 59 | 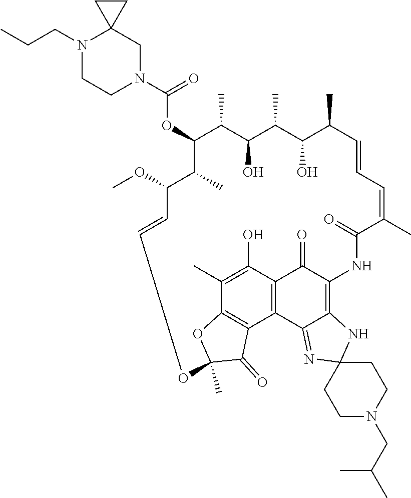 [SMILES: CCCN1CCN(C(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC12CC2] |
| 60 | 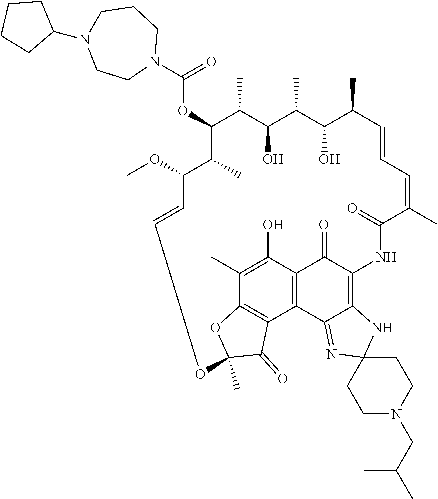 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(C3CCCC3)CC2)[C@@H]1C)C4=O] |
| 61 | 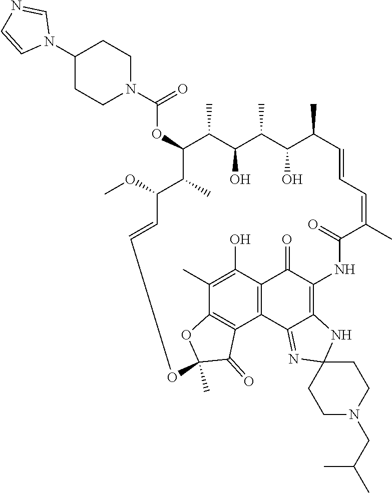 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(n3ccnc3)CC2)[C@@H]1C)C4=O] |
| 62 | 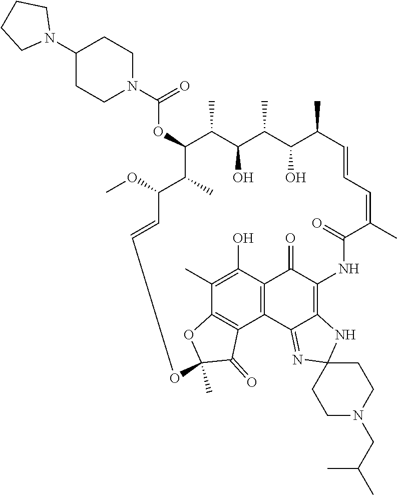 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(N3CCCC3)CC2)[C@@H]1C)C4=O] |
| 63 | 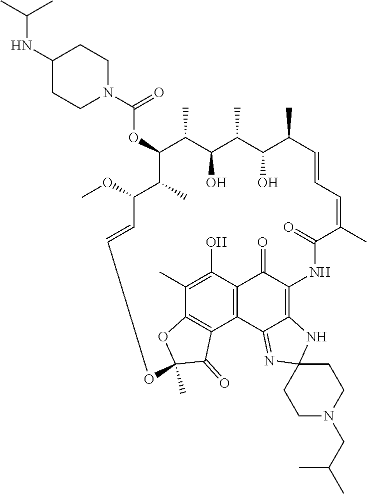 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(NC(C)C)CC2)[C@@H]1C)C4=O] |
| 64 | 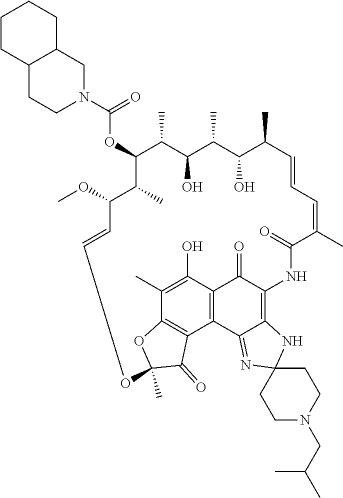 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3CCCCC3C2)[C@@H]1C)C4=O] |
| 65 | 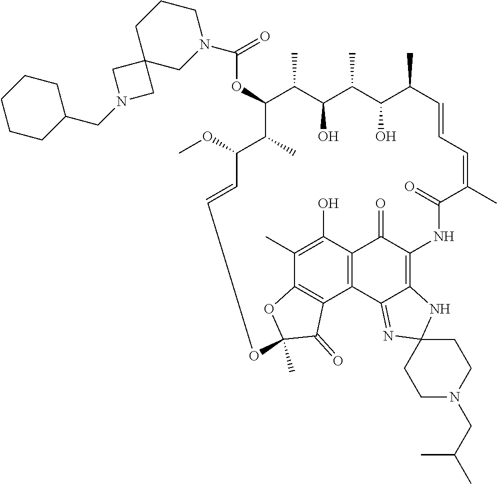 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CN(CC5CCCCC5)C3)C2)[C@@H]1C)C4=O] |
| 66 | 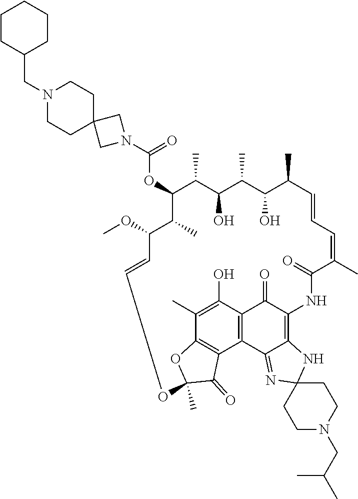 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3(CCN(CC5CCCCC5)CC3)C2)[C@@H]1C)C4=O] |
| 67 | 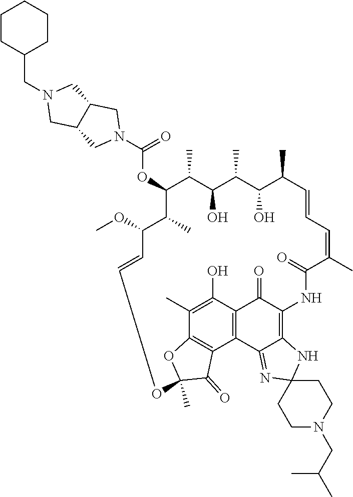 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@H]3CN(CC5CCCCC5)C[C@H]3C2)[C@@H]1C)C4=O] |
| 68 | 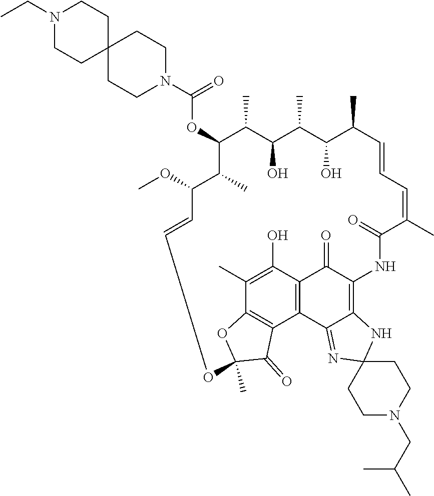 [SMILES: CCN1CCC2(CC1)CCN(C(=O)O[C@H]1[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]1C)O5)C3=O)CC2] |
| 69 | 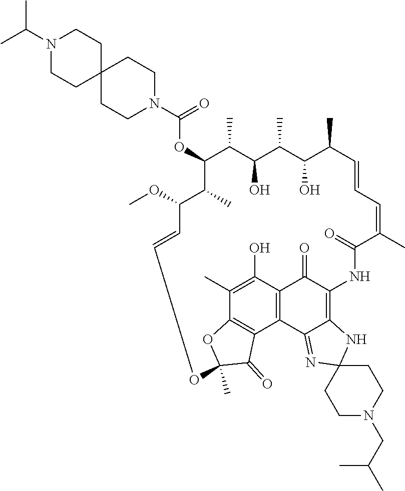 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CC2)CCN(C(C)C)CC3)[C@@H]1C)C4=O] |
| 70 | 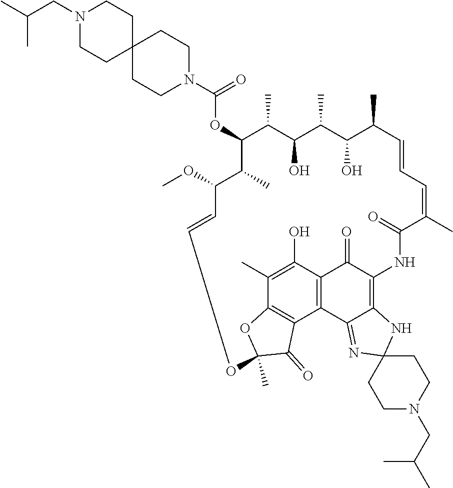 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC(C)C)CC3)CC2)[C@@H]1C)C4=O] |
| 71 | 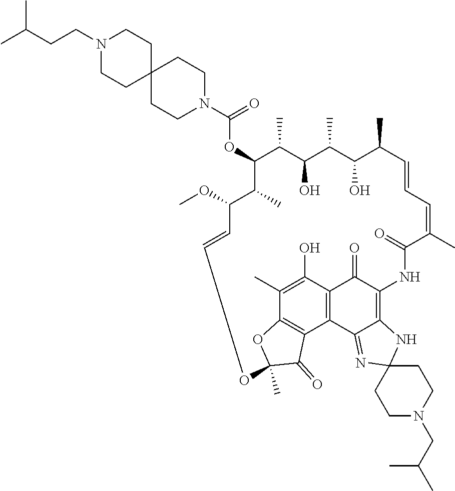 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CCC(C)C)CC3)CC2)[C@@H]1C)C4=O] |
| 72 | 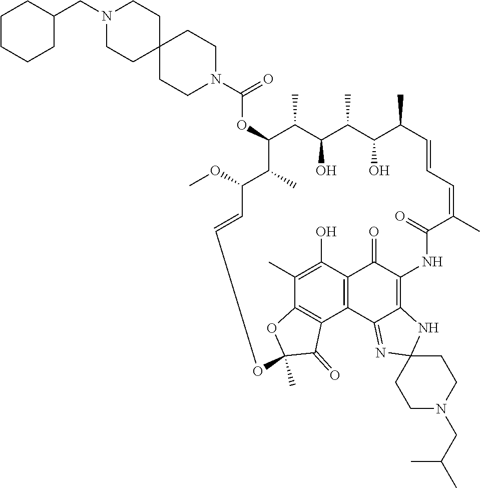 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC5CCCCC5)CC3)CC2)[C@@H]1C)C4=O] |
| 73 | 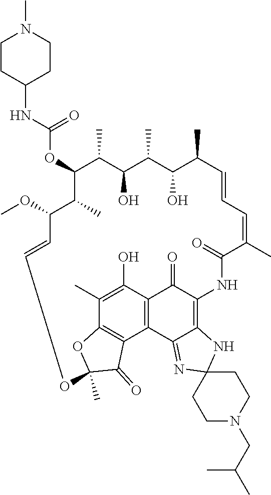 |
| 74 | 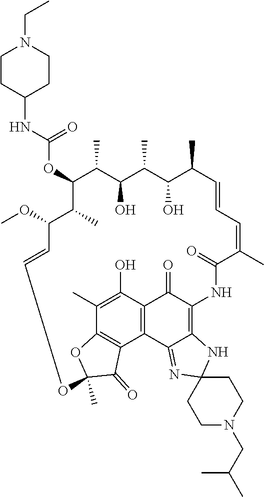 |
| 75 | 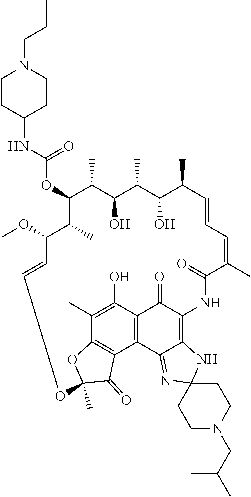 |
| 76 | 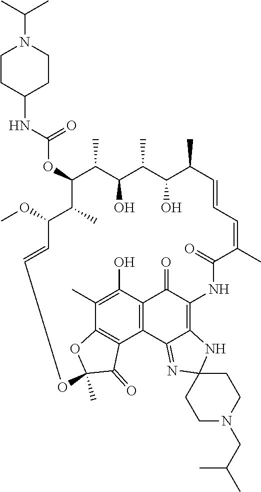 |
| 77 | 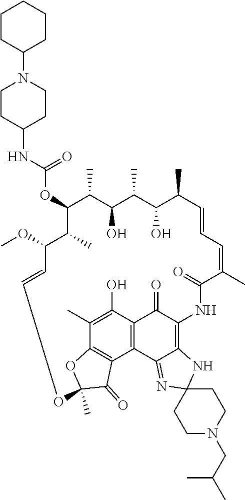 |
| 78 | 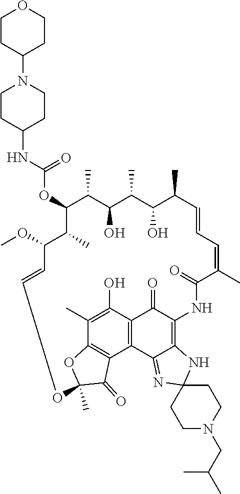 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(C3CCOCC3)CC2)[C@@H]1C)C4=O] |
| 82 | 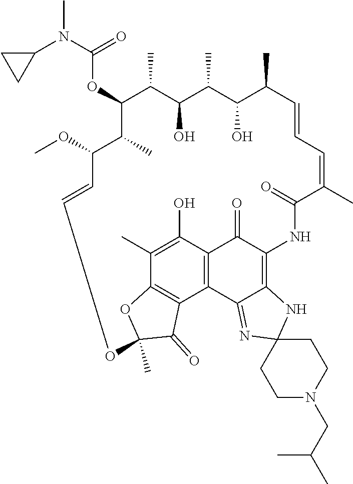 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)C2CC2)[C@@H]1C)C4=O] |
| 83 | 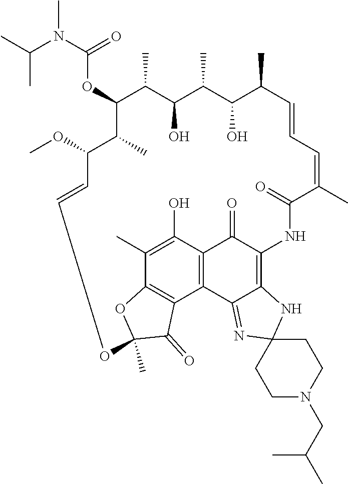 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)C(C)C)[C@@H]1C)C4=O] |

In some embodiments, the compound of Formula I is selected from the group consisting of the compounds shown in Table 4. In some embodiments of any of the above aspects, the compound of Formula I is selected from Table 4, below:

TABLE 4
Selected Compounds of the Invention

| Cpd | Structure |
|---|---|
| 1 | 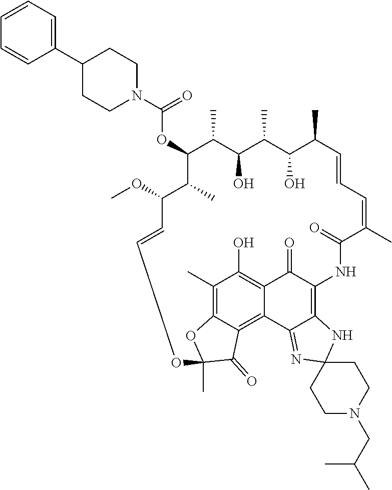 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccccc3)CC2)[C@@H]1C)C4=O] |
| 2 | 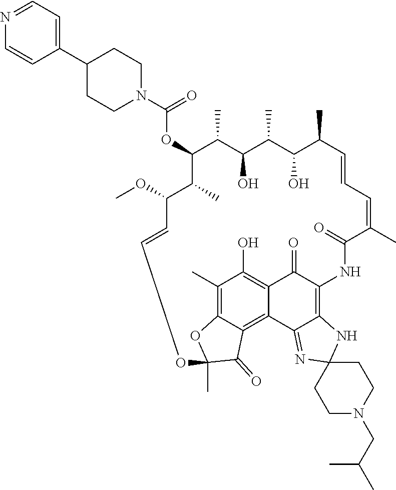 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccncc3)CC2)[C@@H]1C)C4=O] |
| 3 | 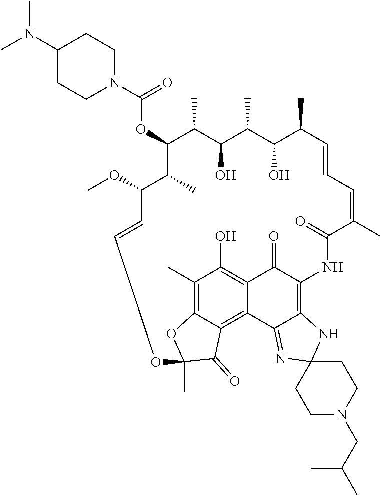 |
| 4 | 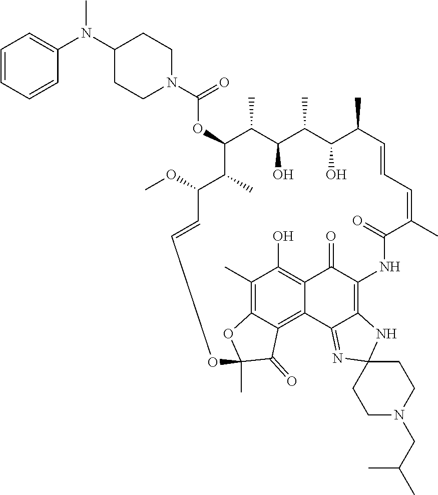 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(N(C)c3ccccc3)CC2)[C@@H]1C)C4=O] |
| 5 | 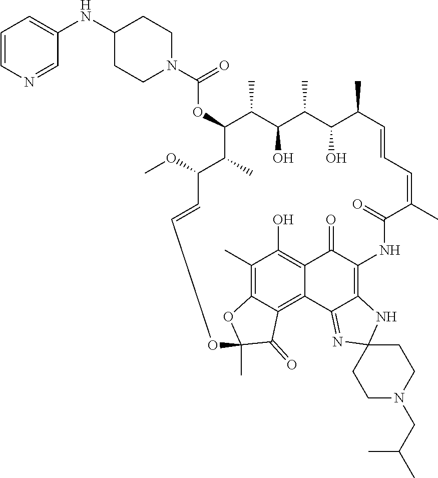 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(Nc3cccnc3)CC2)[C@@H]1C)C4=O] |
| 6 | 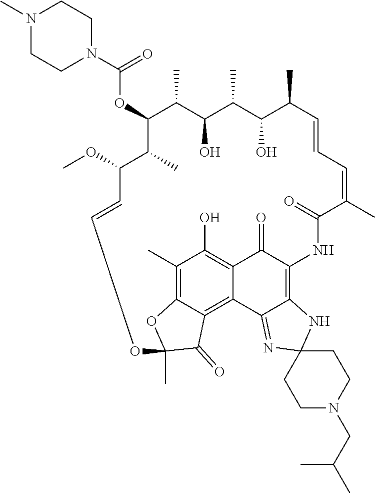 |
| 7 | 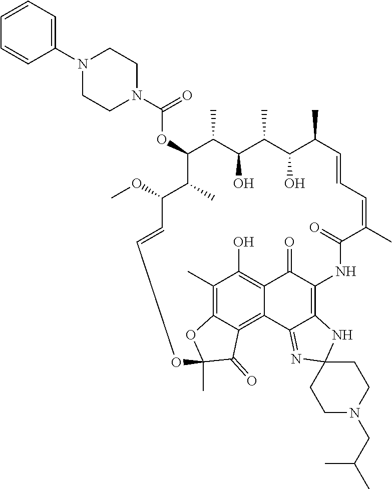 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccc3)CC2)[C@@H]1C)C4=O] |
| 8 | 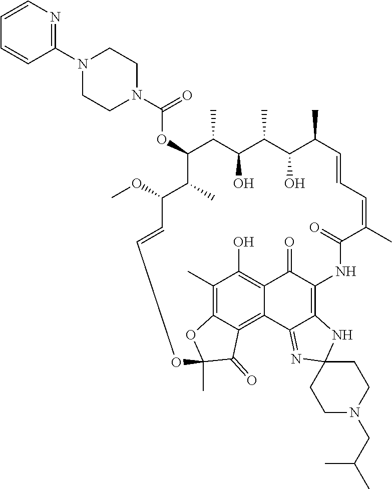 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccn3)CC2)[C@@H]1C)C4=O] |
| 9 | 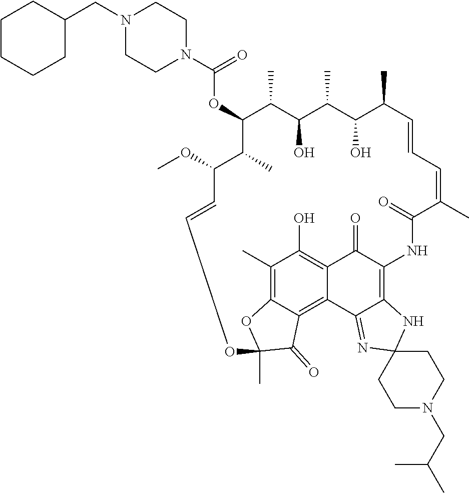 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCCC3)CC2)[C@@H]1C)C4=O] |
| 10 | 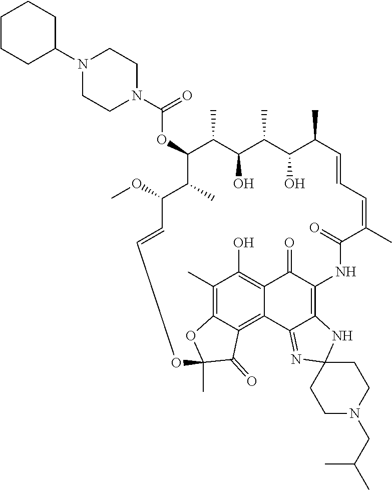 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCCCC3)CC2)[C@@H]1C)C4=O] |
| 11 | 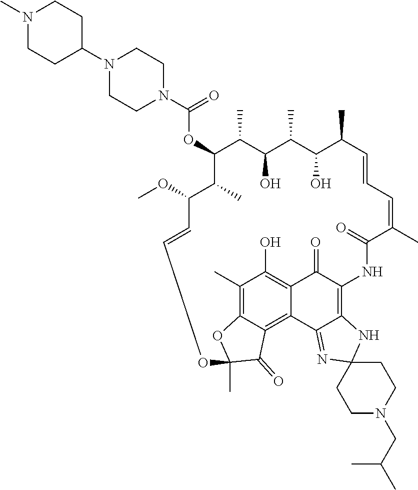 |
| 12 | 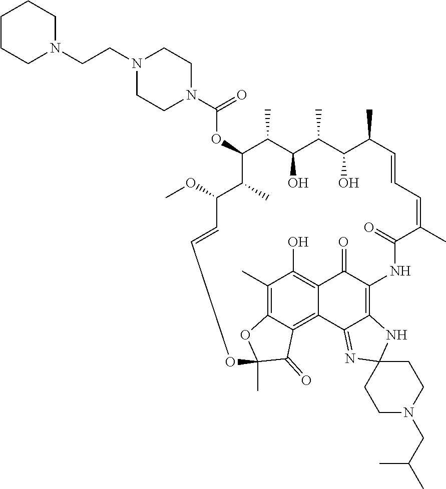 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCCCC3)CC2)[C@@H]1C)C4=O] |
| 13 | 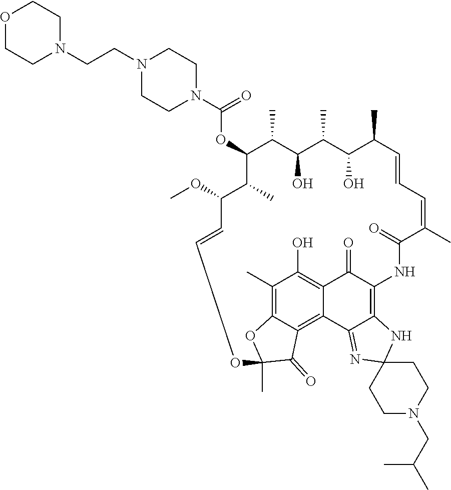 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCOCC3)CC2)[C@@H]1C)C4=O] |
| 14 | 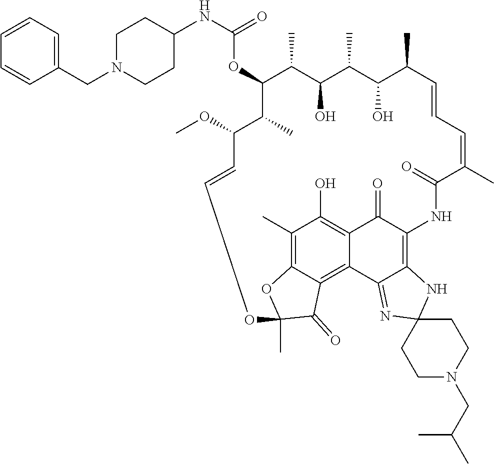 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)CC2)[C@@H]1C)C4=O] |
| 15 | 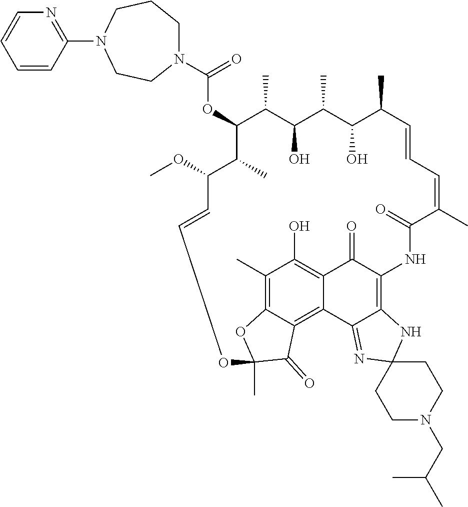 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccccn3)CC2)[C@@H]1C)C4=O] |
| 16 | 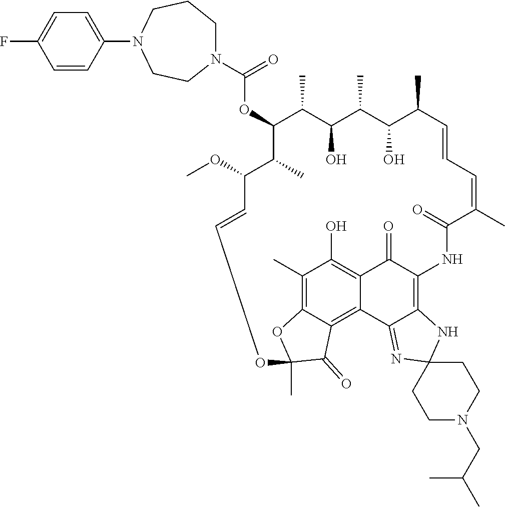 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccc(F)cc3)CC2)[C@@H]1C)C4=O] |
| 17 | 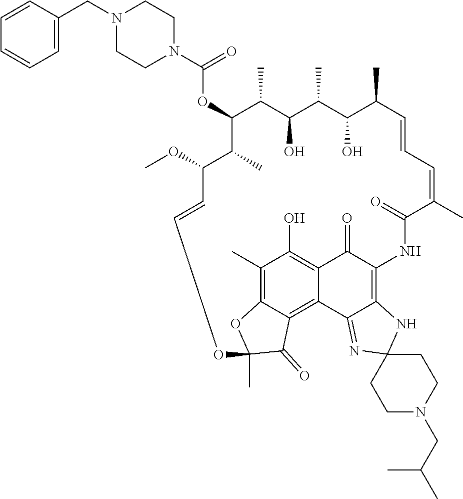 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccccc3)CC2)[C@@H]1C)C4=O] |
| 20 | 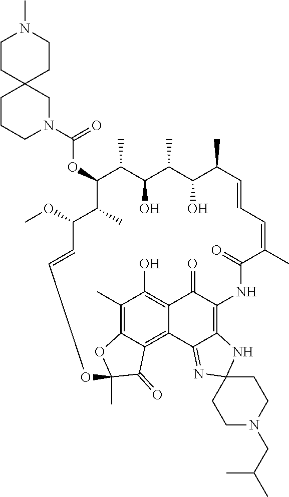 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CCN(C)CC3)C2)[C@@H]1C)C4=O] |
| 21 | 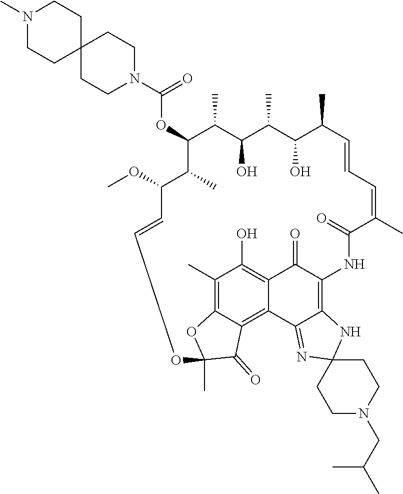 |
| 24 | 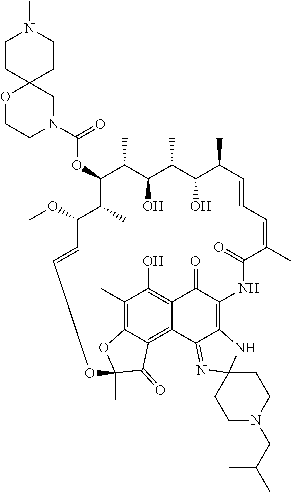 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCOC3(CCN(C)CC3)C2)[C@@H]1C)C4=O] |
| 25 | 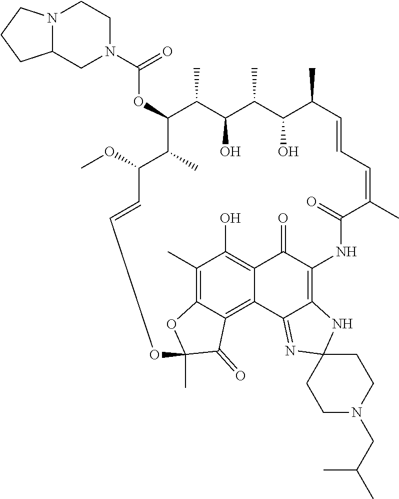 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN3CCCC3C2)[C@@H]1C)C4=O] |
| 29 | 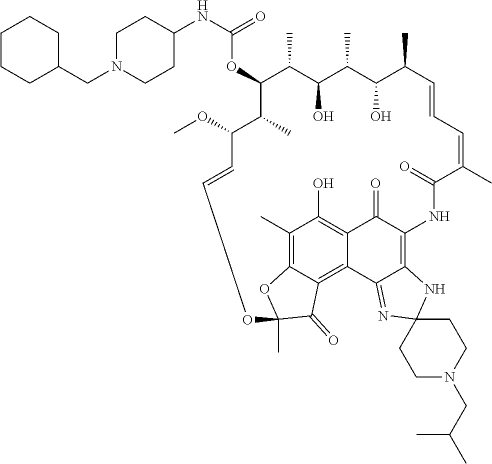 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(CC3CCCCC3)CC2)[C@@H]1C)C4=O] |
| 30 | 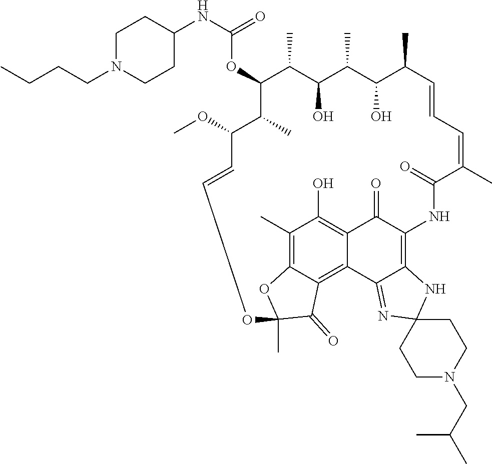 [SMILES: CCCCN1CCC(NC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1] |
| 32, 32a | 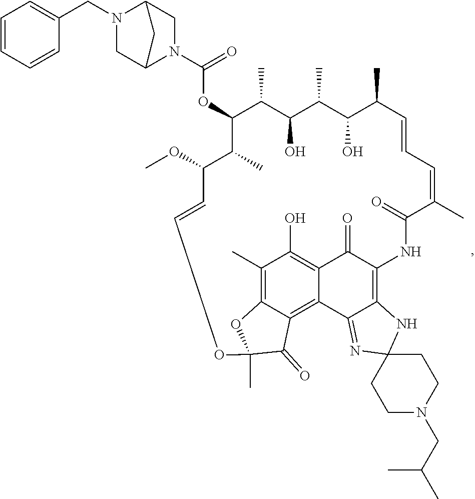 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3CC2CN3Cc2ccccc2)[C@@H]1C)C4=O] preferably 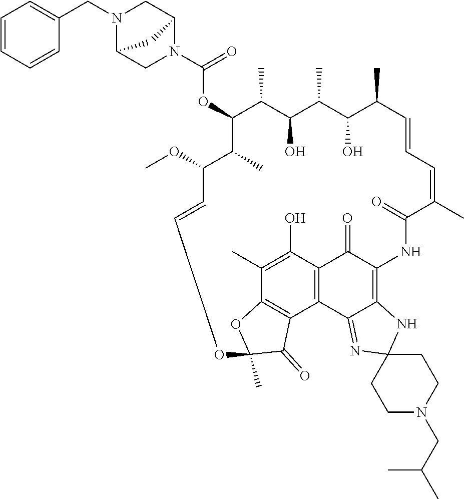 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@@H]3C[C@H]2CN3Cc2ccccc2)[C@@H]1C)C4=O] |
| 36 | 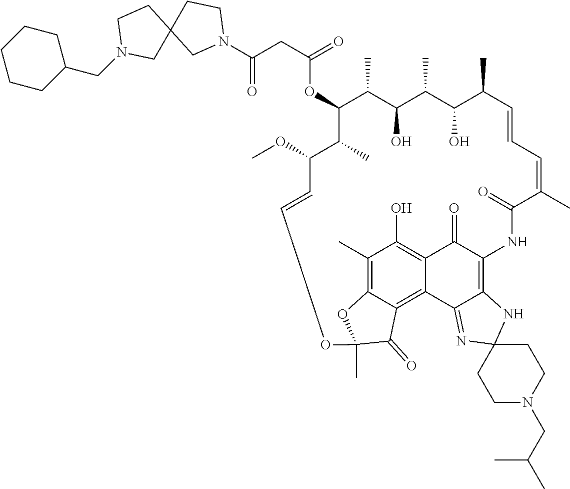 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CC(=O)N2CCC3(CCN(CC5CCCCC5)C3)C2)[C@@H]1C)C4=O] |
| 37 | 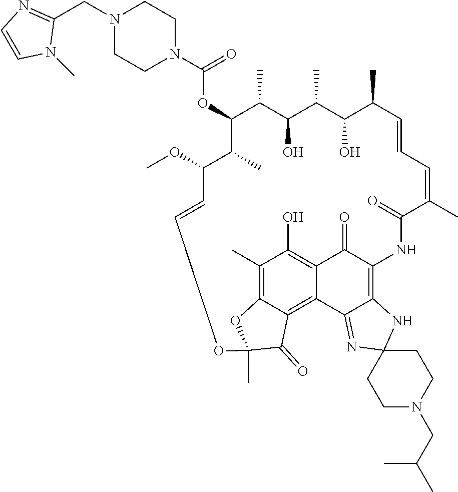 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3nccn3C)CC2)[C@@H]1C)C4=O] |
| 38 | 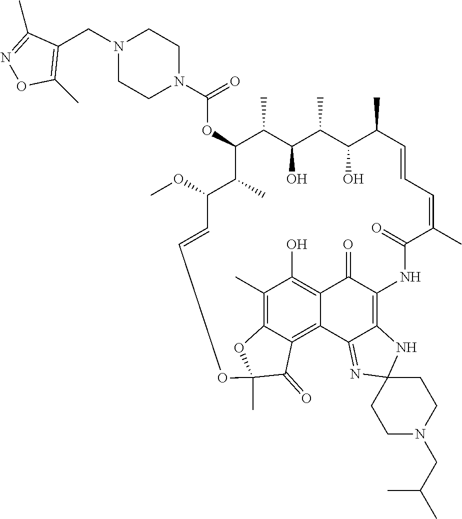 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3c(C)noc3C)CC2)[C@@H]1C)C4=O] |
| 41 | 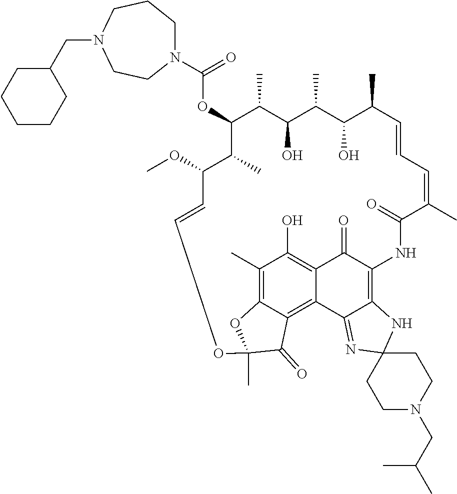 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(CC3CCCCC3)CC2)[C@@H]1C)C4=O] |
| 46 | 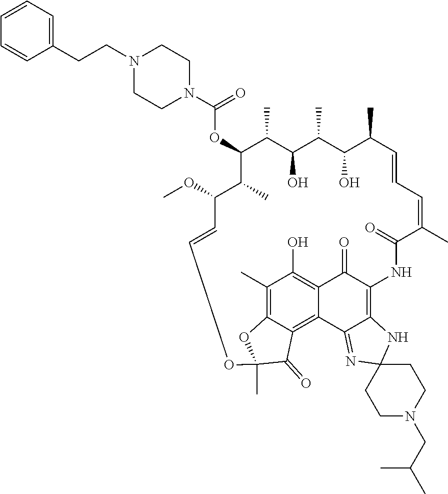 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCc3ccccc3)CC2)[C@@H]1C)C4=O] |
| 48 | 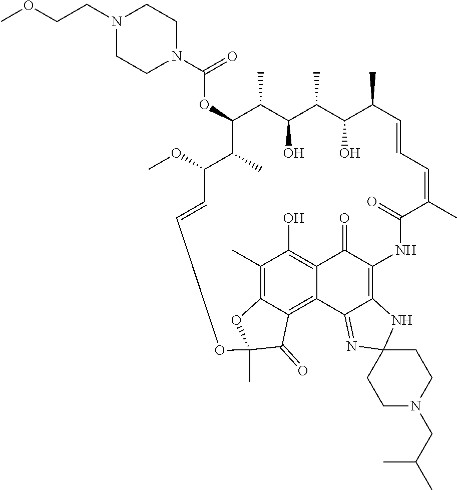 |
| 49 | 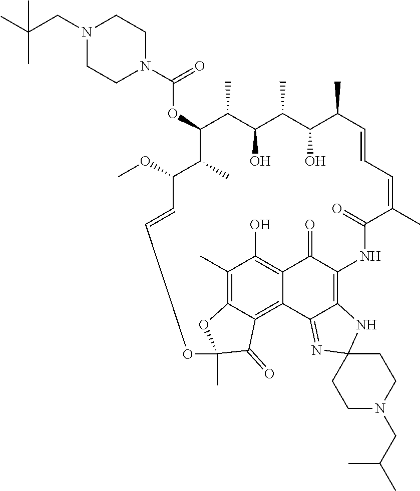 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC(C)(C)C)CC2)[C@@H]1C)C4=O] |
| 50 | 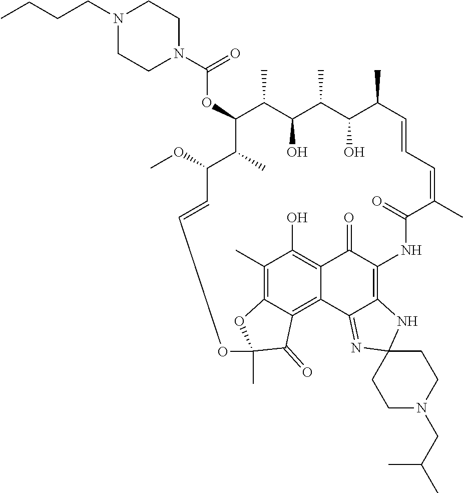 [SMILES: CCCCN1CCN(C(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1] |
| 52 | 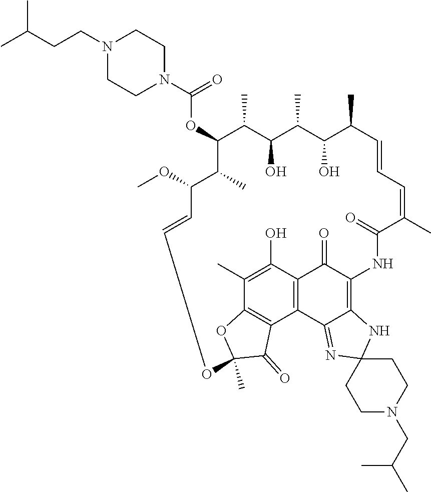 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCC(C)C)CC2)[C@@H]1C)C4=O] |
| 53 | 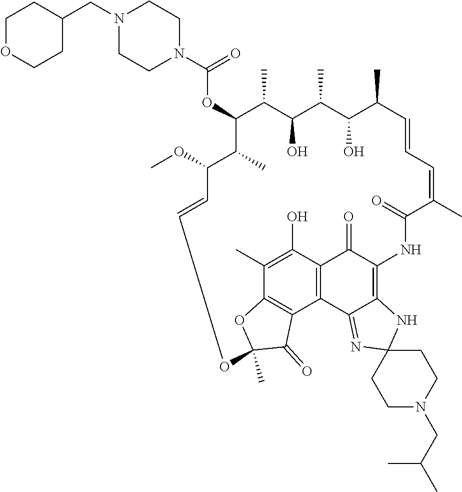 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCOCC3)CC2)[C@@H]1C)C4=O] |
| 54 | 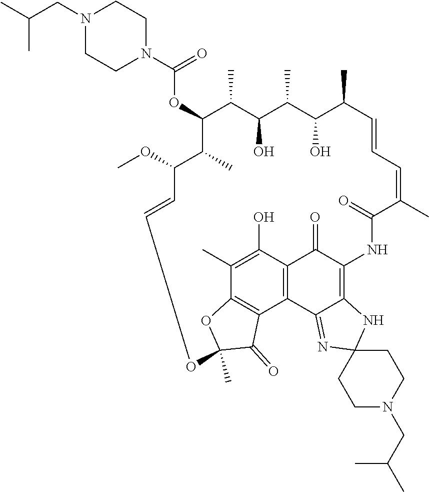 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC(C)C)CC2)[C@@H]1C)C4=O] |
| 55 | 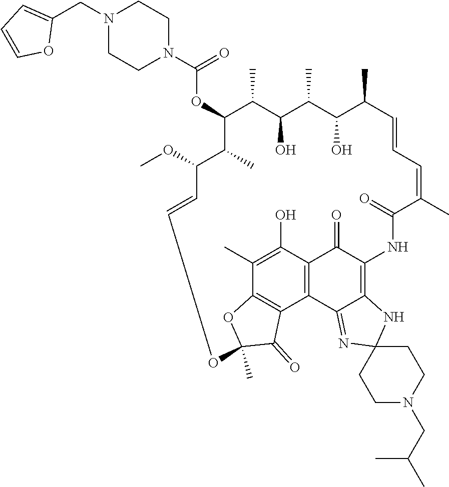 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccco3)CC2)[C@@H]1C)C4=O] |
| 56 | 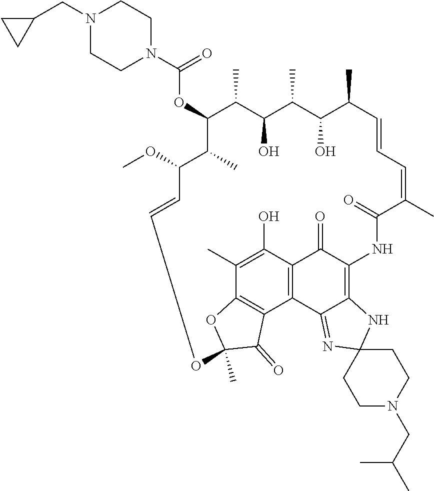 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CC3)CC2)[C@@H]1C)C4=O] |
| 57 | 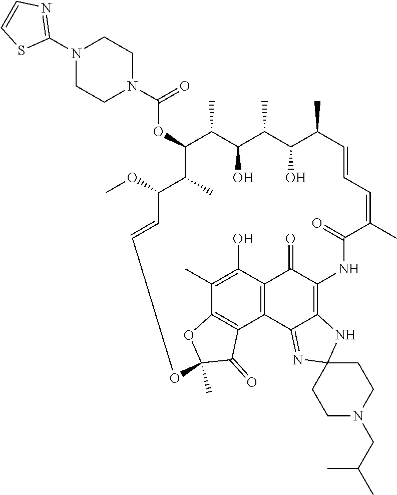 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3nccs3)CC2)[C@@H]1C)C4=O] |
| 58 | 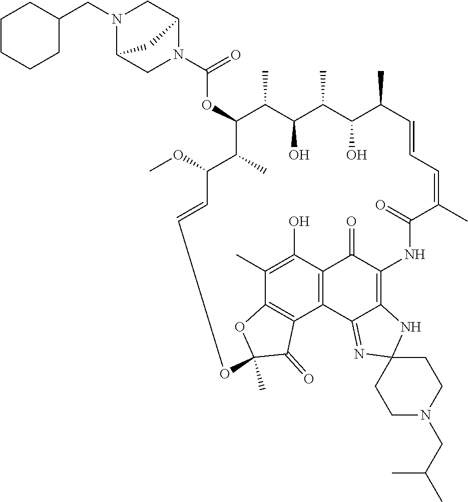 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@@H]3C[C@H]2CN3CC2CCCCC2)[C@@H]1C)C4=O] |
| 59 | 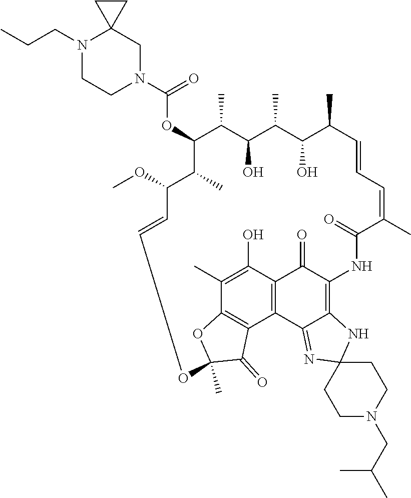 [SMILES: CCCN1CCN(C(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC12CC2] |
| 60 | 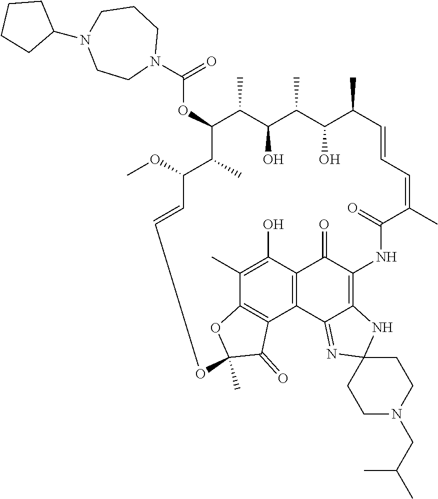 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(C3CCCC3)CC2)[C@@H]1C)C4=O] |
| 62 | 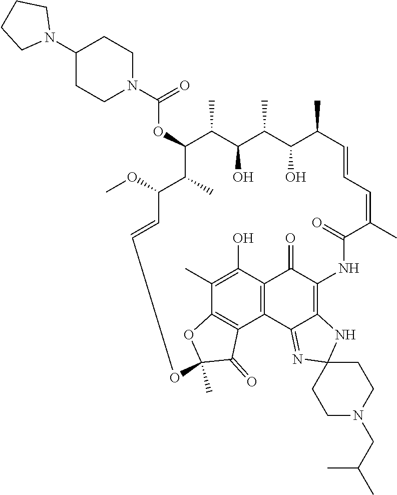 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(N3CCCC3)CC2)[C@@H]1C)C4=O] |
| 63 | 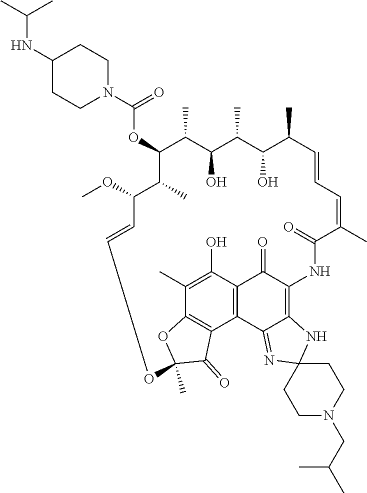 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(NC(C)C)CC2)[C@@H]1C)C4=O] |
| 65 | 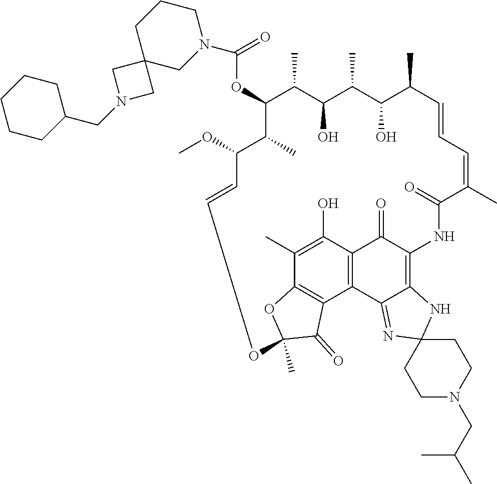 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CN(CC5CCCCC5)C3)C2)[C@@H]1C)C4=O] |
| 66 | 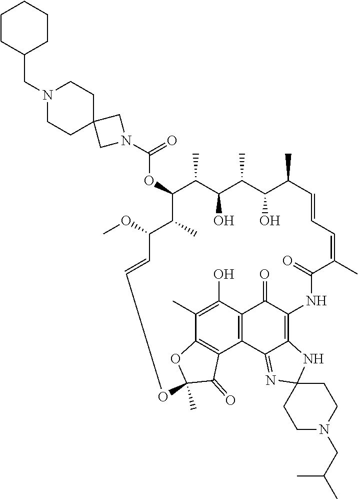 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3(CCN(CC5CCCCC5)CC3)C2)[C@@H]1C)C4=O] |
| 67 | 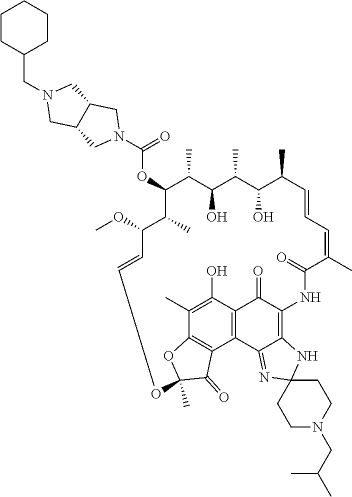 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@H]3CN(CC5CCCCC5)C[C@H]3C2)[C@@H]1C)C4=O] |
| 68 | 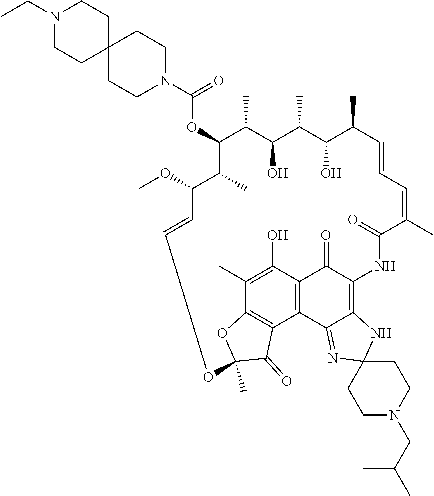 [SMILES: CCN1CCC2(CC1)CCN(C(=O)O[C@H]1[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]1C)O5)C3=O)CC2] |
| 69 | 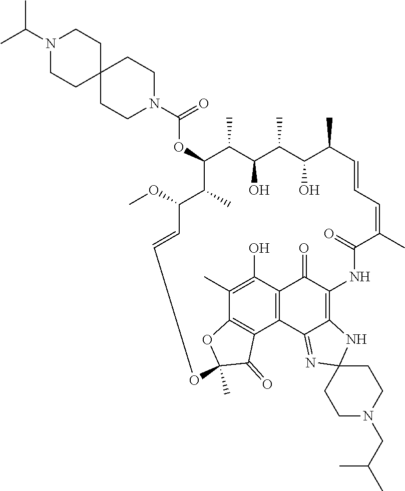 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CC2)CCN(C(C)C)CC3)[C@@H]1C)C4=O] |
| 70 | 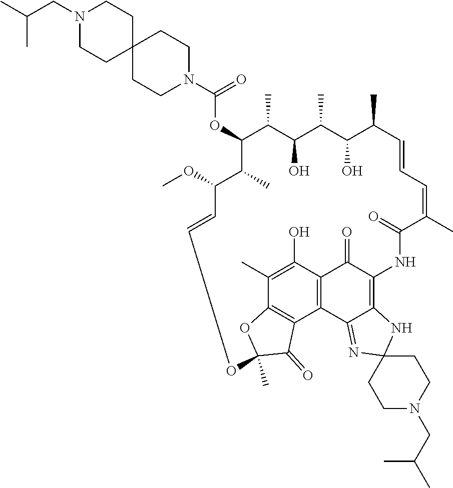 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC(C)C)CC3)CC2)[C@@H]1C)C4=O] |
| 71 | 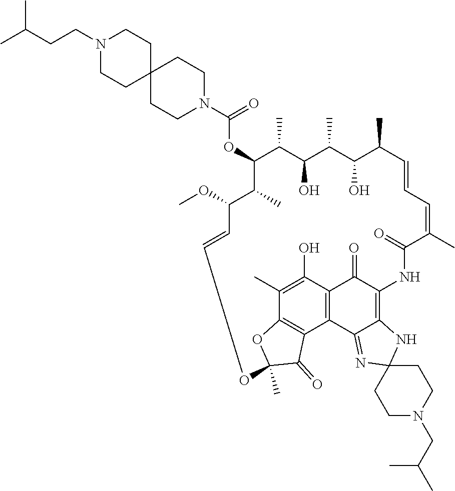 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CCC(C)C)CC3)CC2)[C@@H]1C)C4=O] |
| 72 | 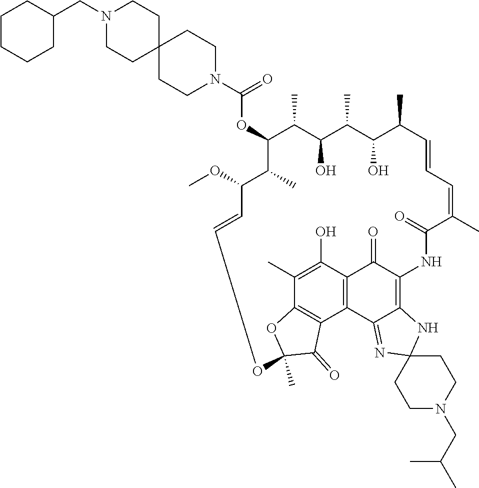 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC5CCCCC5)CC3)CC2)[C@@H]1C)C4=O] |
| 73 | 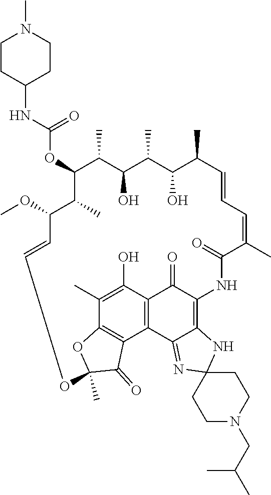 |
| 74 | 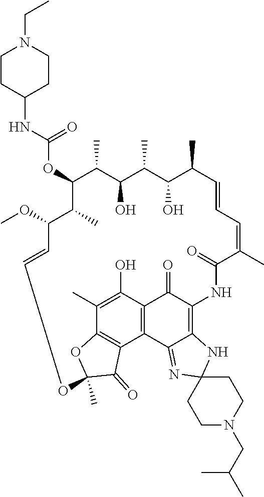 |
| 75 | 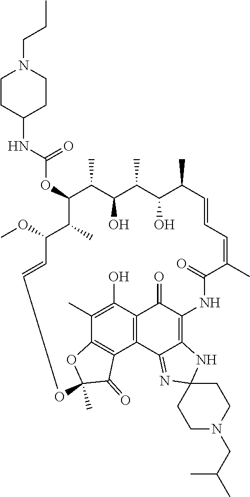 |
| 76 | 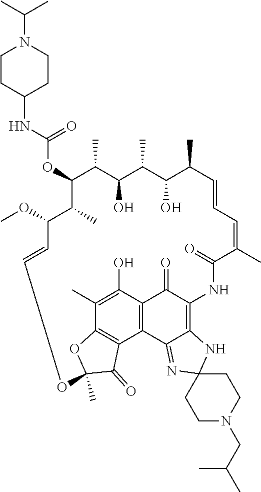 |
| 77 | 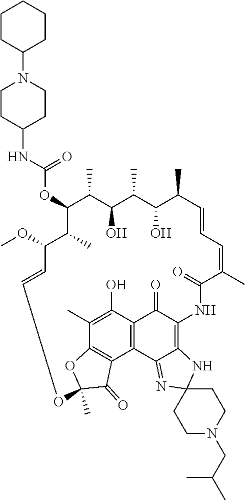 |
| 78 | 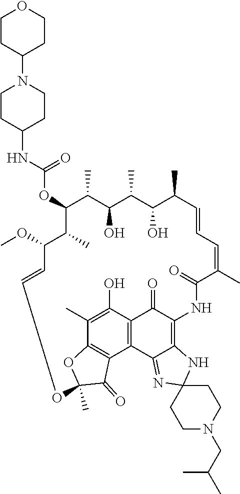 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(C3CCOCC3)CC2)[C@@H]1C)C4=O] |
| 83 | 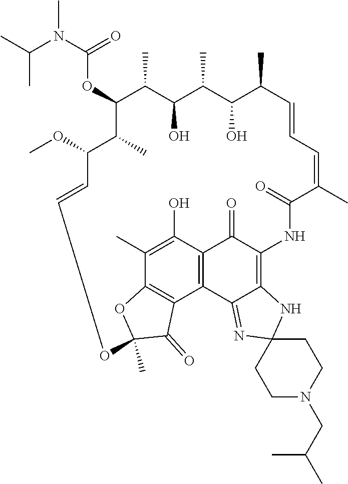 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)C(C)C)[C@@H]1C)C4=O] |

In some embodiments, the compound is selected from the group consisting of Compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, or a pharmaceutically acceptable salt thereof.

In some embodiments, the compound is selected from the group consisting of Compound 1, 2, 3, 4, 7, 8, 9, 10, 11, 12, 13, 14, 15, and 17, or a pharmaceutically acceptable salt thereof.

In some embodiments, the compound is selected from the group consisting of Compound 1, 2, 3, 4, 7, 8, 9, 10, 14, and 17, or a pharmaceutically acceptable salt thereof.

In some embodiments, the compound is selected from the group consisting of Compound 1, 2, 3, 4, 7, 8, 9, 10, 11, 12, 13, 14, 15, 17, 20, 21, 24, 25, 29, 30, 32a, 36, 37, 38, 41, 43, 44, 46, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 82, and 83, or a pharmaceutically acceptable salt thereof.

In some embodiments, the compound is selected from the group consisting of Compound 1, 2, 4, 7, 8, 9, 10, 14, 17, 29, 36, 41, 49, 50, 52, 53, 54, 56, 58, 59, 60, 65, 66, 67, 70, 71, and 72, or a pharmaceutically acceptable salt thereof.

In some embodiments, the compound is selected from the group consisting of Compound 1, 2, 4, 7, 8, 9, 10, 14, and 17, or a pharmaceutically acceptable salt thereof

In some embodiments, the compound of Formula I is Compound 1, or a pharmaceutically acceptable salt thereof. In some embodiments, the compound of Formula I is Compound 2, or a pharmaceutically acceptable salt thereof. In some embodiments, the compound of Formula I is Compound 3, or a pharmaceutically acceptable salt thereof. In some embodiments, the compound of Formula I is Compound 4, or a pharmaceutically acceptable salt thereof. In some embodiments, the compound of Formula I is Compound 5, or a pharmaceutically acceptable salt thereof. In some embodiments, the compound of Formula I is Compound 6, or a pharmaceutically acceptable salt thereof. In some embodiments, the compound of Formula I is Compound 7, or a pharmaceutically acceptable salt thereof. In some embodiments, the compound of Formula I is Compound 8, or a pharmaceutically acceptable salt thereof. In some embodiments, the compound of Formula I is Compound 9, or a pharmaceutically acceptable salt thereof. In some embodiments, the compound of Formula I is Compound 10, or a pharmaceutically acceptable salt thereof. In some embodiments, the compound of Formula I is Compound 11, or a pharmaceutically acceptable salt thereof. In some embodiments, the compound of Formula I is Compound 12, or a pharmaceutically acceptable salt thereof. In some embodiments, the compound of Formula I is Compound 13, or a pharmaceutically acceptable salt thereof. In some embodiments, the compound of Formula I is Compound 14, or a pharmaceutically acceptable salt thereof. In some embodiments, the compound of Formula I is Compound 15, or a pharmaceutically acceptable salt thereof. In some embodiments, the compound of Formula I is Compound 16, or a pharmaceutically acceptable salt thereof. In some embodiments, the compound of Formula I is Compound 17, or a pharmaceutically acceptable salt thereof. In some embodiments, the compound of Formula I is Compound 18, or a pharmaceutically acceptable salt thereof. In some embodiments, the compound of Formula I is Compound 19, or a pharmaceutically acceptable salt thereof. In some embodiments, the compound of Formula I is Compound 20, or a pharmaceutically acceptable salt thereof. In some embodiments, the compound of Formula I is Compound 21, or a pharmaceutically acceptable salt thereof. In some embodiments, the compound of Formula I is Compound 22, or a pharmaceutically acceptable salt thereof. In some embodiments, the compound of Formula I is Compound 23, or a pharmaceutically acceptable salt thereof. In some embodiments, the compound of Formula I is Compound 24, or a pharmaceutically acceptable salt thereof. In some embodiments, the compound of Formula I is Compound 25, or a pharmaceutically acceptable salt thereof. In some embodiments, the compound of Formula I is Compound 26, or a pharmaceutically acceptable salt thereof. In some embodiments, the compound of Formula I is Compound 27, or a pharmaceutically acceptable salt thereof. In some embodiments, the compound of Formula I is Compound 28, or a pharmaceutically acceptable salt thereof. In some embodiments, the compound of Formula I is Compound 29, or a pharmaceutically acceptable salt thereof. In some embodiments, the compound of Formula I is Compound 30, or a pharmaceutically acceptable salt thereof. In some embodiments, the compound of Formula I is Compound 31, or a pharmaceutically acceptable salt thereof. In some embodiments, the compound of Formula I is Compound 32, or a pharmaceutically acceptable salt thereof. In some embodiments, the compound of Formula I is Compound 33, or a pharmaceutically acceptable salt thereof. In some embodiments, the compound of Formula I is Compound 34, or a pharmaceutically acceptable salt thereof. In some embodiments, the compound of Formula I is Compound 35, or a pharmaceutically acceptable salt thereof. In some embodiments, the compound of Formula I is Compound 36, or a pharmaceutically acceptable salt thereof. In some embodiments, the compound of Formula I is Compound 37, or a pharmaceutically acceptable salt thereof. In some embodiments, the compound of Formula I is Compound 38, or a pharmaceutically acceptable salt thereof. In some embodiments, the compound of Formula I is Compound 39, or a pharmaceutically acceptable salt thereof. In some embodiments, the compound of Formula I is Compound 40, or a pharmaceutically acceptable salt thereof. In some embodiments, the compound of Formula I is Compound 41, or a pharmaceutically acceptable salt thereof. In some embodiments, the compound of Formula I is Compound 42, or a pharmaceutically acceptable salt thereof. In some embodiments, the compound of Formula I is Compound 43, or a pharmaceutically acceptable salt thereof. In some embodiments, the compound of Formula I is Compound 44, or a pharmaceutically acceptable salt thereof. In some embodiments, the compound of Formula I is Compound 45, or a pharmaceutically acceptable salt thereof. In some embodiments, the compound of Formula I is Compound 46, or a pharmaceutically acceptable salt thereof. In some embodiments, the compound of Formula I is Compound 47, or a pharmaceutically acceptable salt thereof. In some embodiments, the compound of Formula I is Compound 48, or a pharmaceutically acceptable salt thereof. In some embodiments, the compound of Formula I is Compound 49, or a pharmaceutically acceptable salt thereof. In some embodiments, the compound of Formula I is Compound 50, or a pharmaceutically acceptable salt thereof. In some embodiments, the compound of Formula I is Compound 51, or a pharmaceutically acceptable salt thereof. In some embodiments, the compound of Formula I is Compound 52, or a pharmaceutically acceptable salt thereof. In some embodiments, the compound of Formula I is Compound 53, or a pharmaceutically acceptable salt thereof. In some embodiments, the compound of Formula I is Compound 54, or a pharmaceutically acceptable salt thereof. In some embodiments, the compound of Formula I is Compound 55, or a pharmaceutically acceptable salt thereof. In some embodiments, the compound of Formula I is Compound 56, or a pharmaceutically acceptable salt thereof. In some embodiments, the compound of Formula I is Compound 57, or a pharmaceutically acceptable salt thereof. In some embodiments, the compound of Formula I is Compound 58, or a pharmaceutically acceptable salt thereof. In some embodiments, the compound of Formula I is Compound 59, or a pharmaceutically acceptable salt thereof. In some embodiments, the compound of Formula I is Compound 60, or a pharmaceutically acceptable salt thereof. In some embodiments, the compound of Formula I is Compound 61, or a pharmaceutically acceptable salt thereof. In some embodiments, the compound of Formula I is Compound 62, or a pharmaceutically acceptable salt thereof. In some embodiments, the compound of Formula I is Compound 63, or a pharmaceutically acceptable salt thereof. In some embodiments, the compound of Formula I is Compound 64, or a pharmaceutically acceptable salt thereof. In some embodiments, the compound of Formula I is Compound 65, or a pharmaceutically acceptable salt thereof. In some embodiments, the compound of Formula I is Compound 66, or a pharmaceutically acceptable salt thereof. In some embodiments, the compound of Formula I is Compound 67, or a pharmaceutically acceptable salt thereof. In some embodiments, the compound of Formula I is Compound 68, or a pharmaceutically acceptable salt thereof. In some embodiments, the compound of Formula I is Compound 69, or a pharmaceutically acceptable salt thereof. In some embodiments, the compound of Formula I is Compound 70, or a pharmaceutically acceptable salt thereof. In some embodiments, the compound of Formula I is Compound 71, or a pharmaceutically acceptable salt thereof. In some embodiments, the compound of Formula I is Compound 72, or a pharmaceutically acceptable salt thereof. In some embodiments, the compound of Formula I is Compound 73, or a pharmaceutically acceptable salt thereof. In some embodiments, the compound of Formula I is Compound 74, or a pharmaceutically acceptable salt thereof. In some embodiments, the compound of Formula I is Compound 75, or a pharmaceutically acceptable salt thereof. In some embodiments, the compound of Formula I is Compound 76, or a pharmaceutically acceptable salt thereof. In some embodiments, the compound of Formula I is Compound 77, or a pharmaceutically acceptable salt thereof. In some embodiments, the compound of Formula I is Compound 78, or a pharmaceutically acceptable salt thereof. In some embodiments, the compound of Formula I is Compound 79, or a pharmaceutically acceptable salt thereof. In some embodiments, the compound of Formula I is Compound 80, or a pharmaceutically acceptable salt thereof. In some embodiments, the compound of Formula I is Compound 81, or a pharmaceutically acceptable salt thereof. In some embodiments, the compound of Formula I is Compound 82, or a pharmaceutically acceptable salt thereof. In some embodiments, the compound of Formula I is Compound 83, or a pharmaceutically acceptable salt thereof. In some embodiments, the compound of Formula I is Compound 84, or a pharmaceutically acceptable salt thereof.

In one aspect, the present invention provides a pharmaceutical composition comprising at least one compound according to the present invention, or a pharmaceutically acceptable salt, tautomer, solvate or hydrate thereof, and a pharmaceutically acceptable excipient.

### Methods of Synthesizing the Disclosed Compounds

The compounds of the present invention may be made by a variety of methods, including standard chemistry. The methods include but are not limited to the methods described in the suitable synthetic routes depicted in the schemes given below.

The compounds of the present invention may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of the present invention.

Those skilled in the art will recognize if a stereocenter exists in the compounds of Formula I. Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley interscience, 1994).

The compounds described herein may be made from commercially available starting materials or synthesized using known organic, inorganic, and/or enzymatic processes.

### Preparation of Compounds

Compounds of the present invention can be synthesized by following the steps outlined in General Schemes 1, 2 and 3. Starting materials are either commercially available or made by known procedures in the reported literature or as illustrated.

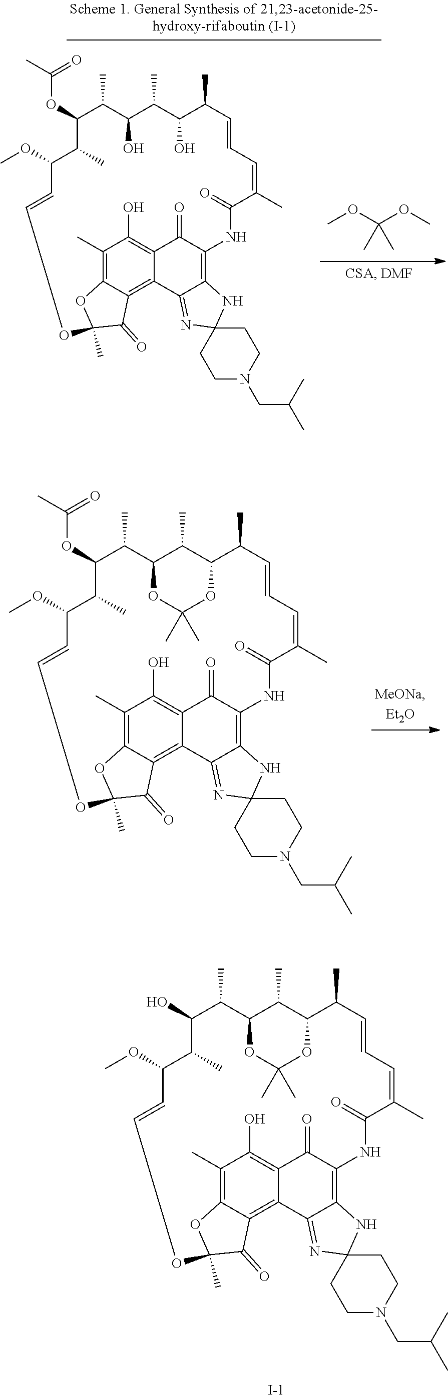

A general method for the preparation of 21,23-acetonide-25-hydroxy-rifabutin I-1 is shown above in Scheme 1. Protection of the C21 and C23 hydroxy groups using a suitable protecting group such as dimethoxy propane and camphorsulphonic acid in a solvent, e.g., DMF afforded an acetal-protected rifabutin. De-acetylation of the protected rifabutin using a basic solution such as sodium methoxide in ether afforded the C25-hydroxy, C21-C23-protected rifabutin (I-1).

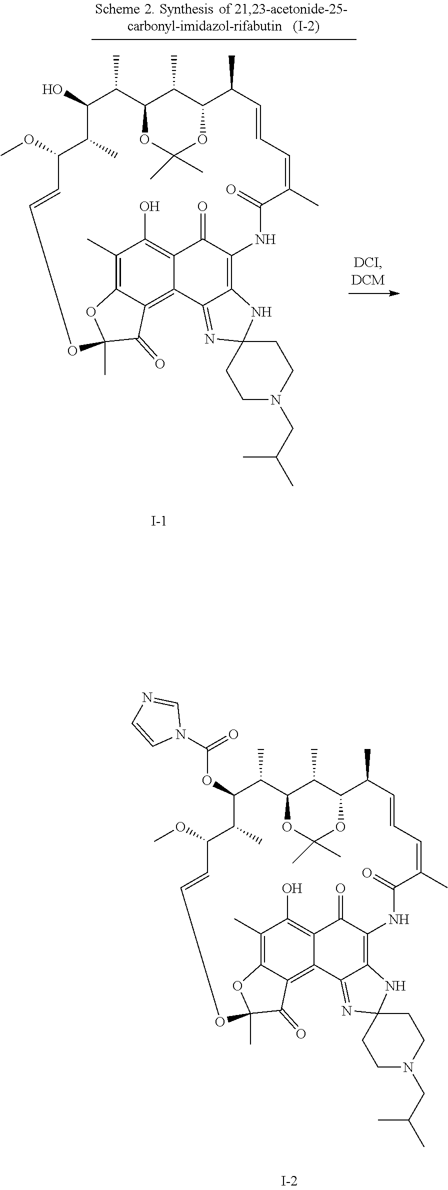

The free C25-hydroxy group of Intermediate (I-1) can be converted to a C₂₅ imidazole carbamate (i.e., Intermediate I-2) using a carbamoylation reagent such as 1,1′-carbonyldiimidazole in a suitable solvent such as dichloromethane (DCM) as shown above in Scheme 2.

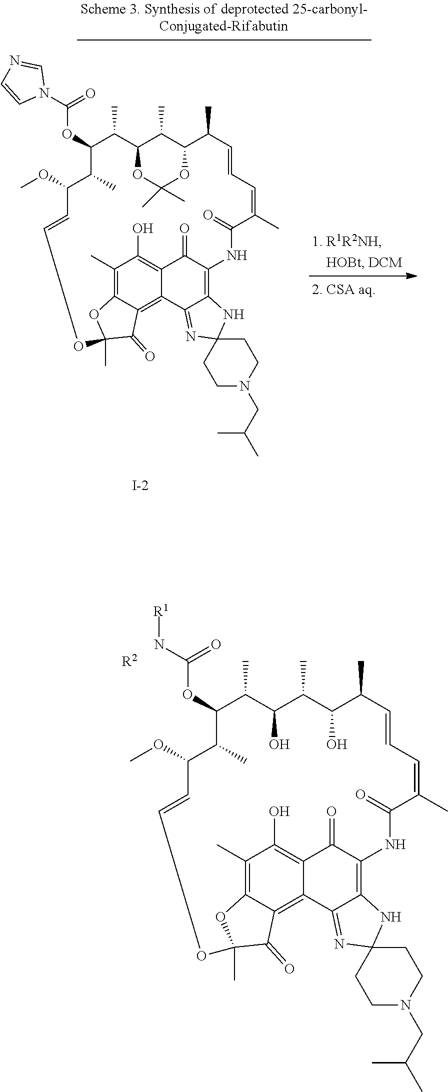

Intermediate (1-2) can be substituted by an appropriate amine in the presence of a suitable coupling reagent such as 1-hydroxy-1H-benzotriazole (HOBt). Subsequent deprotection of the C21-C23 acetal in the conjugated rifabutin can be carried out by treating with an acid such as camphorsulfonic acid in a suitable solvent such as a mixture of THF/water.

### Antibacterial Efficacy of the Disclosed Compounds

The inventive compounds are C25-modified analogs of rifabutin that exhibit broad spectrum antibacterial activity characteristic of the rifamycin class. Additionally, the inventive compounds unexpectedly showed enhanced antibacterial activity against non-tuberculous Mycobacteria including M. abscessus compared to currently available antibiotics (e.g., rifabutin).

As shown below in Example 4, Table 6, the compounds of the invention are effective at inhibiting bacterial growth in strains of S. aureus, A. baumannii, M. abscessus, M. kansasii, M. xenopi, and M. avium.

Rifabutin exhibited modest activity against the tested M. abscessus strains with an MIC value of 8 mg/L in 7H9 broth and 16 mg/L in Mueller Hinton broth. In contrast, Compounds of the invention showed considerably lower MIC values, corresponding to up to 132-fold increased activity over rifabutin. Accordingly, the present invention teaches compounds that display increased activity against M. abscessus beyond that of antibiotics known in the literature. In some embodiments, the inventive compounds can be used to treat M. abscessus infection. In some embodiments, the inventive compounds can be used to treat M. abscessus infection that is resistant to current antibiotics.

Accordingly, in some embodiments, the inventive compounds can be used to inhibit bacterial infection, preferably infection caused by a non-tuberculous Mycobacteria, more preferably M. abscessus. In some embodiments, the inventive compounds can be used to inhibit bacterial infection caused by A. baumannii.

### Methods of Using the Disclosed Compounds

An aspect of the present invention relates to a compound of Formula I or a pharmaceutically acceptable salt thereof for use as a medicament.

In one aspect, the present invention provides a compound according to Formula I of the present invention or a pharmaceutically acceptable salt, tautomer, solvate or hydrate thereof, or a pharmaceutical composition comprising a compound of Formula I of the present invention, for use in a method of preventing or treating a disease in a subject, preferably an infection, further preferably a bacterial infection.

In one aspect, the present invention provides a method of treating a disease, preferably an infection, more preferably a bacterial infection in a subject in need thereof, the method comprising administering to the subject an effective amount of a compound of Formula I of the present invention or a pharmaceutically acceptable salt, tautomer, solvate, or hydrate thereof. In one aspect, the present invention provides a method of treating a disease, preferably an infection, more preferably a bacterial infection in a subject in need thereof, the method comprising administering to the subject an effective amount of a pharmaceutical composition comprising a compound of Formula I of the present invention or a pharmaceutically acceptable salt, tautomer, solvate, or hydrate thereof.

In one aspect, the present invention provides the use of a compound of Formula I of the present invention or a pharmaceutically acceptable salt, tautomer, solvate, or hydrate thereof in the manufacture of a medicament for treating a disease, preferably an infection, more preferably a bacterial infection in a subject in need thereof. In one aspect, the present invention provides the use of a pharmaceutical composition comprising a compound of Formula I of the present invention or a pharmaceutically acceptable salt, tautomer, solvate, or hydrate thereof in the manufacture of a medicament for treating a disease, preferably an infection, more preferably a bacterial infection in a subject in need thereof.

In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In some preferred embodiments, the infection is caused by one or more bacterium belonging to a genus of non-tuberculosis Mycobacterium, preferably M. abscessus, M. avium, M kansasii, M. smegmatis, M. xenopi and/or M. malmoense, more preferably M. abscessus, M avium, M. kansasii, and/or M. xenopi, yet more preferably M. abscessus.

In some embodiments, the infection is caused by one or more bacterium belonging to the species M. abscessus, A. baumannii, and/or S. aureus. In some embodiments, the infection is caused by one or more bacterium belonging to the species M. abscessus. In some embodiments, the infection is caused by one or more bacterium belonging to the species A. baumannii.

In some preferred embodiments, the inventive compounds are used for the treatment of a non-tuberculous Mycobacteria that causes a non-tuberculous Mycobacteria pulmonary infection. In some embodiments the non-tuberculosis Mycobacterium is M. abscessus, M. avium, M. kansasii, M. smegmatis, M. xenopi and/or M. malmoense, preferably M. abscessus, M avium, M. kansasii, and/or M. xenopi, more preferably M. abscessus. Accordingly, in some preferred embodiments, the inventive compounds are for use in the treatment of a non-tuberculous Mycobacteria pulmonary infection. In some preferred embodiments, the inventive compounds are for use in the manufacture of a medicament for the treatment of a non-tuberculous Mycobacteria pulmonary infection. In some preferred embodiments, the present invention provides a method of treating a non-tuberculous Mycobacteria pulmonary infection in a subject in need thereof, comprising administering to the subject an effective amount of an inventive compound of the invention.

Illustrative pharmaceutical compositions are tablets and gelatin capsules comprising a compound of the invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

The inventive compounds and pharmaceutical compositions may be administered by any suitable route, e.g. orally, for example as a syrup, tablet, capsule, controlled-release preparation, fast-dissolving preparation, or lozenge.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions, using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a compound of the present invention and a pharmaceutically acceptable carrier. The pharmaceutically acceptable carrier can further include an excipient, diluent, or surfactant. Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter, or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EQUIVALENTS

While the present technology has been described in conjunction with the specific embodiments set forth above, many alternatives, modifications and other variations thereof will be apparent to those of ordinary skill in the art. All such alternatives, modifications and variations are intended to fall within the spirit and scope of the present invention.

### EXAMPLES

The invention will now be illustrated by way of the following non-limiting examples. While particular embodiments of the invention are described below, a skilled person will appreciate that various changes and modifications can be made. References to preparations carried out in a similar manner to, or by the general method of, other preparations, may encompass variations in routine parameters such as time, temperature, workup conditions, minor changes in reagents amounts, and the like.

### Abbreviations

The following list provides definitions of certain abbreviations and symbols as used herein. It will be appreciated that the list is not exhaustive, but the meaning of those abbreviations and symbols not herein below defined will be readily apparent to those skilled in the art. In describing the invention, chemical elements are identified in accordance with the Periodic Table of the Elements.
ACN acetonitrile
AcOH acetic acid
aq. aqueous
Ar argon
CSA camphorsulphonic acid
DCM dichloromethane
DMF dimethylformamide
eq. equivalent
EtOAc ethyl acetate
EtOH ethanol
h hour(s)
HOBt 1-hydroxy-TH-benzotriazole
HPLC high performance liquid chromatography
LC liquid chromatography
M molar
MeOH methanol
MS mass spectroscopy
min minutes
NMR nuclear magnetic resonance spectroscopy
rt room temperature
Rt retention time
sat. saturated

Unless specified otherwise, the purity and identity of Intermediate or example compounds were assessed by state-of-the-art HLPC-MS. Methods are described below.

### Characterization of the Compounds

Method A was used for the intermediates. Method A or B was used to measure final purity of the compounds as noted herein. Purity (%) was determined by reversed phase HPLC or UPLC, using UV detection (254 nm). Structure was confirmed by MS, using electro spray ionization positive (ESI+) method and reported as [M+H]⁺, referring to the protonated molecular ion.

### Method A:

UPLC system: UPLC I BIN SOL MGR with ACQUITY UPLC I-Class eK PDA Detector; Column: Acquity BEH C₁₈ column (1.7 μm particle size, dimensions 50 mm×2.1 mm); Mobile phases: phase A (H₂O/ammonium formate, pH 3.75 (A) or 9.2 (B)) and phase B (CH₃CN+5% H₂O/ammonium formate, pH 3.75 (A) or 9.2 (B)) were used according to the following methods:

Method A

| Time | | | Flow |
|---|---|---|---|
| (min) | Phase A | Phase B | (mL/min) |
| 0.00 | 98.0 | 2.0 | 0.6 |
| 0.20 | 98.0 | 2.0 | 0.6 |
| 2.50 | 0.0 | 100.0 | 0.6 |
| 3.50 | 0.0 | 100.0 | 0.6 |
| 3.60 | 98.0 | 2.0 | 0.6 |
| 5.00 | 98.0 | 2.0 | 0.6 |

Mass spectrometer: ACQUITY QDa (Performance) Xevo TQD. Ionization: electrospray (polarity: negative and positive). Unless otherwise stated, analyses lasted five minutes. In some cases, analyses were performed for 10 min (*) or 30 min (**) as indicated.

### Method B:

HPLC system: Waters 2695 LC with photodiode array detector Waters 996; Column: XBridge C₁₈ column (3.5 μm particle size, dimensions 50 mm×4.6 mm); Mobile phases: phase A (H₂O/ammonium formate, pH 9.2) and phase B (CH₃CN+5% H₂O/ammonium formate, pH 9.2) were used according to the following methods:

Method B

| Time | | | Flow |
|---|---|---|---|
| (min) | Phase A | Phase B | (mL/min) |
| 0.00 | 100.0 | 0.0 | 2.00 |
| 1.00 | 100.0 | 0.0 | 2.00 |
| 25.00 | 0.0 | 100.0 | 2.00 |
| 27.00 | 0.0 | 100.0 | 2.00 |
| 27.10 | 100.0 | 0.0 | 2.00 |
| 30.00 | 100.0 | 0.0 | 2.00 |

Mass spectrometer: Waters Alliance Micromass ZQ 2000. Ionization: electrospray (polarity: negative and positive).

### NMR Analysis

NMR spectra were recorded on a Bruker DRX-300 spectrometer or Bruker 500 MHz spectrometer with a TXI probe. Chemical shifts are in parts per million (ppm). The assignments were made using one-dimensional (1D) ¹H and ¹³C spectra and two-dimensional (2D) HSQC, HMBC spectra.

The compounds of the current invention were obtained from the free 25-OH rifabutin (Intermediate I-1) prepared from commercially available rifabutin as shown below.

### Example 1: Synthesis of 21,23-acetonide-25-hydroxy-rifabutin (I-1)

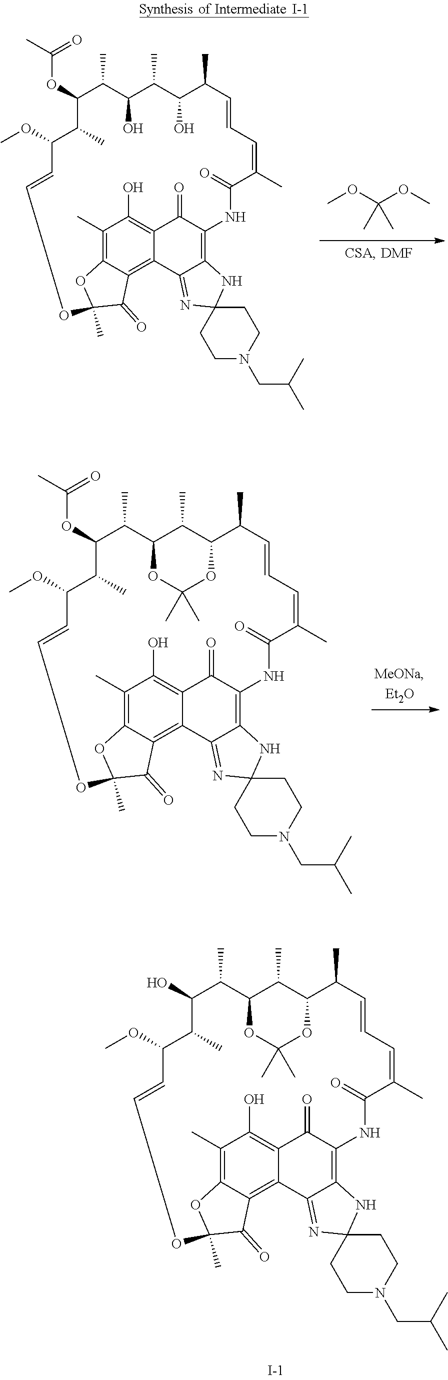

Commercially available rifabutin (40.0 g, 47.2 mmol) was dissolved in dry DMF (80 mL) at rt under nitrogen atmosphere. 2,2-Dimethoxy-propane (58.1 mL, 472 mmol) and camphorsulphonic acid (12.6 g, 54.3 mmol) were sequentially added to the solution. The reaction mixture was stirred at rt for 26 h under nitrogen atmosphere. The mixture was then cooled to 0° C. and poured into a mixture of sat. aq. solution of NaHCO₃ (700 mL) and water (500 mL). The reaction flask was washed with acetone (100 mL). The resulting suspension was stirred in an ice bath for 10 min and filtered. The cake was rinsed with sat. aq. solution of NaHCO₃ (100 mL) and water (50 mL) and dried under vacuum at 40° C. for 24 h. The crude product was purified by flash chromatography (DCM to 50% of the mixture DCM/MeOH/NH₄OH 90/9/1.5 in DCM) to afford the 21,23-acetonide-rifabutin as a purple solid (37.68 g, 90% yield).

LC/MS A (ESI+): tr=3.20 min, m/z [M+H]⁺=887.48, UV purity: 97% (254 nm).

¹H NMR (300 MHz, CDCl₃): δ 0.39 (d, J=7.0 Hz, 3H), 0.68 (d, J=7.0 Hz, 3H), 0.77-0.88 (m, 9H), 0.89-0.98 (m, 6H), 1.23 (s, 3H), 1.36-1.55 (m, 2H), 1.71-2.18 (m, 6H), 1.77 (s, 3H), 1.96 (s, 3H), 2.04 (s, 3H), 2.22-2.35 (m, 3H), 2.31 (s, 3H), 2.52-2.72 (m, 2H), 2.79 (s, 3H), 2.91-3.09 (m, 2H), 3.06 (dd, J=10.3, 5.4 Hz, 1H), 3.34 (d, J=6.7 Hz, 1H), 3.58 (dd, J=3.1 Hz, 1H), 4.91 (d, J=7.5 Hz, 1H), 5.08 (dd, J=12.1, 6.7 Hz, 1H), 5.87 (d, J=12.2 Hz, 1H), 6.08 (dd, J=15.5, 6.8 Hz, 1H), 6.16 (dd, J=10.7, 1.4 Hz, 1H), 6.28 (dd, J=15.5, 10.7 Hz, 1H), 7.75 (s, 1H), 8.75 (s, 1H), 14.82 (s, 1H).

¹³C NMR (75 MHz, CDCl₃): δ 7.89, 9.11, 9.99, 12.8, 18.0, 20.2, 20.3, 20.9, 23.5, 25.8, 34.1, 35.4, 36.0, 36.8, 40.8, 41.4, 51.5, 56.2, 66.3, 71.2, 74.6, 76.9, 78.9, 95.0, 100.1, 104.6, 106.1, 108.7, 111.6, 113.7, 115.3, 123.8, 125.5, 131.2, 132.6, 140.8, 140.9, 142.3, 155.2, 168.2, 168.9, 170.5, 172.2, 181.3, 192.7.

21,23-acetonide-rifabutin (10.6 g, 11.9 mmol) was dissolved in dry diethyl ether (500 mL). The solution was cooled to −10° C. with Ar bubbling. After 15 min, a solution of NaOMe (30 mL, 25 w % in MeOH) was slowly added. Precipitation occurred and another portion of diethyl ether (100 mL) was added to homogenize. NaOMe solution was again added (27.4 mL).

The solution was stirred at −5° C. for 10 min before the ice bath was removed. The reaction mixture was stirred at rt for 6 h. Sat. aq. solution of NaHCO₃ (400 mL) was added and the layers were separated. The aqueous layer was extracted with diethyl ether (400 mL) and the combined organic layers were washed with brine (300 mL) and evaporated to afford the desired crude product as a black-purple powder. The product was purified by flash chromatography (DCM to 50% of the mixture DCM/MeOH/NH₄OH 90/9/1.5 in DCM). After evaporation, the product was redissolved in acetone and slowly added into water under vigorous stirring. The solid was collected by filtration and dried at 40° C. to yield 8.54 g of the Intermediate I-1.

LC/MS A (ESI+): tr=2.98 min, m/z [M+H]⁺=845.59, UV purity: 97% (254 nm).

¹H NMR (300 MHz, CDCl₃): δ 0.48 (d, J=7.0 Hz, 3H), 0.70 (d, J=6.8 Hz, 3H), 0.74-0.84 (m, 9H), 0.86-0.96 (m, 6H), 1.04 (s, 3H), 1.23-1.33 (m, 1H), 1.43-1.66 (m, 2H), 1.72 (s, 3H), 1.75-2.11 (m, 5H), 2.00 (s, 3H), 2.18-2.29 (m, 3H), 2.21 (s, 3H), 2.47-2.71 (m, 2H), 2.85-3.09 (m, 2H), 3.05-3.14 (m, 1H), 3.10 (s, 3H), 3.27-3.35 (m, 1H), 3.38-3.51 (m, 2H), 3.55 (dd, J=9.2, 3.3 Hz, 1H), 4.93 (dd, J=12.5, 9.5 Hz, 1H), 5.93 (dd, J=15.5, 6.5 Hz, 1H), 6.05-6.16 (m, 2H), 6.24 (dd, J=15.5, 11.7 Hz, 1H), 7.72 (s, 1H), 8.63 (s, 1H), 14.83 (s, 1H).

¹³C NMR (75 MHz, CDCl₃): δ 7.7, 8.4, 12.9, 17.8, 19.9, 20.0, 20.9, 21.0, 24.2, 25.5, 25.8, 34.6, 35.2, 36.3, 39.7, 41.1, 51.4, 51.7, 56.2, 66.3, 71.0, 71.5, 75.5, 83.0, 95.0, 99.6, 104.9, 105.7, 108.6, 111.7, 111.8, 114.3, 124.1, 125.5, 132.0, 132.4, 140.3, 142.6, 142.7, 155.2, 168.2, 169.1, 171.3, 181.6, 191.2.

### Example 2: Synthesis of 25-carbonyl-imidazol-21,23-acetonide-Rifabutin (1-2)

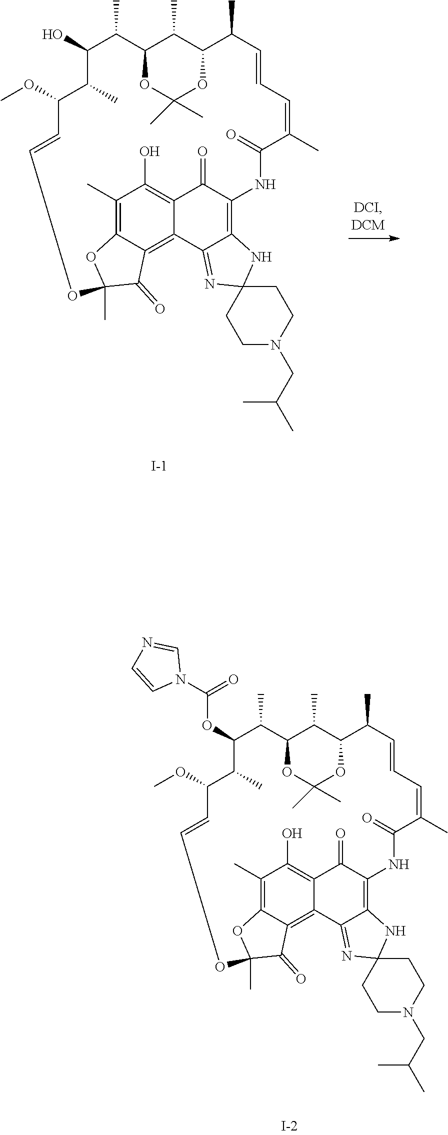

Intermediate I-1 (1.00 g, 1.18 mmol, 1 eq) was dissolved in anhydrous DCM (40 ml) under a nitrogen atmosphere. To this solution was added carbonyldiimidazole (1.92 g, 11.80 mmol, 10eq) and the reaction was stirred at rt. After 16 h, the mixture was washed with an aqueous saturated solution of NaHCO₃ (40 ml) and brine (40 mL). The organic layer was dried over MgSO₄, filtered, and DCM was evaporated under vacuum. The obtained product was purified by flash chromatography (Cyclohexane/Acetone (from 100/0 to 60/40 over 20 CV) to yield 25-carbonyl-imidazol-21,23-acetonide-Rifabutin as a black solid (m=660 mg, yield=59%).

LC/MS A (ESI+): tr=2.29 min, m/z [M+H]⁺=939.52, UV purity: 99% (254 nm).

¹H NMR (300 MHz, CDCl₃): δ 0.45 (d, J=7.0 Hz, 1H), 0.70-0.82 (m, 12H), 0.82-0.90 (m, 6H), 1.08 (s, 3H), 1.38-1.54 (m, 1H), 1.58-2.16 (m, 8H), 1.69 (s, 3H), 1.97 (s, 3H), 2.15-2.27 (m, 3H), 2.20 (s, 3H), 2.44-2.62 (m, 2H), 2.67 (s, 3H), 2.82-3.02 (m, 3H), 3.30 (dd, J=7.4, 1.7 Hz, 1H), 3.51 (dd, J=10.5, 3.0 Hz, 1H), 4.95-5.10 (m, 2H), 5.89 (d, J=12.1 Hz, 1H), 5.97 (dd, J=15.6, 6.9 Hz, 1H), 6.07 (dd, J=10.5, 1.2 Hz, 1H), 6.21 (dd, J=15.6, 10.6 Hz, 1H), 6.91-6.97 (m, 1H), 7.23-7.30 (m, 1H), 7.92-8.03 (m, 2H), 8.46-8.92 (brs, 1H), 14.78 (s, 1H).

¹³C NMR (75 MHz, CDCl₃): δ 7.6, 9.4, 10.2, 12.7, 17.7, 19.9, 20.74, 20.76, 23.16, 25.4, 25.7, 26.7, 34.0, 35.3, 36.1, 36.6, 40.3, 40.6, 51.2, 51.4, 55.7, 66.2, 70.9, 76.0, 78.7, 79.5, 94.9, 99.9, 104.5, 105.7, 108.4, 111.5, 113.0, 113.7, 116.8, 123.8, 125.2, 130.3, 131.5, 132.0, 136.8, 140.0, 141.7, 142.3, 148.1, 154.9, 168.0, 168.7, 171.6, 181.2, 191.9.

### Example 3: Preparation of Compound 4

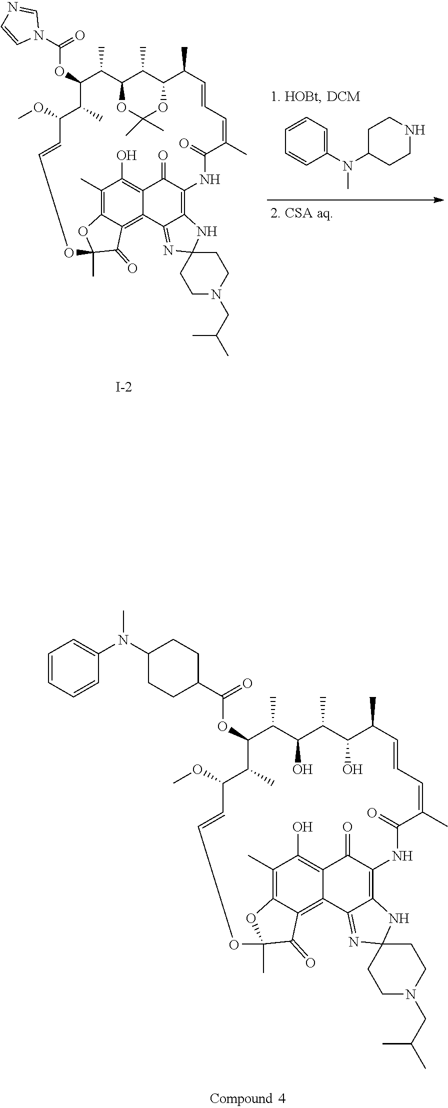

A stock solution of 25-imidazo-carbonyl-21,23-acetonide-rifabutin in DCM (0.025 mol/L) was prepared. Methyl-phenyl-piperidin-4-yl-amine (101 mg, 0.053 mmol, 5 eq) and HOBt (32 mg, 0.213 mmol, 20 eq.) were packed in Kimble reactor, then 400 μL of the solution of 25-imidazo-carbonyl-21,23-acetonide-rifabutin (I-2) in DCM (corresponding to 10 mg, 0.010 mmol, 1 eq of 25-imidazo-carbonyl-21,23-acetonide-rifabutin) were added. The reaction was stirred at rt. After 5 days, the DCM was evaporated and 600 μL of THF follow by 600 μL of a 0.5N solution of Camphor Sulfonic in water were then added to the mixture. The reaction was stirred at rt. After, 16 h, the mixture was deposited on Column PoraPak (Rxn RP 6CC) equilibrated with water. The product was eluted by 5 mL of water, 5 mL of a saturated aqueous solution of NaHCO₃, 5 mL of a solution of a solution of water/ACN (90/10), 5 mL of water and 10 mL of ACN. The 10 mL of ACN were collected, frozen and freeze-dried to give the desired product Compound 4 as a purple powder.

¹H NMR (300 MHz, CDCl3): δ (ppm) 0.00 (d, J=7.0 Hz, 3H), 0.61 (d, J=6.6 Hz, 3H), 0.86 (d, J=6.8 Hz, 3H), 0.96 (d, J=6.5 Hz, 6H), 1.05 (d, J=6.7 Hz, 3H), 1.47-2.14 (m, 12H), 1.78 (s, 3H), 2.06 (s, 3H), 2.26-245 (m, 3H), 2.32 (s, 3H), 2.63-2.89 (m, 4H), 2.74 (s, 3H), 2.92-3.20 (m, 6H), 3.40 (d, J=6.1 Hz, 1H), 3.63-3.79 (m, 2H), 3.85 (s, 1H), 4.09-4.38 (m, 2H), 4.59 (d, J=10.4 Hz, 1H), 4.73 (brs, 1H), 4.96-5.13 (m, 1H), 6.06 (dd, J=15.6, 6.8 Hz, 1H), 6.17 (d, J=12.3 Hz, 1H), 6.26 (dd, J=10.3, 0.8 Hz, 1H), 6.44 (dd, J=15.6, 10.3 Hz, 1H), 6.72-6.85 (m, 3H), 7.20-7.30 (m, 2H), 8.24 (s, 1H), 8.92 (s, 1H), 14.85 (s, 1H). ¹³C NMR (75 MHz, CDCl3): δ (ppm) 7.7, 8.9, 10.8, 11.6, 17.6, 20.4, 20.9, 21.7, 25.8, 29.1, 31.6, 33.3, 35.3, 36.1, 37.7, 37.8, 38.4, 44.2, 51.4, 51.5, 56.9, 57.1, 66.3, 73.1, 74.7, 77.3, 80.2, 94.4, 104.9, 107.2, 108.6, 112.0, 114.0, 114.1, 114.2, 115.8, 117.6, 124.4, 125.3, 129.3, 130.7, 133.3, 141.2, 142.2, 143.6, 149.8, 155.5, 156.1, 168.4, 168.5, 171.6, 181.4, 192.6.

Table 5 below summarizes exemplary compounds of the invention (R¹ and R² groups shown) that were prepared according to the protocol of Example 3 above. Data for compounds 1-17 given in Table 5 were obtained following the analytic method B. Data for compounds (20, 21, 24, 25, 29, 30, 32, 36-38, 41, 43, 44, 46, and 48-84 were obtained following the analytic method A (*: 10 min analysis; **: 30 min analysis). Where the starting amine was originally prepared as a hydrochloride, an excess of TEA was added prior to the addition of 1-2 to neutralize the hydrochloride salt.

TABLE 5
Compounds of the Invention (R¹ and R² groups shown)

| Cpd | Structure | MW | Rt | [M + H]+ |
|---|---|---|---|---|
| 1 | 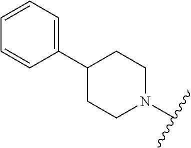 [SMILES: *N1CCC(c2ccccc2)CC1] | 991.53 | 20.64 | 992.72 |
| 2 | 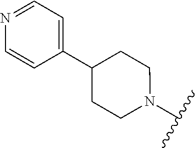 [SMILES: *N1CCC(c2ccncc2)CC1] | 992.52 | 16.47 | 993.69 |
| 3 | 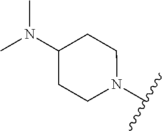 [SMILES: *N1CCC(N(C)C)CC1] | 958.54 | 15.14 | 959.68 |
| 4 | 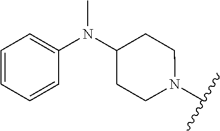 [SMILES: *N1CCC(N(C)c2ccccc2)CC1] | 1020.55 | 20.58 | 1021.76 |
| 5 | 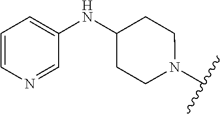 [SMILES: *N1CCC(Nc2cccnc2)CC1] | 1007.53 | 16.02 | 1008.75 |
| 6 | 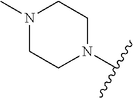 [SMILES: *N1CCN(C)CC1] | 930.51 | 15.32 | 931.73 |
| 7 | 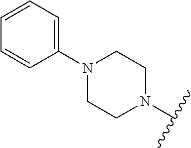 [SMILES: *N1CCN(c2ccccc2)CC1] | 992.52 | 19.50 | 993.77 |
| 8 | 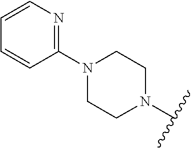 [SMILES: *N1CCN(c2ccccn2)CC1] | 993.52 | 17.90 | 994.73 |
| 9 | 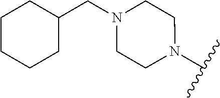 [SMILES: *N1CCN(CC2CCCCC2)CC1] | 1012.58 | 22.87 | 1013.85 |
| 10 | 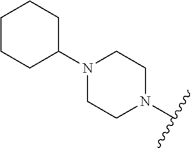 [SMILES: *N1CCN(C2CCCCC2)CC1] | 998.57 | 19.99 | 999.83 |
| 11 | 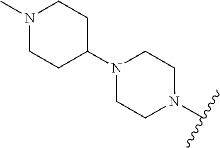 [SMILES: *N1CCN(C2CCN(C)CC2)CC1] | 1013.58 | 14.52 | 1014.84 |
| 12 | 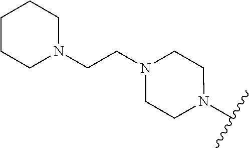 [SMILES: *N1CCN(CCN2CCCCC2)CC1] | 1027.59 | 15.69 | 1028.79 |
| 13 | 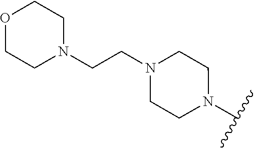 [SMILES: *N1CCN(CCN2CCOCC2)CC1] | 1029.57 | 14.48 | 1030.76 |
| 14 | 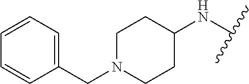 [SMILES: *NC1CCN(Cc2ccccc2)CC1] | 1020.55 | 18.45 | 1021.79 |
| 15 | 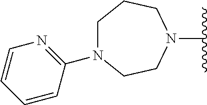 [SMILES: *N1CCCN(c2ccccn2)CC1] | 1007.53 | 17.70 | 1008.78 |
| 16 | 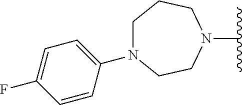 [SMILES: *N1CCCN(c2ccc(F)cc2)CC1] | 1024.53 | 19.28 | 1025.78 |
| 17 | 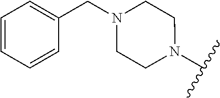 [SMILES: *N1CCN(Cc2ccccc2)CC1] | 1006.54 | 19.47 | 1007.73 |
| 20** | 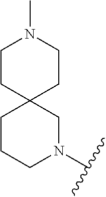 [SMILES: *N1CCCC2(CCN(C)CC2)C1] | 998.57 | 9.73 | 999.59 |
| 21** | 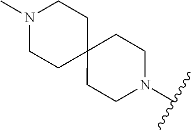 [SMILES: *N1CCC2(CC1)CCN(C)CC2] | 998.57 | 9.14 | 999.49 |
| 24** | 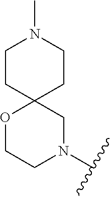 [SMILES: *N1CCOC2(CCN(C)CC2)C1] | 1000.55 | 9.35 | 1001.59 |
| 25** | 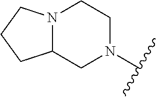 [SMILES: *N1CCN2CCCC2C1] | 956.53 | 9.22 | 957.51 |
| 29* | 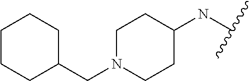 [SMILES: *NC1CCN(CC2CCCCC2)CC1] | 1026.60 | 5.58 | 1027.68 |
| 30 | 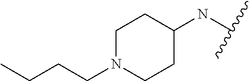 [SMILES: *NC1CCN(CCCC)CC1] | 986.57 | 2.77 | 987.67 |
| 32* | 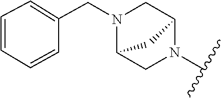 [SMILES: *N1C[C@@H]2C[C@H]1CN2Cc1ccccc1] | 1018.54 | 4.68 | 1019.68 |
| 36* | 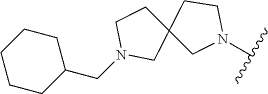 [SMILES: *N1CCC2(CCN(CC3CCCCC3)C2)C1] | 1052.62 | 5.39 | 1053.77 |
| 37** | 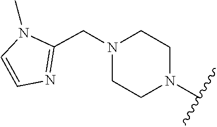 [SMILES: *N1CCN(Cc2nccn2C)CC1] | 1010.55 | 9.50 | 1011.43 |
| 38** | 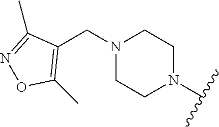 [SMILES: *N1CCN(Cc2c(C)noc2C)CC1] | 1025.55 | 11.97 | 1026.53 |
| 41* | 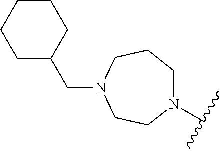 [SMILES: *N1CCCN(CC2CCCCC2)CC1] | 1026.60 | 4.23 | 1027.48 |
| 43* | 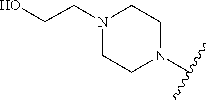 [SMILES: *N1CCN(CCO)CC1] | 960.52 | 3.48 | 961.56 |
| 44* | 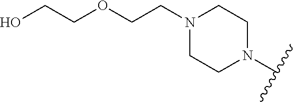 [SMILES: *N1CCN(CCOCCO)CC1] | 1004.55 | 3.30 | 1005.44 |
| 46** | 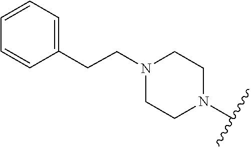 [SMILES: *N1CCN(CCc2ccccc2)CC1] | 1020.56 | 12.02 | 1021.58 |
| 48* | 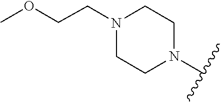 [SMILES: *N1CCN(CCOC)CC1] | 974.54 | 3.56 | 976.45 |
| 49** | 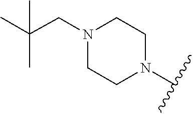 [SMILES: *N1CCN(CC(C)(C)C)CC1] | 986.57 | 13.84 | 987.64 |
| 50** | 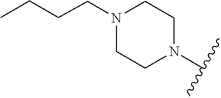 [SMILES: *N1CCN(CCCC)CC1] | 972.56 | 10.37 | 973.65 |
| 51** | 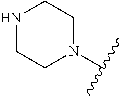 [SMILES: *N1CCNCC1] | 916.49 | 13.12 | 917.45 |
| 52** | 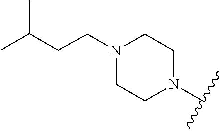 [SMILES: *N1CCN(CCC(C)C)CC1] | 986.57 | 15.48 | 987.67 |
| 53 | 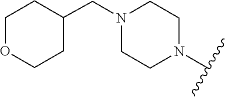 [SMILES: *N1CCN(CC2CCOCC2)CC1] | 1014.57 | 2.32 | 1015.55 |
| 54 | 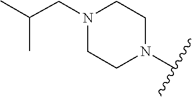 [SMILES: *N1CCN(CC(C)C)CC1] | 972.56 | 2.01 | 973.60 |
| 55** | 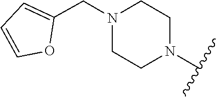 [SMILES: *N1CCN(Cc2ccco2)CC1] | 996.52 | 11.86 | 997.49 |
| 56** | 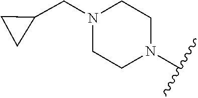 [SMILES: *N1CCN(CC2CC2)CC1] | 970.54 | 9.57 | 971.70 |
| 57** | 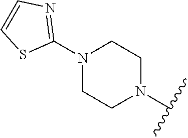 [SMILES: *N1CCN(c2nccs2)CC1] | 999.48 | 12.40 | 1000.54 |
| 58 | 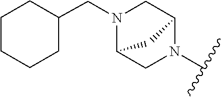 [SMILES: *N1C[C@@H]2C[C@H]1CN2CC1CCCCC1] | 1024.59 | 1.94 | 1025.6 |
| 59** | 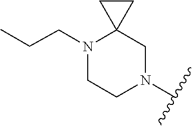 [SMILES: *N1CCN(CCC)C2(CC2)C1] | 984.56 | 15.05 | 985.72 |
| 60* | 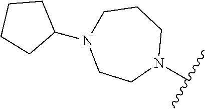 [SMILES: *N1CCCN(C2CCCC2)CC1] | 998.57 | 3.66 | 999.54 |
| 61* | 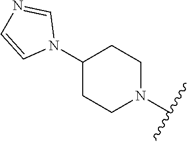 [SMILES: *N1CCC(n2ccnc2)CC1] | 981.52 | 3.62 | 982.55 |
| 62* | 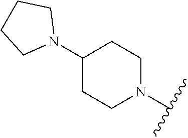 [SMILES: *N1CCC(N2CCCC2)CC1] | 984.56 | 3.41 | 985.60 |
| 63* | 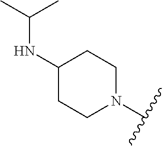 [SMILES: *N1CCC(NC(C)C)CC1] | 972.56 | 3.45 | 973.65 |
| 64** | 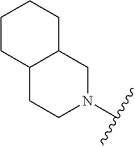 [SMILES: *N1CCC2CCCCC2C1] | 969.55 | 16.21 | 970.60 |
| 65* | 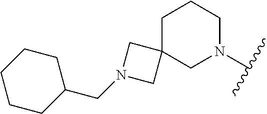 [SMILES: *N1CCCC2(C1)CN(CC1CCCCC1)C2] | 1052.62 | 6.47 | 1053.64 |
| 66** | 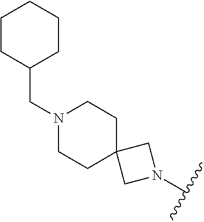 [SMILES: *N1CC2(CCN(CC3CCCCC3)CC2)C1] | 1052.62 | 18.16 | 1053.64 |
| 67* | 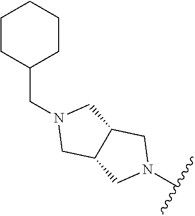 [SMILES: *N1C[C@@H]2CN(CC3CCCCC3)C[C@@H]2C1] | 1038.60 | 6.37 | 1039.57 |
| 68 | 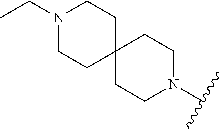 [SMILES: *N1CCC2(CC1)CCN(CC)CC2] | 1012.59 | 2.49 | 1013.66 |
| 69* | 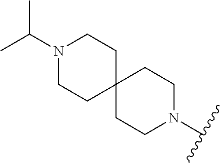 [SMILES: *N1CCC2(CC1)CCN(C(C)C)CC2] | 1026.60 | 2.58 | 1027.75 |
| 70* | 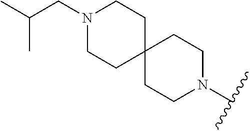 [SMILES: *N1CCC2(CC1)CCN(CC(C)C)CC2] | 1040.62 | 6.12 | 1041.74 |
| 71* | 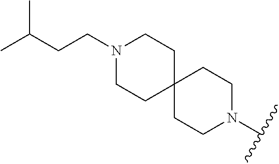 [SMILES: *N1CCC2(CC1)CCN(CCC(C)C)CC2] | 1054.64 | 6.48 | 1055.74 |
| 72* | 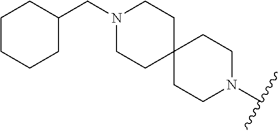 [SMILES: *N1CCC2(CC1)CCN(CC1CCCCC1)CC2] | 1080.65 | 6.37 | 1081.61 |
| 73** | 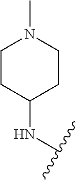 [SMILES: *NC1CCN(C)CC1] | 944.53 | 13.12 | 945.54 |
| 74** | 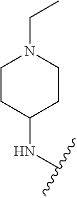 [SMILES: *NC1CCN(CC)CC1] | 958.54 | 13.62 | 959.63 |
| 75* | 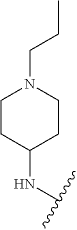 [SMILES: *NC1CCN(CCC)CC1] | 972.56 | 5.56 | 973.72 |
| 76** | 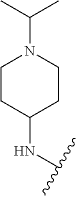 [SMILES: *NC1CCN(C(C)C)CC1] | 972.56 | 14.78 | 973.57 |
| 77** | 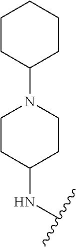 [SMILES: *NC1CCN(C2CCCCC2)CC1] | 1012.59 | 10.34 | 1013.68 |
| 78* | 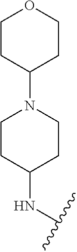 [SMILES: *NC1CCN(C2CCOCC2)CC1] | 1014.57 | 5.86 | 1015.70 |
| 82** | 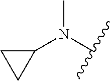 [SMILES: *N(C)C1CC1] | 901.48 | 13.52 | 902.51 |
| 83** | 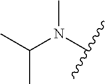 [SMILES: *N(C)C(C)C] | 903.50 | 14.18 | 904.51 |

Additional analytical data (NMR) for Compound 9 is given below:

Compound 9 ¹H NMR (300 MHz, CDCl3): δ (ppm) −0.12 (d, J=6.9 Hz, 3H), 0.54 (d, J=6.6 Hz, 3H), 0.79 (d, J=6.8 Hz, 3H), 0.84-0.93 (m, 2H), 0.96 (d, J=6.8 Hz, 3H), 1.00-1.27 (m, 10H), 1.30-1.42 (m, 1H), 1.45-1.83 (m, 7H), 1.70 (s, 3H), 1.96 (s, 3H), 2.00-2.11 (m, 1H), 2.22-2.40 (m, 3H), 2.27 (s, 3H), 2.54-2.84 (m, 10H), 2.90-3.41 (m, 10H), 3.01 (s, 3H), 3.48-3.72 (m, 3H), 4.81 (d, J=10.5 Hz, 1H), 5.08 (dd, J=12.6, 7.4 Hz, 1H), 5.91 (dd, J=15.6, 6.7 Hz, 1H), 6.10 (d, J=12.6 Hz, 1H), 6.18 (d, J=9.9 Hz, 1H), 6.33 (dd, J=15.6, 10.2 Hz, 1H), 8.18 (s, 1H), 9.33 (brs, 1H), 14.72 (s, 1H).

¹³C NMR (75 MHz, CDCl3): δ (ppm) 7.7, 8.8, 10.8, 11.25, 17.3, 20.4, 21.0, 21.9, 24.8, 26.0, 26.4, 31.8, 32.9, 34.3, 37.6, 38.4, 51.5, 51.8, 52.7, 56.9, 58.7, 64.7, 65.2, 72.5, 73.4, 76.7, 80.9, 92.6, 104.4, 107.3, 108.7, 111.8, 114.6, 115.1, 124.3, 124.8, 131.1, 133.1, 140.6, 142.0, 144.3, 156.0, 167.9, 168.2, 170.7, 171.4, 181.5, 192.7.

### Example 4: Preparation of the Coupling Amine Used for Synthesis of Compound 67

### Synthesis of cis-2-Boc-Hexahydropyrrol[3,4-c]pyrrole dihydrochloride

Amine groups were functionalized by alkylation and/or Boc deprotection as necessary prior to conjugation to Intermediate I-2 as described herein.

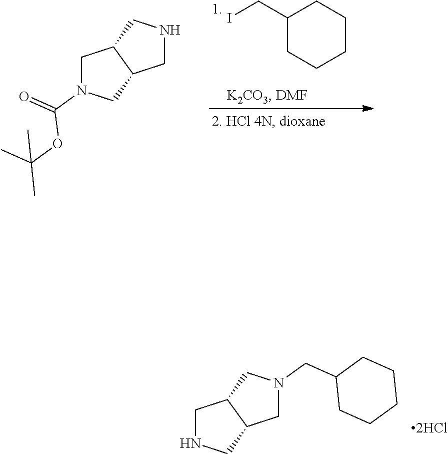

Step 1: In a glass vial, tert-butyl (3aR,6aS)-2,3,3a,4,6,6a-hexahydro-1H-pyrrolo[3,4-c]pyrrole-5-carboxylate (150 mg, 0.707 mmol), K₂CO₃ (146 mg, 1.06 mmol) and iodomethylcyclohexane (238 mg, 1.06 mmol) were diluted with 5 ml of DMF and stirred at 70° C. for 36 h. The mixture was diluted with H₂O, then extracted twice with EtOAc. Then the combined organic layers were washed with NaCl brine 5 times to remove the DMF. The organic phase was dried over MgSO₄ and concentrated to dryness. The crude product was purified by flash chromatography with DCM-MeOH (100-0 to 95-5). The product was obtained as a translucid solid, 197 mg, 90% yield.

¹H NMR (300 MHz, CDCl₃): δ (ppm) 3.68-3.43 (m, 2H), 3.37-3.13 (m, 2H), 2.93-2.72 (m, 2H), 2.72-2.57 (m, 2H), 2.41-2.28 (m, 2H), 2.23 (d, J=7.2 Hz, 2H), 1.89-1.58 (m, 5H), 1.46 (s, J=5.5 Hz, 9H), 1.40 (m, 1H), 1.32-1.03 (m, 3H), 0.99-0.75 (m, 2H).

Step 2: The product from Step 1 was dissolved in 1,4-dioxane (2 ml) then HCl (4 N in dioxane, 4 ml) was added dropwise and the mixture was stirred for 18 h at RT. After this time the suspension was concentrated to dryness to obtain a white solid, 180 mg, quantitative yield.

The amine coupling groups of Compounds 36, 52, 58, 59, 65, 66, 67, 70, 71 and 72 were prepared as described in this Example.

### Example 5: Preparation of the Coupling Amine Used for Synthesis of Compound 78

### Synthesis of 1-tetrahydropyran-4-ylpiperidin-4-amine dihydrochloride

Amine groups were functionalized by reductive amination and/or Boc deprotection as necessary prior to conjugation to Intermediate I-2 as described herein.

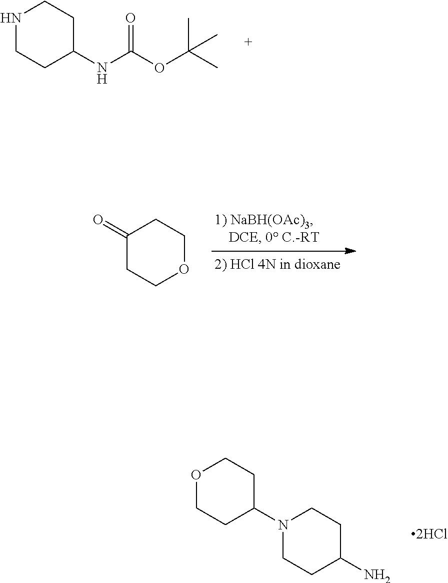

Step 1: A round-bottom flask was charged with tert-butyl N-(4-piperidyl)carbamate (700 mg, 3.49 mmol) and DCE (15 ml), then the mixture was brought to 0° C. The mixture was stirred at this temperature for 5 minutes then tetrahydropyran-4-one (0.389 ml, 4.19 mmol, 1.2 eq) was added and the mixture was stirred at this temperature for 1 h. After this time, sodium triacetoxyborohydride (1.5 g, 7 mmol, 2 eq) was added and the mixture was stirred in the melting ice bath for 18 h. After this time, a minimum amount of MeOH was added and the mixture was concentrated to dryness then EtOAc (20 ml) was added and the organic phase was washed with H₂O and NaCl brine, dried over MgSO₄ and concentrated to dryness to obtain a colorless oil, 905 mg, 91% yield.

Step 2: The product from the previous step was dissolved in 1,4-dioxane (5 ml) then HCl (4 N in dioxane, 8 ml) was added dropwise and the mixture was stirred overnight at RT.

After this time the suspension was concentrated to dryness to obtain a white solid, 810 mg, 90% yield.

¹H NMR (300 MHz, DMSO) δ 11.26 (d, J=60.4 Hz, 1H), 8.59 (s, 3H), 4.01-3.89 (m, 2H), 3.59-3.46 (m, 2H), 3.35-3.22 (m, 4H), 3.10-2.90 (m, 2H), 2.29-2.11 (m, 2H), 2.11-1.93 (m, 4H), 1.86-1.63 (m, 2H).

The amine coupling groups of Compounds 69 and 78 were prepared as described in this Example.

### Example 6: Antibacterial Activity

MIC values were determined by broth microdilution method according to the CLSI guidelines with the following exceptions: MIC against A. baumannii was performed in RPMI medium supplemented with 10% FCS and MIC against M. abscessus was performed in Middlebrook 7H9 broth supplemented with Middlebrook ADC growth supplement (10%; see column “M. abscessus 7H9”). MIC forM abscessus was determined a second time in standard cation adjusted Mueller Hinton broth without growth supplement (see colum “M. abscessus MH”). All other MIC were performed in standard cation adjusted Mueller Hinton broth, supplemented with Middlebrook ADC growth supplement (5%) for the slow growing mycobacteria (M. xenopi, M. kansasii and M. avium).

The following procedure applies to all species tested, except forM xenopi. From a fresh culture plate (from overnight to several days depending on the bacteria), cells were resuspended in 0.9% (w/v) saline solution and bacterial inoculum was prepared in the respective testing medium with 5×10⁵ CFU/mL. For M. xenopi, the inoculum was prepared from a fresh liquid culture (7H9+ADC). The appropriate volumes of a 10 mg/mL compound solution were directly dispensed in the 96-well assay plate using a digital dispenser to create two-fold dilution series from 32 to 0.002 μg/mL final concentration for A. baumannii and S. aureus; from 16 to 0.016 μg/mL for M. abscessus and M. avium; and from 1 to 6×10⁻⁵ μg/mL for M. kansasii and M. xenopi. 100 μL of bacterial suspension was finally added to the compounds. Plates were covered and incubated without shaking at 35° C. for 20 hours for A. baumanii and S. aureus; at 30° C. for 4 days for M. abscessus; and at 37° C. for 7 days (at least) for M. kansasii (covered with aluminum foil to avoid light exposure), M. avium and M. xenopi. Every experiment contained an antibiotic as quality control. MIC was determined visually as the lowest concentration of a compound that prevents visible growth of the bacteria.

The in vitro activity of compounds described herein was determined against S. aureus (UAMS-1625 strain), A. baumannii (HUMC1 strain), M. abscessus (ATCC 19977), M. kansasii (ATCC 12478), M. avium (ATCC 25291), and M. xenopi (ATCC 19250). The MIC values, given in μg/mL, are given in Table 6, below.

TABLE 6
In vitro activity of described compounds against A. baumannii, S. aureus,
M. abscessus, M. kansasii, M. xenopi and M. avium

| Compound | | | M. abscessus | M. abscessus | | | |
|---|---|---|---|---|---|---|---|
| Number | S. aureus | A. baumannii | 7H9 | MH | M. kansasii | M. xenopi | M. avium |
| Rifabutin | 0.016 | ≤0.002 | 8 | 16 | | | |
| 1 | 0.5 | 0.25 | 0.125 | 0.5 | 0.004 | 0.03 | 0.125 |
| 2 | 0.125 | 0.016 | 0.125 | 0.25 | 0.0005 | 0.002 | 0.06 |
| 3 | 0.03 | ≤0.002 | 0.5 | 4 | | | |
| 4 | 0.5 | 0.125 | 0.25 | 1 | 0.002 | 0.016 | 0.25 |
| 5 | 0.125 | 0.004 | 1 | 4 | | | |
| 6 | 0.06 | 0.06 | 1 | 4 | | | |
| 7 | 0.5 | 0.125 | 0.125 | 0.5 | 0.001 | 0.008 | 0.25 |
| 8 | 0.25 | 0.25 | 0.125 | 0.5 | 0.0005 | 0.008 | 0.25 |
| 9 | 0.5 | 0.5 | 0.06 | 0.125 | 0.0002 | 0.016 | 0.125 |
| 10 | 0.25 | 0.06 | 0.06 | 0.06 | 0.0002 | 0.002 | 0.06 |
| 11 | 0.125 | ≤0.002 | 0.5 | 4 | | | |
| 12 | 0.125 | ≤0.002 | 0.5 | 1 | | | |
| 13 | 0.06 | ≤0.002 | 0.5 | 0.5 | | | |
| 14 | 0.125 | 0.004 | 0.25 | 0.25 | 0.0005 | 0.008 | 0.25 |
| 15 | 0.06 | 0.008 | 0.5 | 1 | | | |
| 16 | 0.25 | 0.03 | 1 | 2 | | | |
| 17 | 0.25 | 0.125 | 0.125 | 1 | 0.0005 | 0.004 | 0.125 |
| 20 | 0.06 | ≤0.016 | | 1 | | | |
| 21 | 0.06 | ≤0.016 | | 2 | 0.125 | 0.25 | 0.5 |
| 24 | 0.06 | ≤0.016 | | 2 | | | |
| 25 | 0.03 | ≤0.016 | | 2 | | | |
| 29 | 0.125 | ≤0.016 | | 0.25 | | | |
| 30 | 0.06 | ≤0.016 | | 0.5 | 0.016 | 0.06 | 0.125 |
| 32 | 0.25 | 0.03 | | 0.5 | | | |
| 36 | 0.25 | 0.06 | | 0.125 | | | |
| 37 | 0.03 | ≤0.016 | | 2 | | | |
| 38 | 0.125 | ≤0.016 | | 1 | | | |
| 41 | 1 | 0.06 | | 0.125 | | | |
| 43 | 0.06 | ≤0.016 | | 8 | | | |
| 44 | 0.03 | ≤0.016 | | 4 | | | |
| 46 | 0.25 | 0.25 | | 0.5 | | | |
| 48 | 0.06 | ≤0.016 | | 1 | | | |
| 49 | 0.5 | 1 | | 0.125 | | | |
| 50 | 0.125 | 0.03 | | 0.25 | 0.008 | 0.008 | 0.125 |
| 51 | 0.016 | ≤0.001 | | 4 | | | |
| 52 | 0.25 | 0.06 | | 0.125 | | | |
| 53 | 0.125 | 0.004 | | 0.125 | | | |
| 54 | 0.125 | 0.03 | | 0.06 | | | |
| 55 | 0.125 | ≤0.016 | | 2 | | | |
| 56 | 0.125 | ≤0.016 | | 0.25 | 0.004 | 0.008 | 0.06 |
| 57 | 0.06 | 0.03 | | 0.5 | | | |
| 58 | 0.125 | 0.03 | | 0.125 | | | |
| 59 | 0.25 | 0.03 | | 0.125 | | | |
| 60 | 0.06 | ≤0.016 | | 0.25 | | | |
| 61 | 0.03 | ≤0.016 | | 4 | | | |
| 62 | 0.06 | ≤0.016 | | 1 | | | |
| 63 | 0.06 | ≤0.016 | | 2 | 0.125 | 0.25 | 0.5 |
| 64 | 1 | 0.5 | | 4 | | | |
| 65 | 0.5 | 0.004 | | 0.125 | | | |
| 66 | 0.125 | 0.004 | | 0.125 | | | |
| 67 | 1 | 0.06 | | 0.125 | 0.003 | 0.004 | 0.016 |
| 68 | 0.06 | 0.003 | | 1 | | | |
| 69 | 0.125 | 0.002 | | 1 | | | |
| 70 | 0.125 | 0.004 | | 0.25 | | | |
| 71 | 0.25 | 0.008 | | 0.25 | | | |
| 72 | 0.25 | 0.06 | | 0.25 | 0.0004 | 0.008 | 0.03 |
| 73 | 0.03 | 0.001 | | 1 | | | |
| 74 | 0.03 | ≤0.001 | | 2 | | | |
| 75 | 0.06 | 0.001 | | 1 | | | |
| 76 | 0.03 | ≤0.001 | | 2 | | | |
| 77 | 0.06 | ≤0.016 | | 1 | 0.03 | 0.06 | 0.25 |
| 78 | 0.06 | ≤0.001 | | 2 | | | |
| 82 | 0.06 | 0.008 | | 4 | | | |
| 83 | 0.125 | 0.016 | | 2 | | | |

1. A compound of Formula I or a pharmaceutically acceptable salt, tautomer, solvate, hydrate or enantiomer thereof: 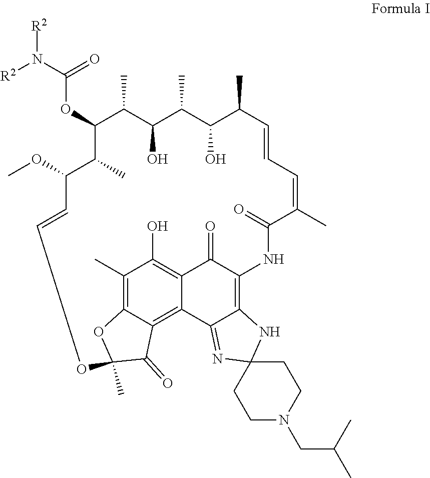
wherein:
R¹ and R² are each independently selected from —H, —C₁-C₆ alkyl, —C₃-C₁₀ cycloalkyl, C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, and —C₁-C₆ alkylene-(5- to 10-membered heteroaryl), wherein each alkyl, cycloalkyl, heterocycloalkyl, aryl, or heteroaryl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —CN, —C₁-C₆ alkoxy, —C₁-C₆ alkyl, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl); wherein each cycloalkyl, heterocycloalkyl, aryl, or heteroaryl is optionally substituted with one or more —OH, halogen, or C₁-C₆ alkyl;
R⁴ is independently, at each occurrence, —C₁-C₆ alkyl, —OR⁵, —C₁-C₆ alkylene-OR⁵, —NR⁶R⁷, —C₁-C₆ alkylene-NR⁶R⁷, —C₃-C₁₀ cycloalkyl, —C₁-C₆ alkylene-C₃-C₁₀ cycloalkyl, 3- to 15-membered heterocycloalkyl, —C₁-C₆ alkylene-(3- to 15-membered heterocycloalkyl), —C₆-C₁₀ aryl, —C₁-C₆ alkylene-C₆-C₁₀ aryl, 5- to 10-membered heteroaryl, or —C₁-C₆ alkylene-(5- to 10-membered heteroaryl), wherein each alkylene is optionally substituted with one or more oxo, and wherein each alkyl, cycloalkyl, heterocycloalkyl, aryl or heteroaryl is optionally substituted with one or more R⁸;
R⁵ is independently, at each occurrence, —H, —C₁-C₆ alkyl, or —C₁-C₆ alkylene-OH;
R⁶ and R⁷ are each independently, at each occurrence, —H, —C₁-C₆ alkyl, —C₃-C₁₀ cycloalkyl, 3- to 15-membered heterocycloalkyl, C₆-C₁₀ aryl, or 5- to 10-membered heteroaryl; and
R⁸ is independently, at each occurrence, —OH, halogen, or —C₁-C₆ alkyl.

2. The compound of claim 1, wherein R¹ and R² are each independently selected from —H, —C₁-C₄ alkyl, —C₃-C₁₀ cycloalkyl, C₁-C₄ alkylene-C₃-C₁₀ cycloalkyl, -5- to 15-membered heterocycloalkyl, and —C₁-C₄ alkylene-(5- to 15-membered heterocycloalkyl), wherein each alkyl, cycloalkyl, or heterocycloalkyl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄alkylene-(5- to 10-membered heteroaryl); wherein each cycloalkyl, heterocycloalkyl, aryl, or heteroaryl is optionally substituted with one or more —OH, halogen, or C₁-C₄ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 10-membered heterocycloalkyl, C₆-C₁₀ aryl, or 5- to 10-membered heteroaryl; and
R⁸ is independently, at each occurrence, —OH, halogen, or —C₁-C₄ alkyl.

3. The compound of claim 1 or 2, wherein R¹ and R² are each independently selected from —H, -5- to 15-membered heterocycloalkyl, and —C₁-C₄ alkylene-(5- to 15-membered heterocycloalkyl), wherein each heterocycloalkyl is optionally substituted with one or more R³; or
R¹ and R², together with the nitrogen atom to which they are attached, combine to form a 5- to 15-membered heterocycloalkyl, wherein said heterocycloalkyl is optionally substituted with one or more R⁴;
R³ is independently, at each occurrence, —OH, halogen, —C₁-C₄ alkyl, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more —OH, halogen, or C₁-C₄ alkyl;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₃-C₁₀ cycloalkyl, —C₁-C₄ alkylene-C₃-C₁₀ cycloalkyl, 5- to 15-membered heterocycloalkyl, —C₁-C₄ alkylene-(5- to 15-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, aryl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₃-C₁₀ cycloalkyl, 5- to 6-membered heterocycloalkyl, C₆-C₁₀ aryl, or 5- to 10-membered heteroaryl; and
R⁸ is independently, at each occurrence, halogen or —C₁-C₄ alkyl.

4. The compound of any of claims 1 to 3, wherein R¹ and R² are each independently selected from —H, —C₁-C₆ alkyl, —C₃-C₆ cycloalkyl, C₁-C₆ alkylene-C₃-C₆ cycloalkyl, -5- to 6-membered heterocycloalkyl, —C₁-C₆ alkylene-(5- to 6-membered heterocycloalkyl), —C₆-C₁₀ aryl, —C₁-C₆ alkylene-C₆-C₁₀ aryl, 5- to 6-membered heteroaryl, or —C₁-C₆ alkylene-(5- to 6-membered heteroaryl), wherein each cycloalkyl, heterocycloalkyl, aryl, or heteroaryl is optionally substituted with one or more R³.

5. The compound of any of claims 1 to 4, wherein R¹ and R² are each independently selected from —H, -5- to 12-membered heterocycloalkyl, and —C₁-C₄ alkylene-(5- to 12-membered heterocycloalkyl), wherein each heterocycloalkyl is optionally substituted with one or more R³; and
R³ is independently, at each occurrence, —C₁-C₄ alkyl, —C₃-C₁₀ cycloalkyl, —C₁-C₄ alkylene-C₃-C₁₀ cycloalkyl, 5- to 12-membered heterocycloalkyl, —C₁-C₄ alkylene-(5- to 12-membered heterocycloalkyl), —C₆-C₁₀ aryl, —C₁-C₄ alkylene-C₆-C₁₀ aryl, 5- to 10-membered heteroaryl, or —C₁-C₄ alkylene-(5- to 10-membered heteroaryl); wherein each cycloalkyl, heterocycloalkyl, aryl, or heteroaryl is optionally substituted with one or more C₁-C₄ alkyl.

6. The compound of any of claims 1 to 5, wherein R¹ is —H;
R² is independently selected from −5- to 6-membered heterocycloalkyl and —C₁-C₂ alkylene-(5- to 6-membered heterocycloalkyl), wherein each heterocycloalkyl is optionally substituted with one or more R³; and
R³ is independently, at each occurrence, —C₁-C₄ alkyl, —C₃-C₆ cycloalkyl, —C₁-C₂ alkylene-C₃-C₆ cycloalkyl, 5- to 6-membered heterocycloalkyl, —C₁-C₂ alkylene-(5- to 6-membered heterocycloalkyl), —C₆— aryl, —C₁-C₂ alkylene-C₆ aryl, 5- to 6-membered heteroaryl, or —C₁-C₂ alkylene-(5- to 6-membered heteroaryl); wherein each cycloalkyl, heterocycloalkyl, aryl, or heteroaryl is optionally substituted with one or more C₁-C₂ alkyl.

7. The compound of any of claims 1 to 3, wherein R¹ and R², together with the nitrogen atom to which they are attached, combine to form a 5- to 15-membered heterocycloalkyl, wherein said heterocycloalkyl is optionally substituted with one or more R⁴.

8. The compound of any of claims 1 to 3 or 7, wherein R¹ and R², together with the nitrogen atom to which they are attached, combine to form a 5- to 12-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₁₀ cycloalkyl, —C₁-C₄ alkylene-C₅-C₁₀ cycloalkyl, 5- to 12-membered heterocycloalkyl, —C₁-C₄ alkylene-(5- to 12-membered heterocycloalkyl), phenyl, —C₁-C₄ alkylene-phenyl, 5- to 10-membered heteroaryl, or —C₁-C₄ alkylene-(5- to 10-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

9. The compound of any of claims 1 to 3 or 7 to 8, wherein R¹ and R², together with the nitrogen atom to which they are attached, combine to form a 6- to 7-membered heterocycloalkyl, wherein said heterocycloalkyl is optionally substituted with one or more R⁴;
R⁴ is independently, at each occurrence, —C₁-C₆ alkyl, —OR⁵, —C₁-C₄ alkylene-OR⁵, —NR⁶R⁷, —C₁-C₄ alkylene-NR⁶R⁷, —C₅-C₆ cycloalkyl, —C₁-C₄ alkylene-C₅-C₆ cycloalkyl, 5- to 6-membered heterocycloalkyl, —C₁-C₄ alkylene-(5- to 6-membered heterocycloalkyl), phenyl, —C₁-C₄ alkylene-phenyl, 5- to 6-membered heteroaryl, or —C₁-C₄ alkylene-(5- to 6-membered heteroaryl), wherein each alkylene is optionally substituted with one or more oxo; and wherein each alkyl, cycloalkyl, heterocycloalkyl, phenyl or heteroaryl is optionally substituted with one or more R⁸;
R⁵ is independently, at each occurrence, —H, —C₁-C₄ alkyl, or —C₁-C₄ alkylene-OH;
R⁶ and R⁷ are each independently, at each occurrence, —H, —C₁-C₄ alkyl, —C₅-C₆ cycloalkyl, 5- to 6-membered heterocycloalkyl, phenyl, or 5- to 6-membered heteroaryl; and
R⁸ is independently, at each occurrence, halogen or —C₁-C₄ alkyl.

10. The compound according to claim 1, wherein the compound is selected from the group

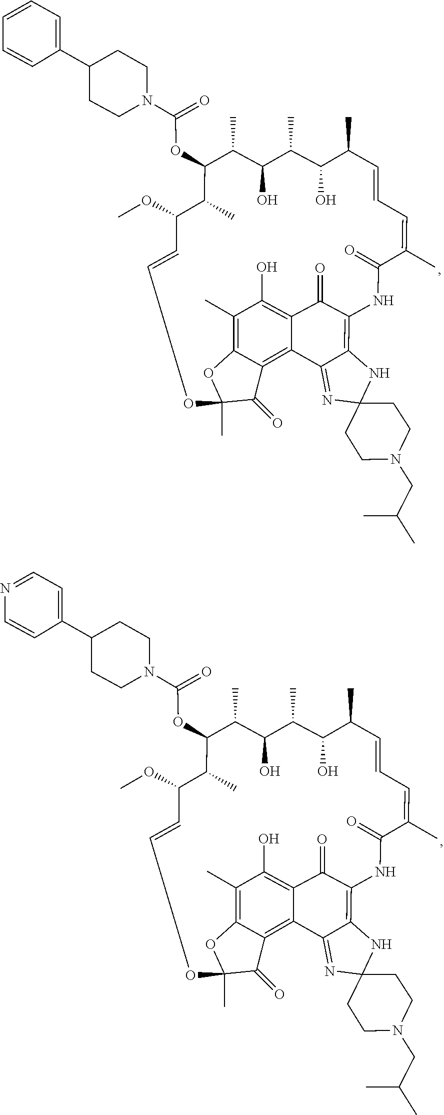 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccncc3)CC2)[C@@H]1C)C4=O]

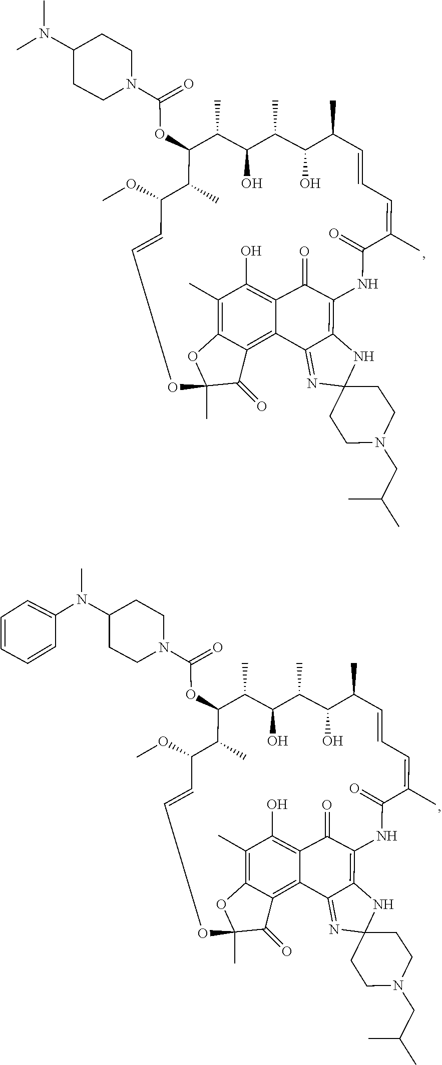

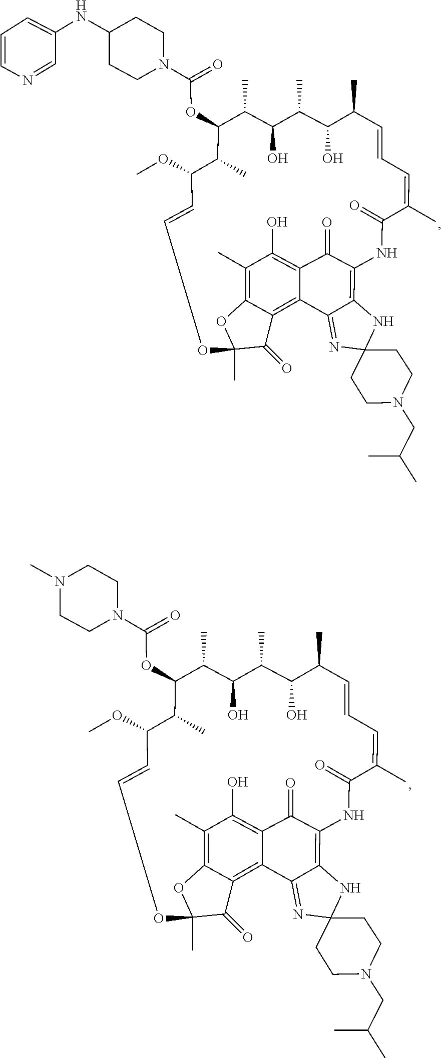

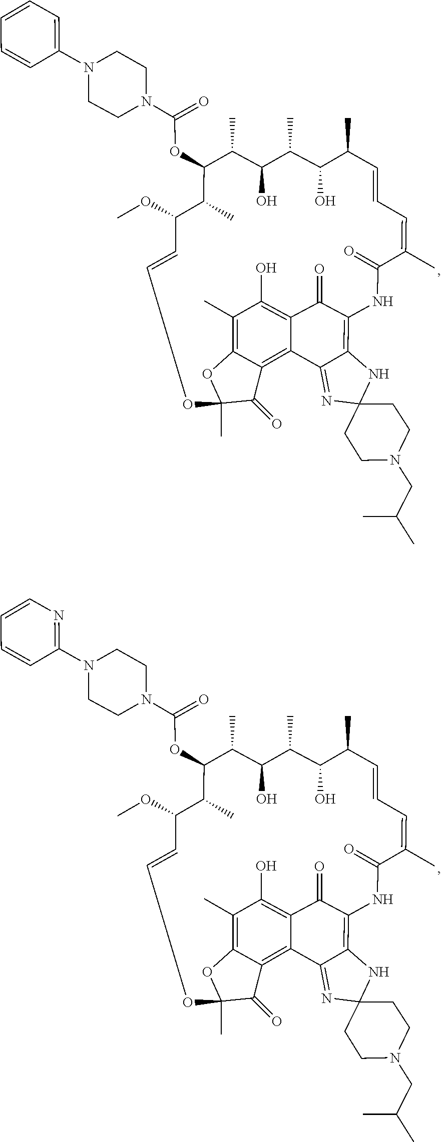 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccn3)CC2)[C@@H]1C)C4=O]

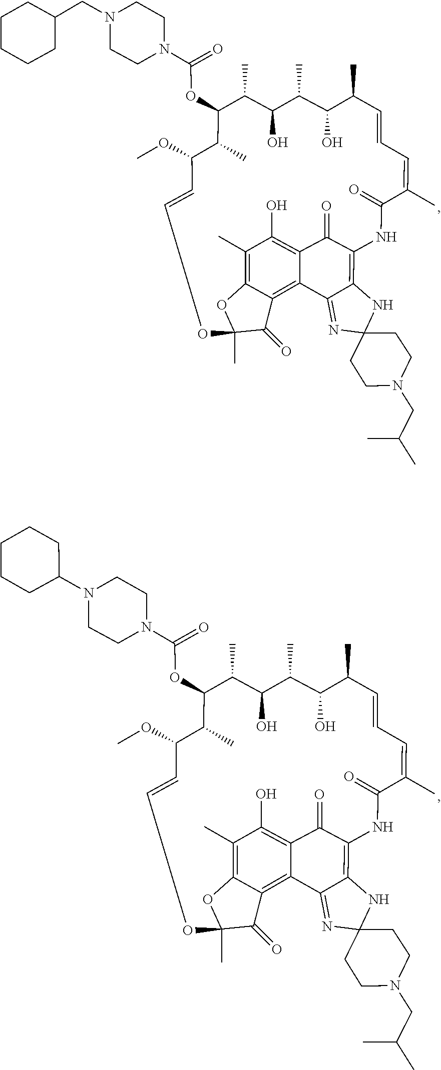 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCCC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCCCC3)CC2)[C@@H]1C)C4=O]

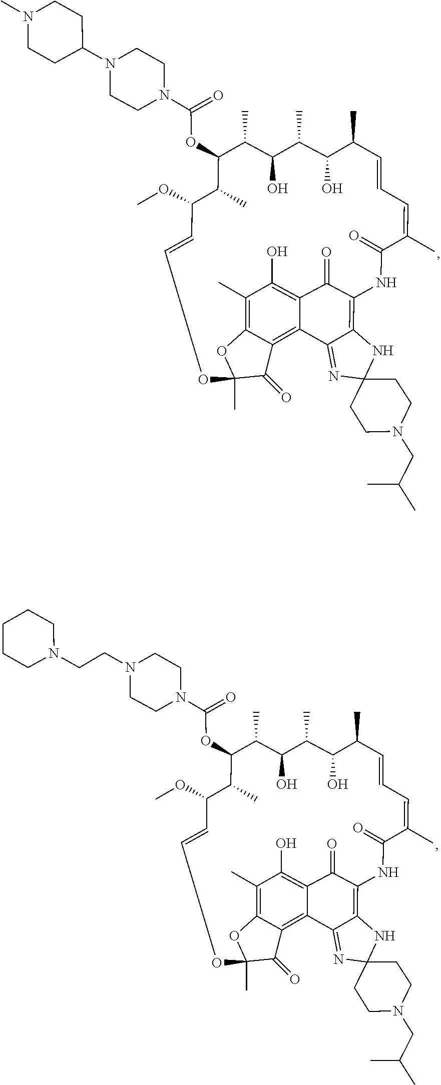

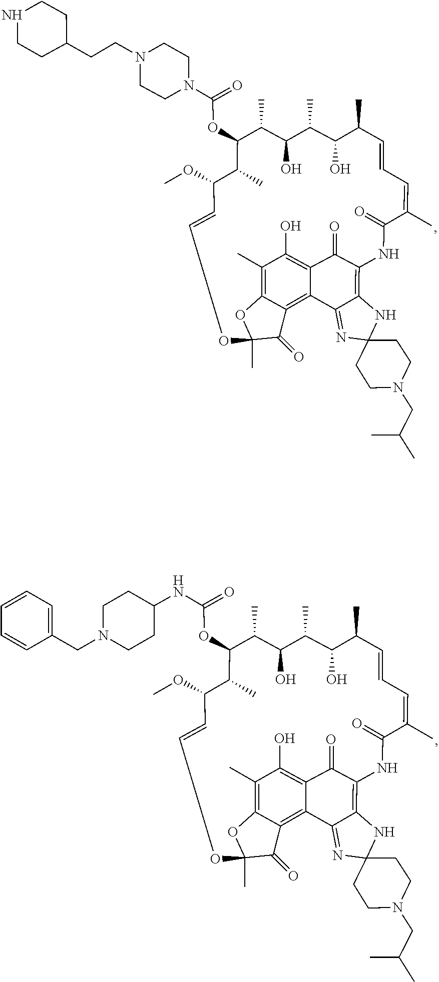 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCC3CCNCC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)CC2)[C@@H]1C)C4=O]

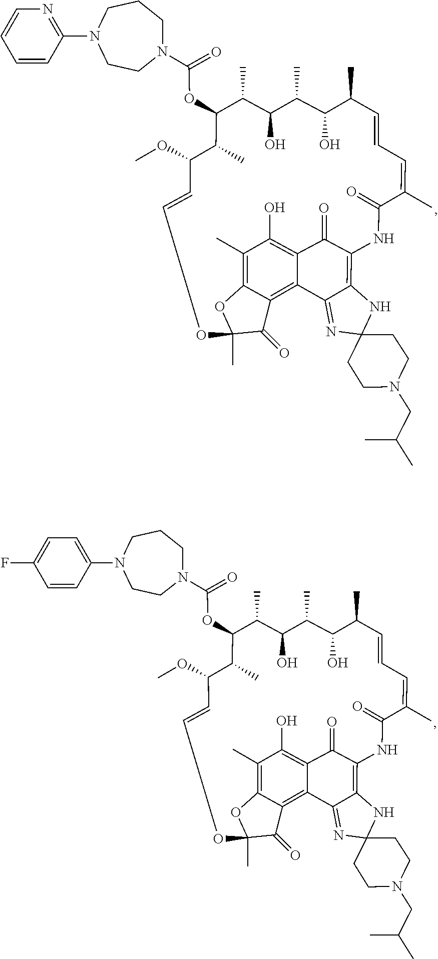 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccccn3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccc(F)cc3)CC2)[C@@H]1C)C4=O]

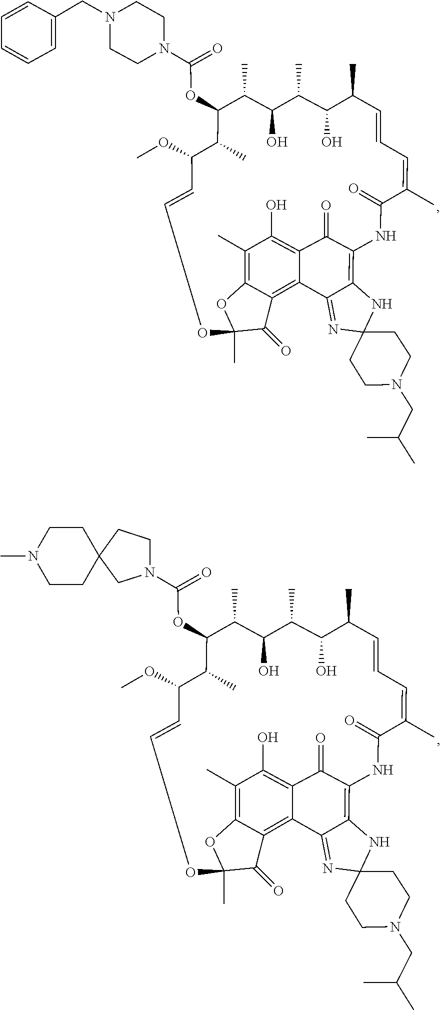

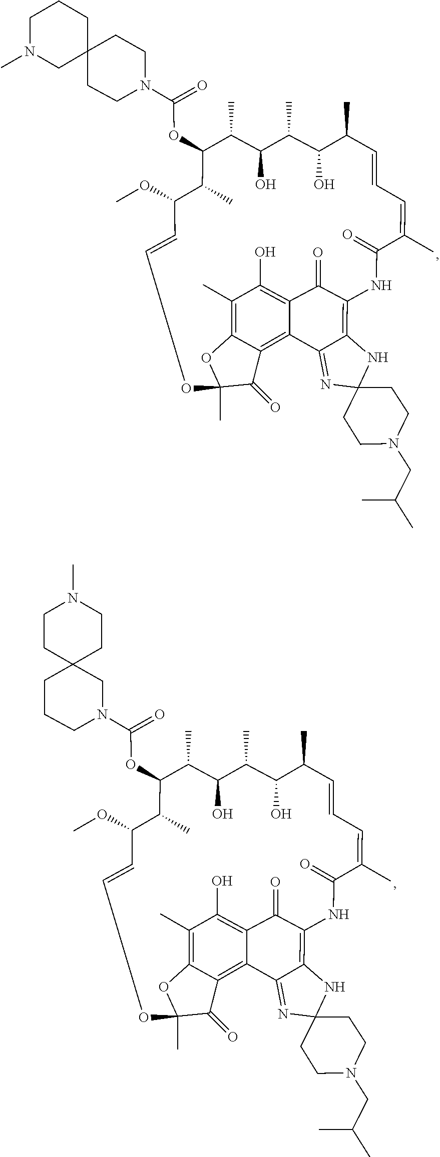

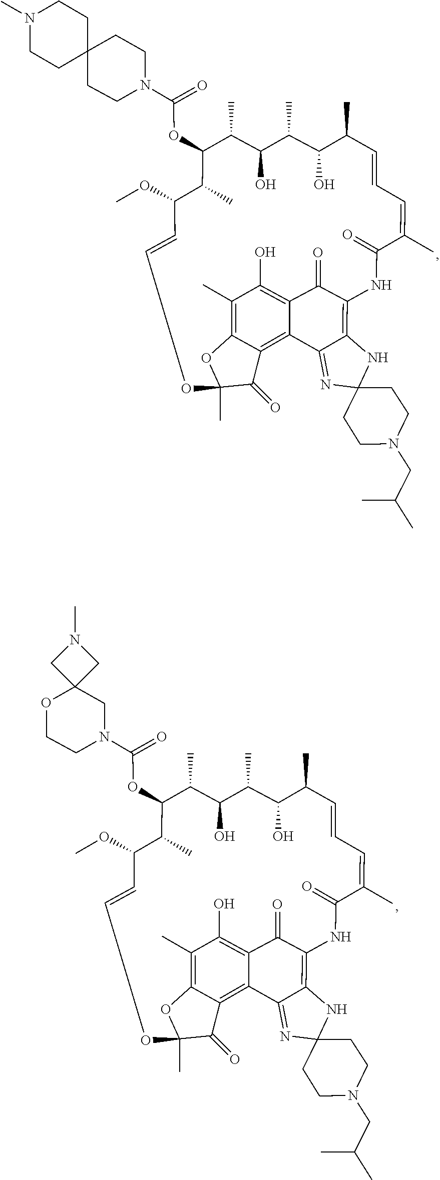

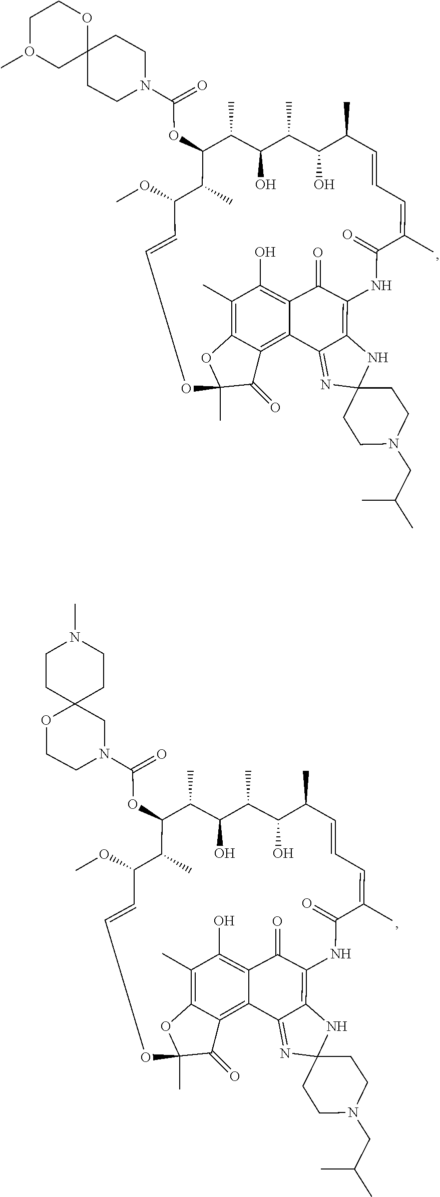

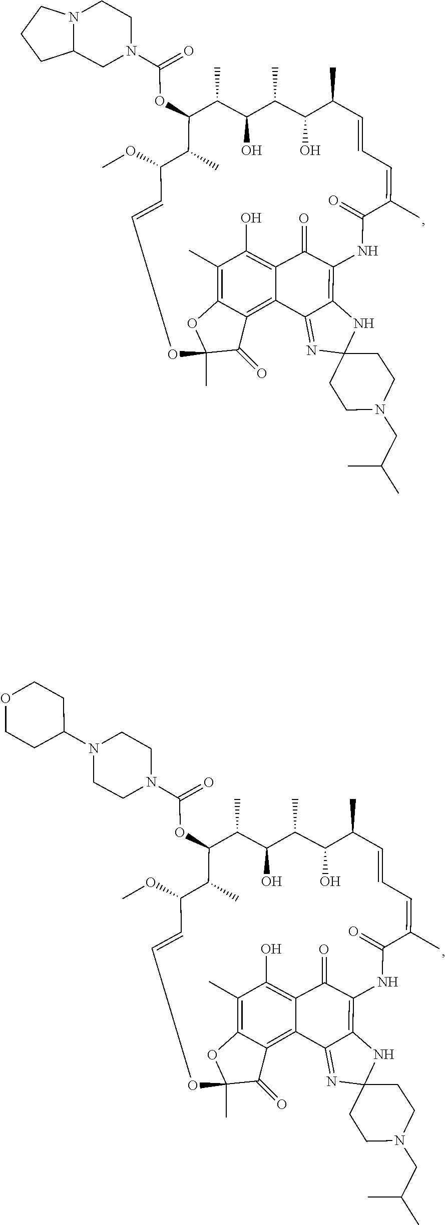 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN3CCCC3C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCOCC3)CC2)[C@@H]1C)C4=O]

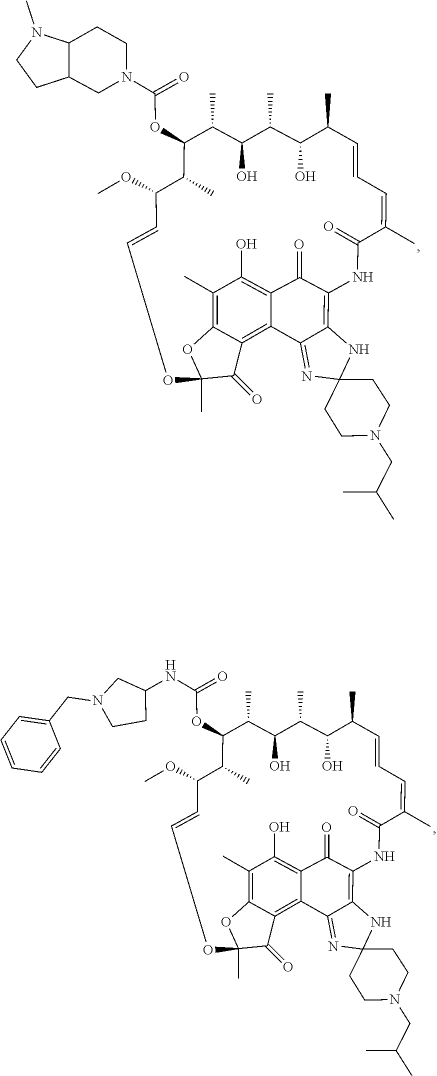 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3C(CCN3C)C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)C2)[C@@H]1C)C4=O]

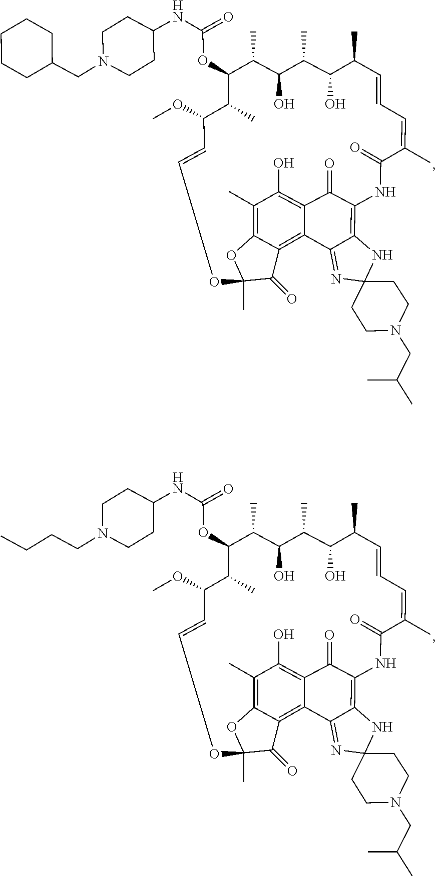

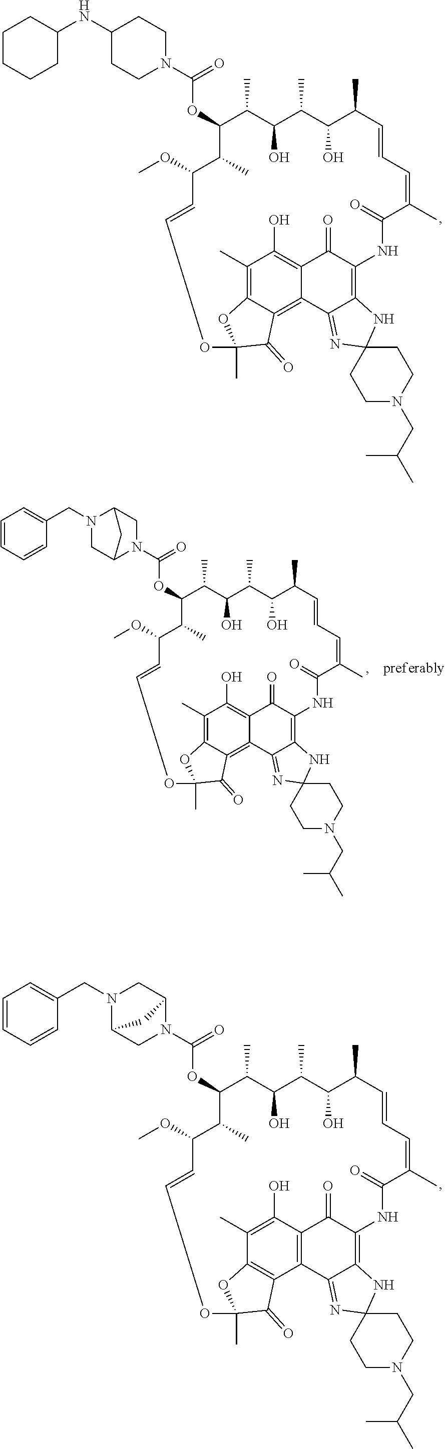

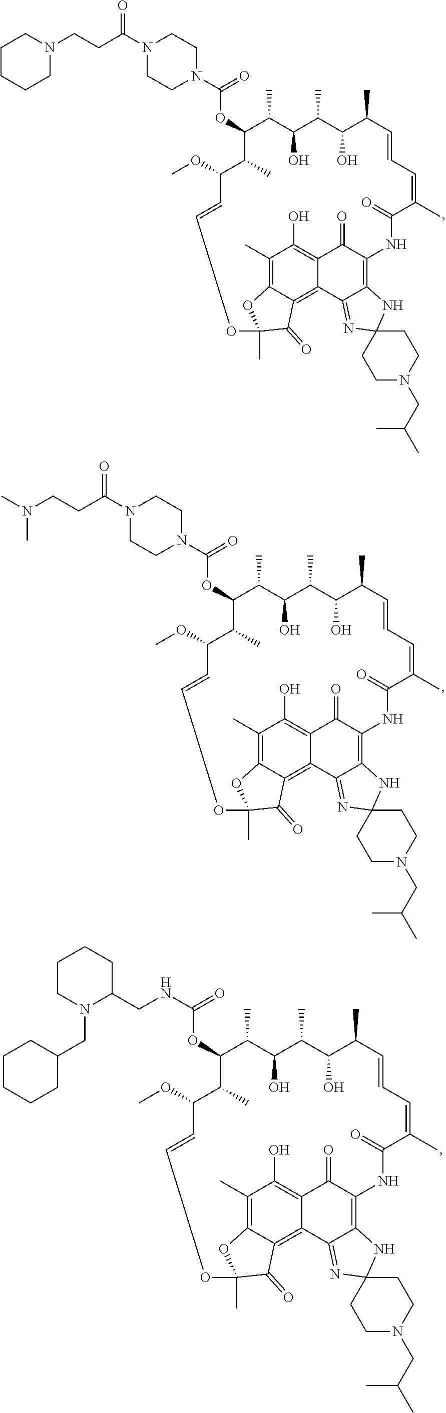

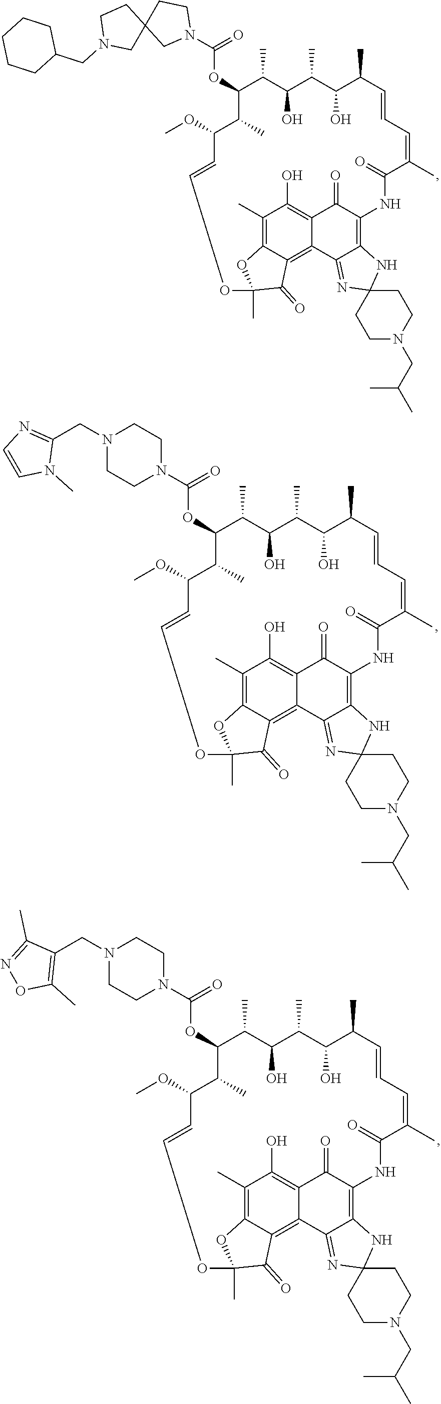

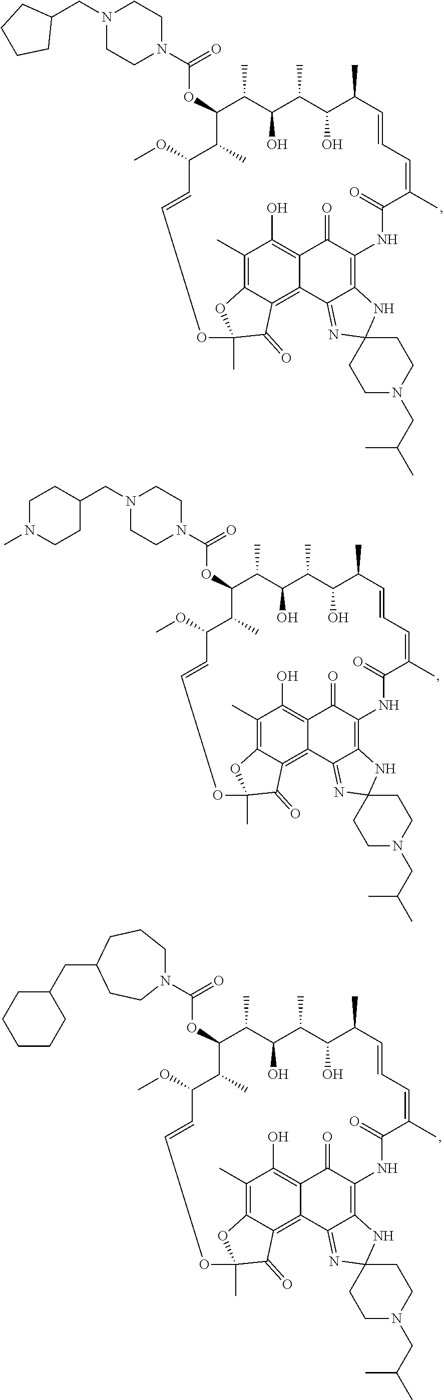

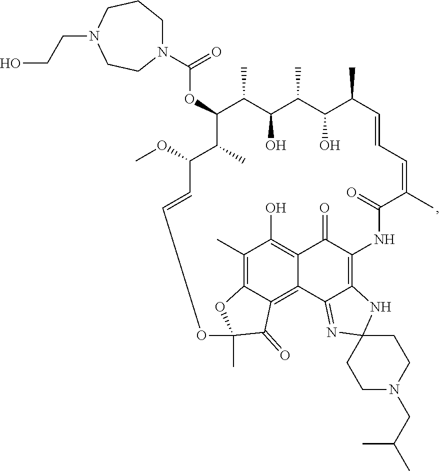 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(CCO)CC2)[C@@H]1C)C4=O]

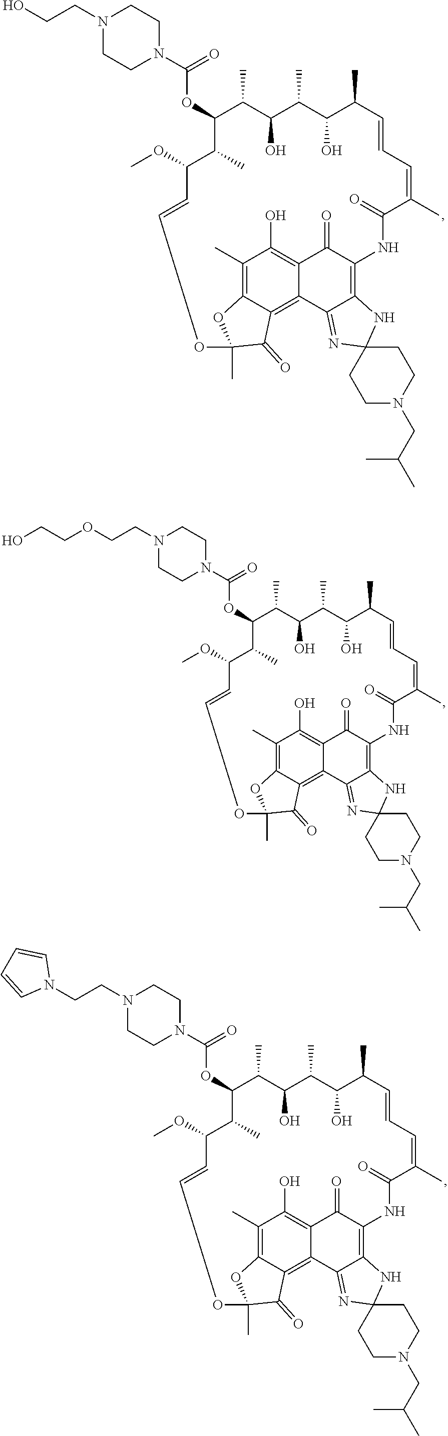

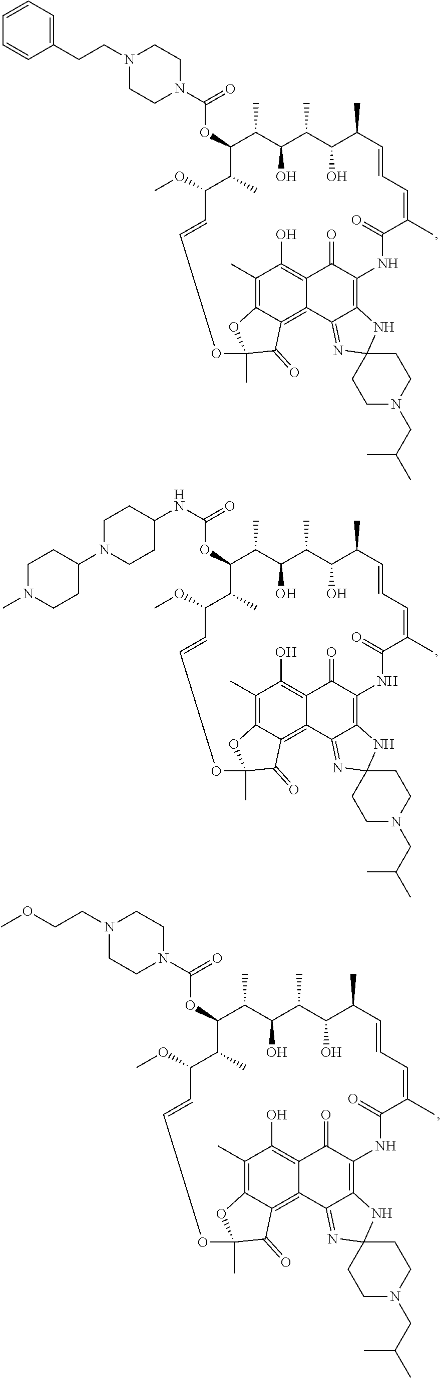

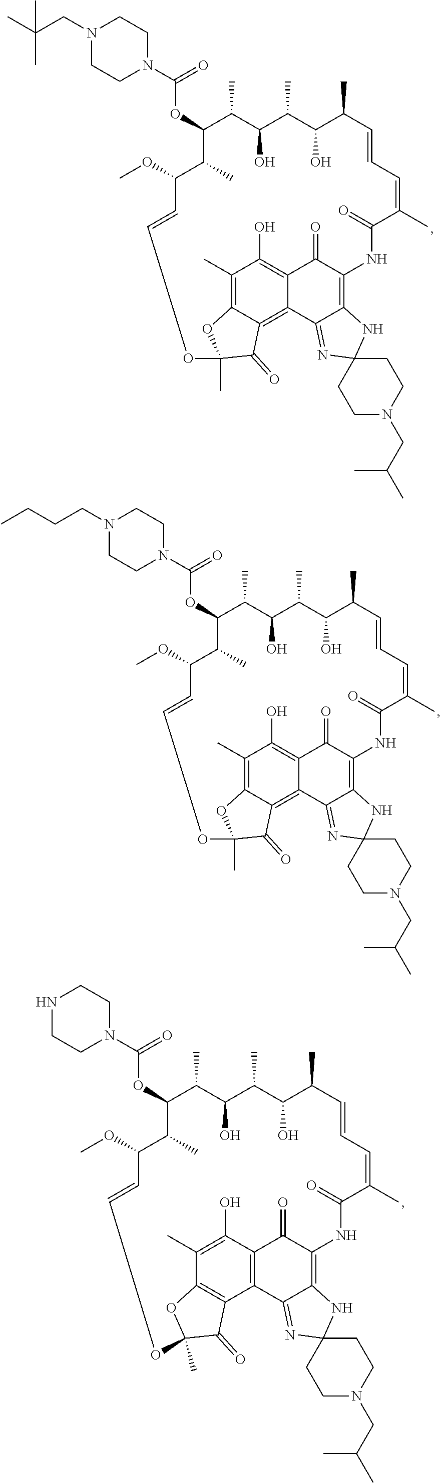

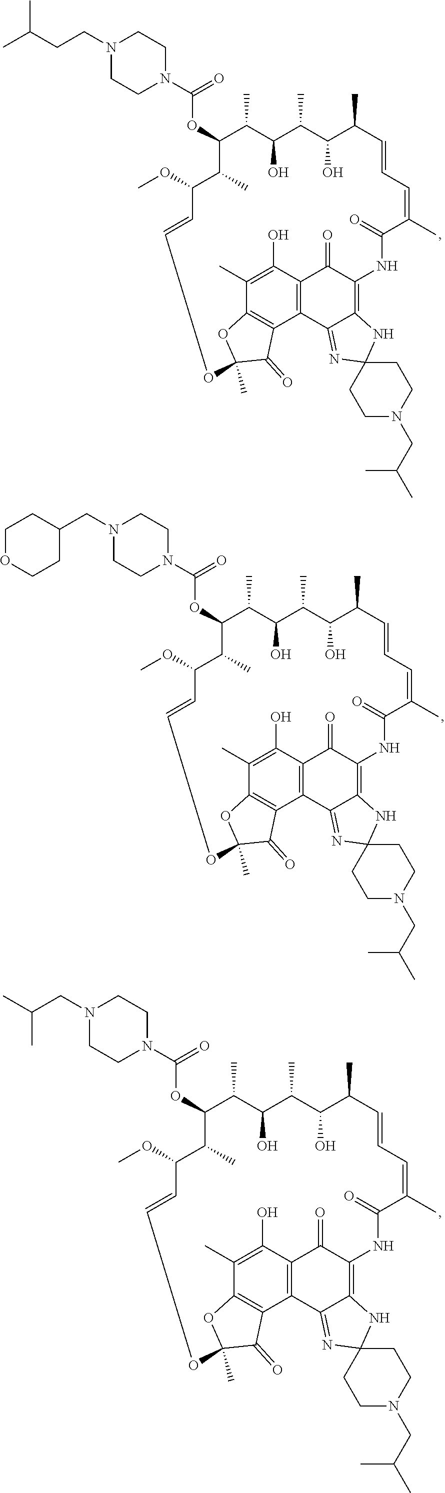

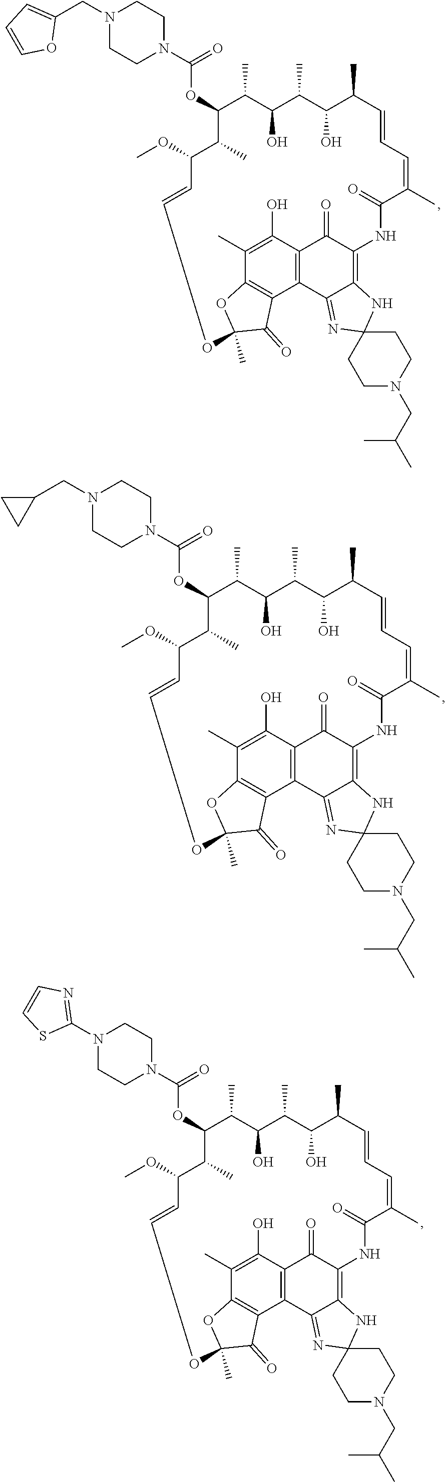

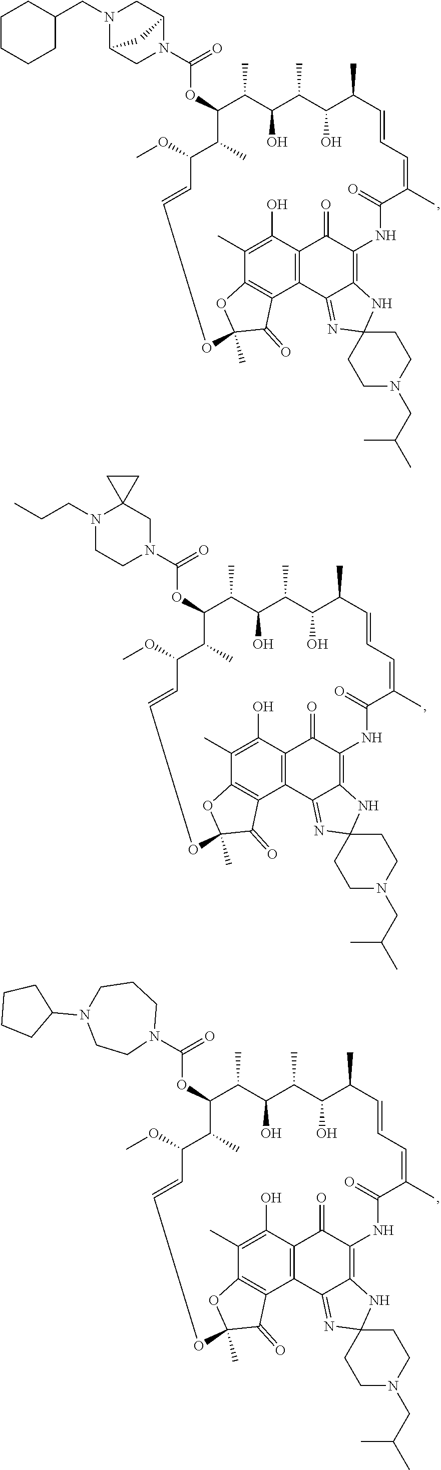

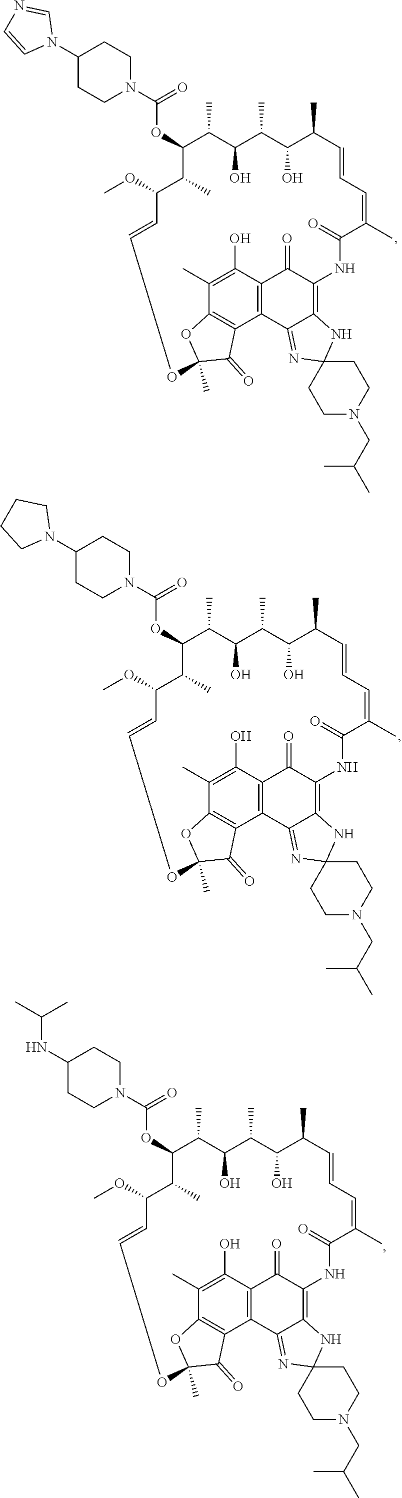

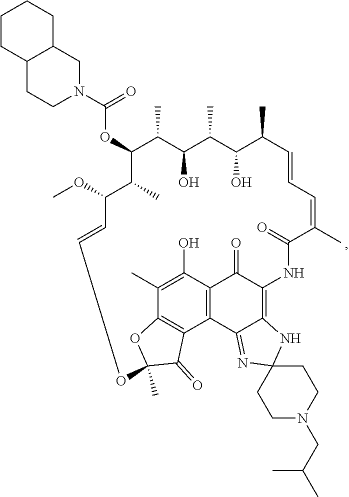 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3CCCCC3C2)[C@@H]1C)C4=O]

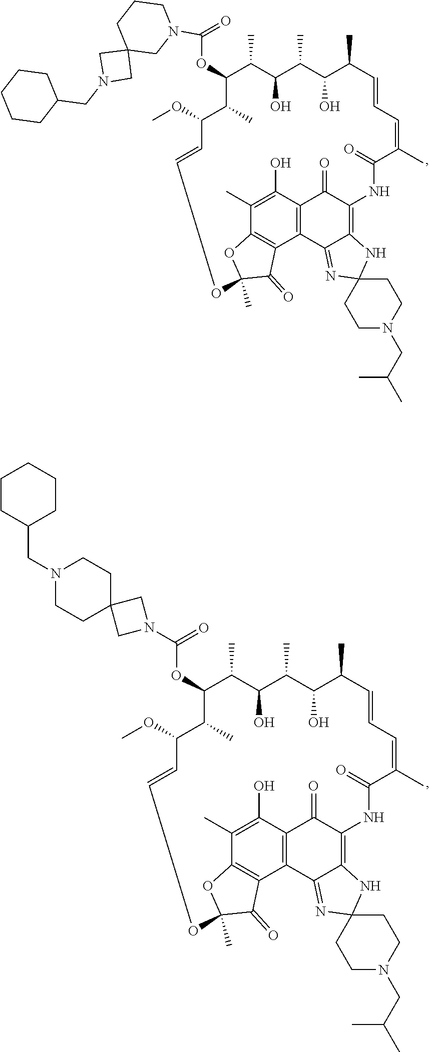 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CN(CC5CCCCC5)C3)C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3(CCN(CC5CCCCC5)CC3)C2)[C@@H]1C)C4=O]

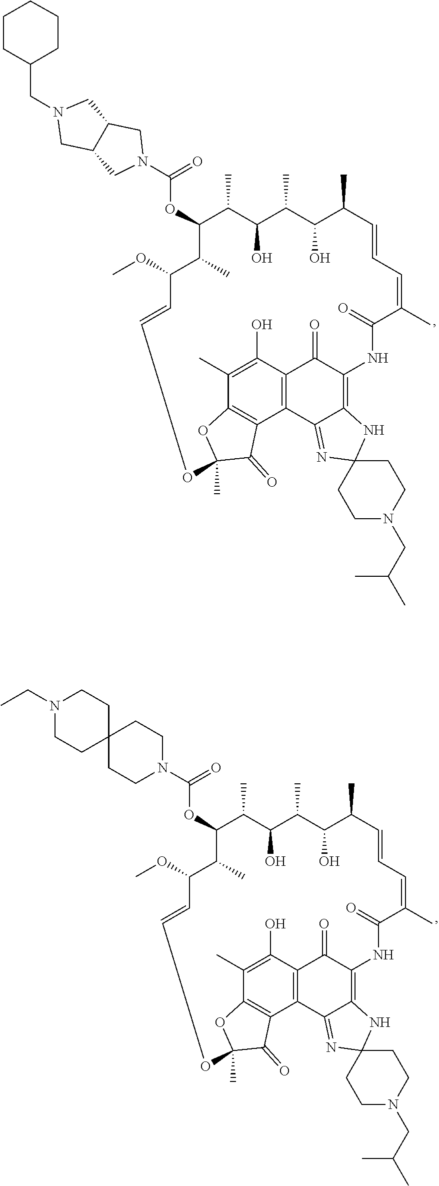

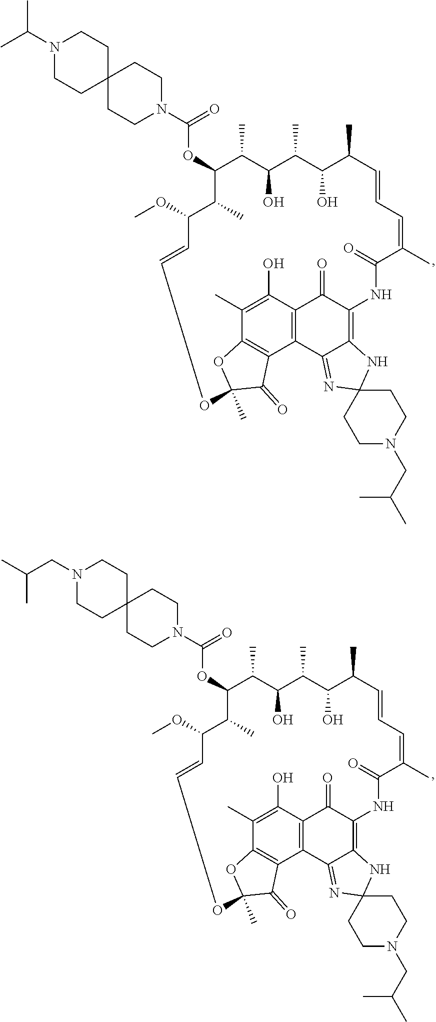

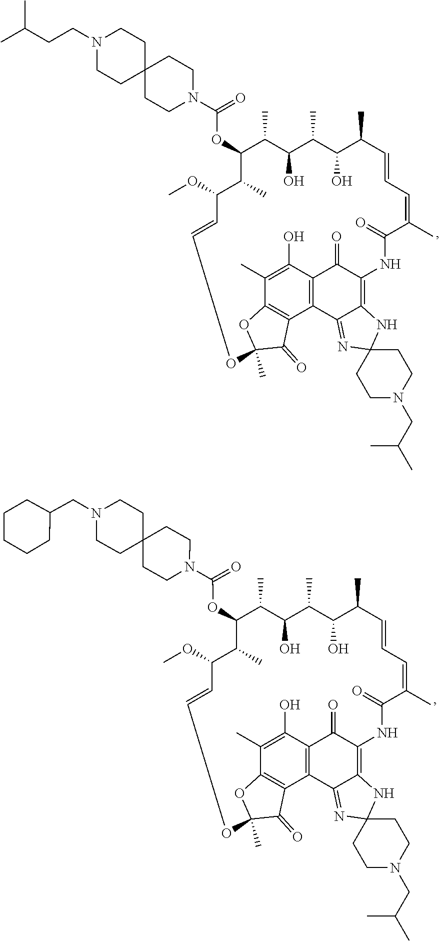 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CCC(C)C)CC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC5CCCCC5)CC3)CC2)[C@@H]1C)C4=O]

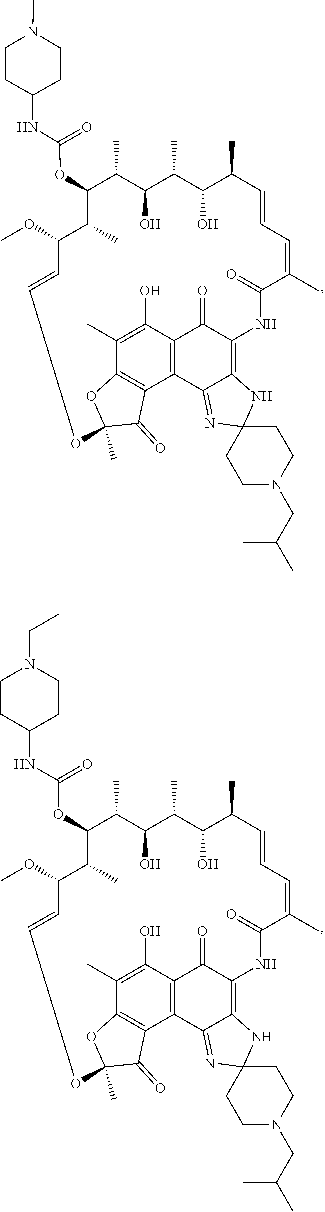

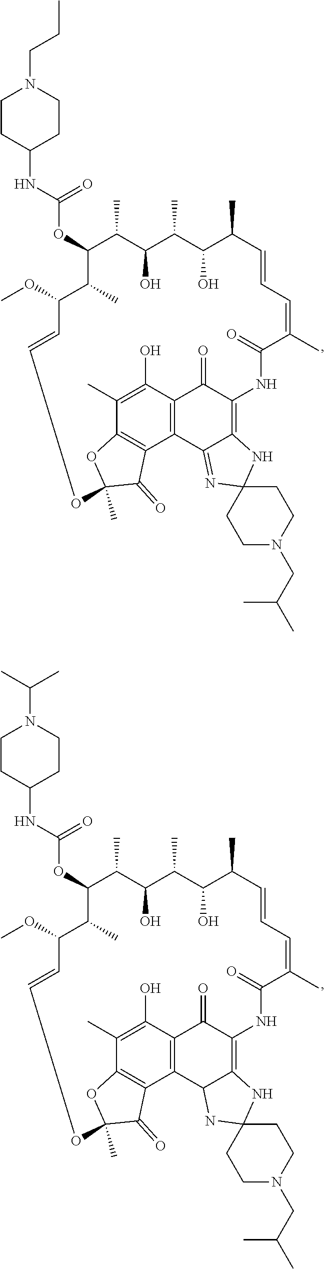

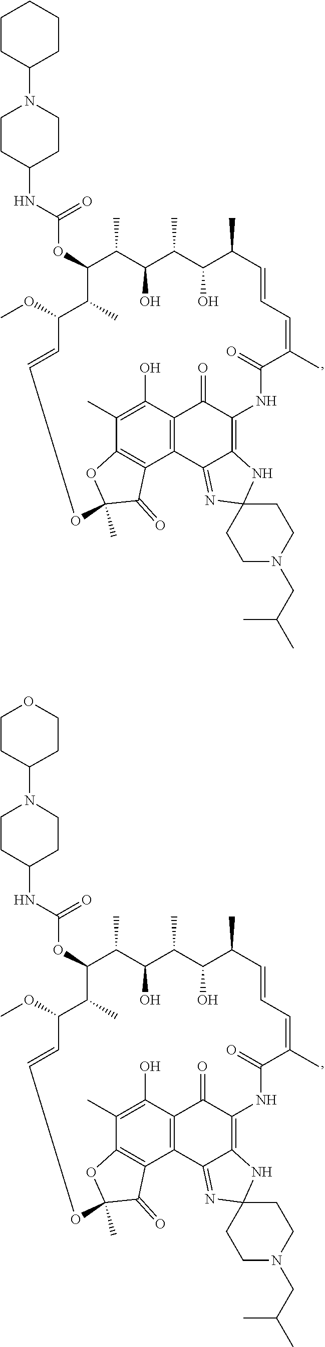

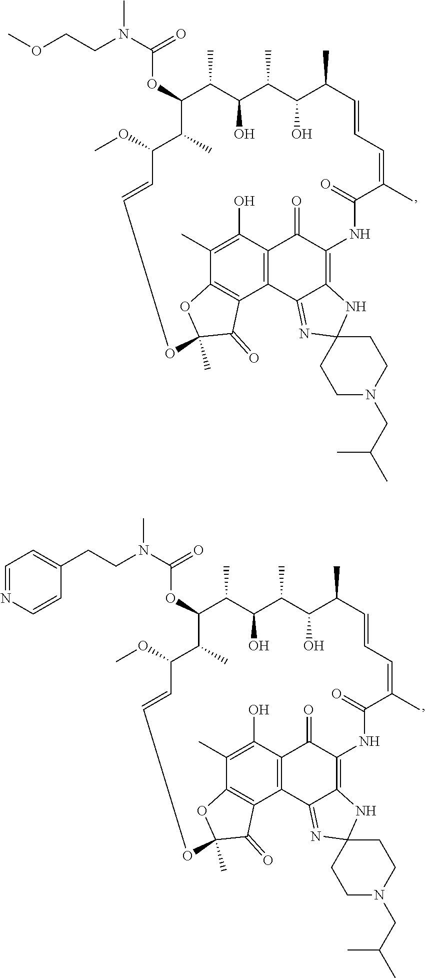

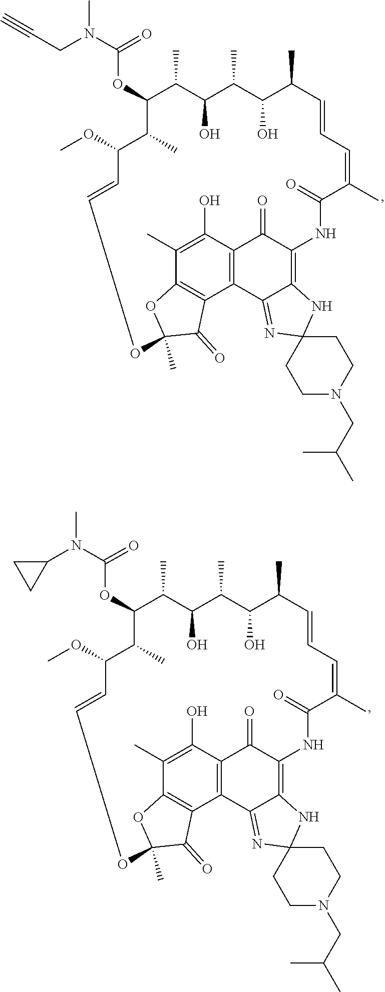

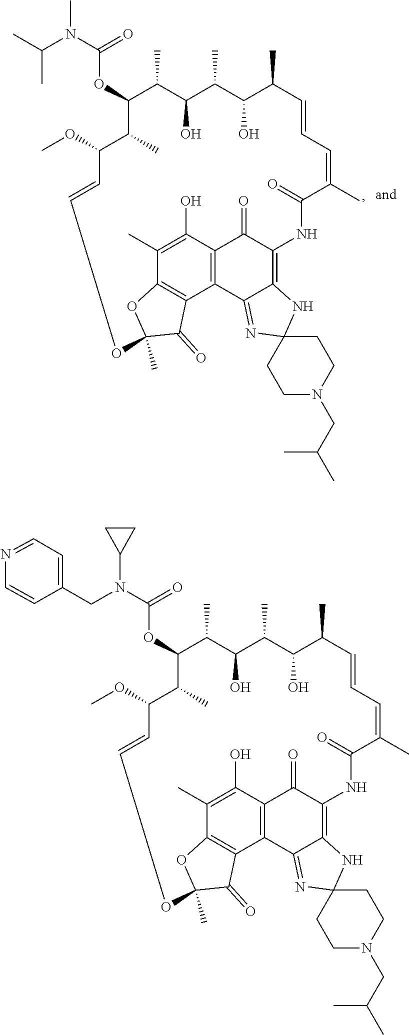 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(C)C(C)C)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N(Cc2ccncc2)C2CC2)[C@@H]1C)C4=O]

11. The compound according to claim 1, wherein the compound is selected from the group consisting of: 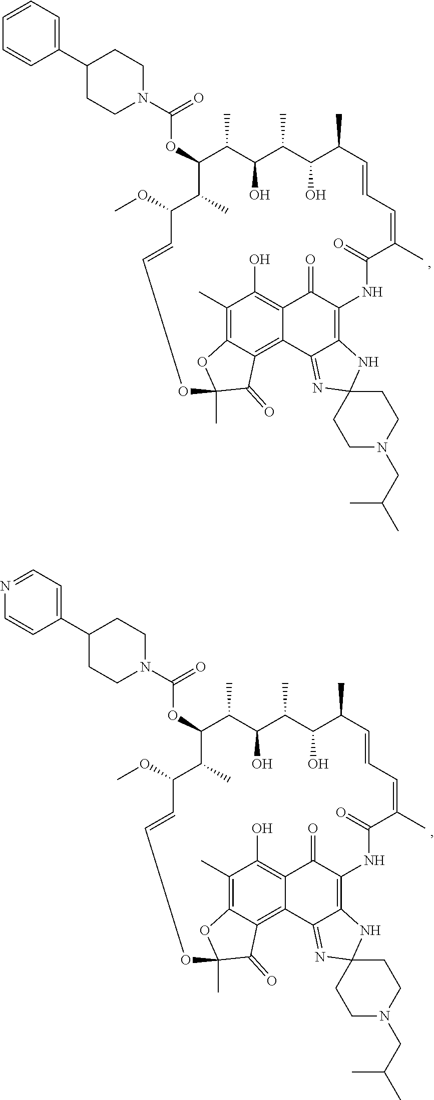 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccncc3)CC2)[C@@H]1C)C4=O] 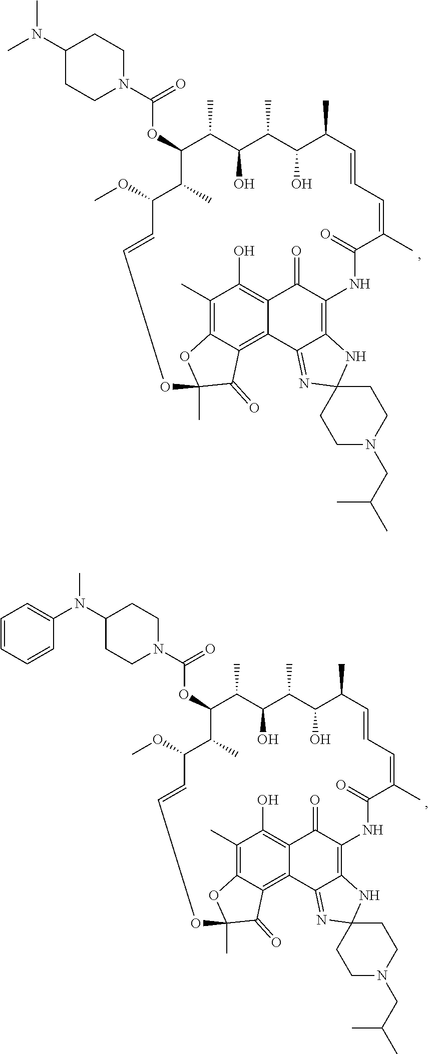 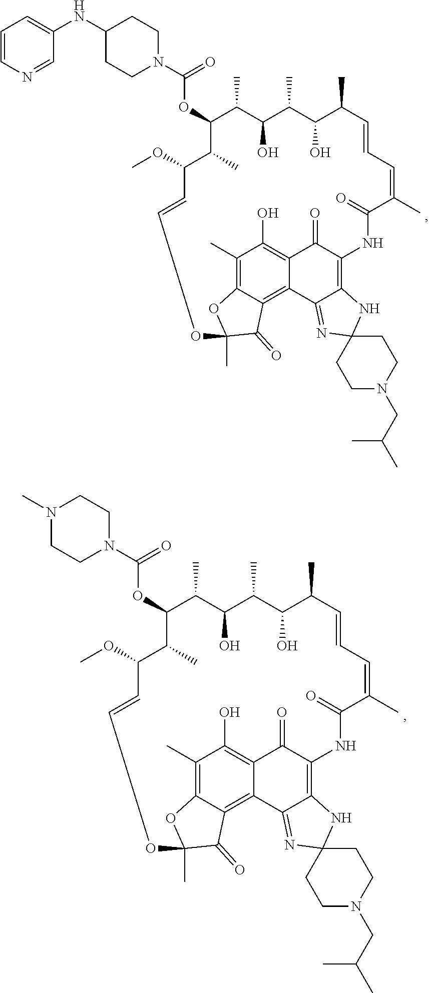 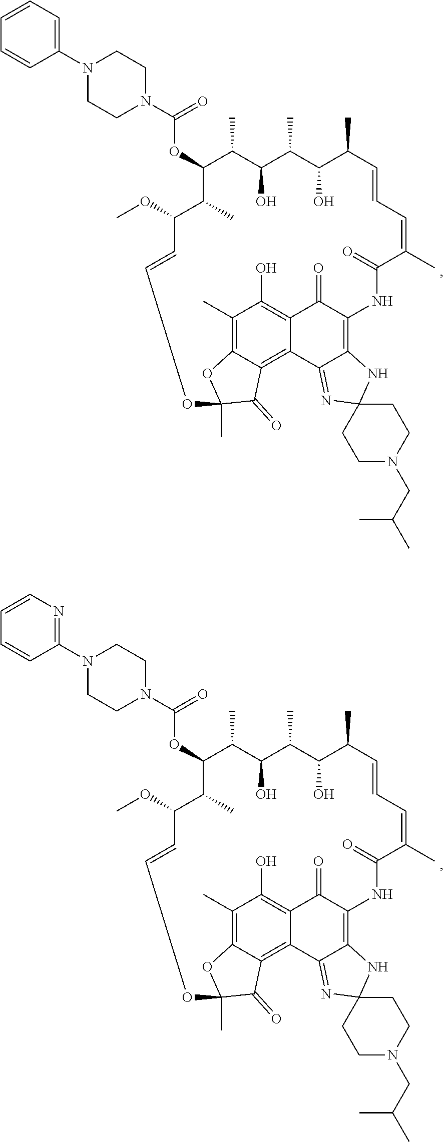 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccn3)CC2)[C@@H]1C)C4=O] 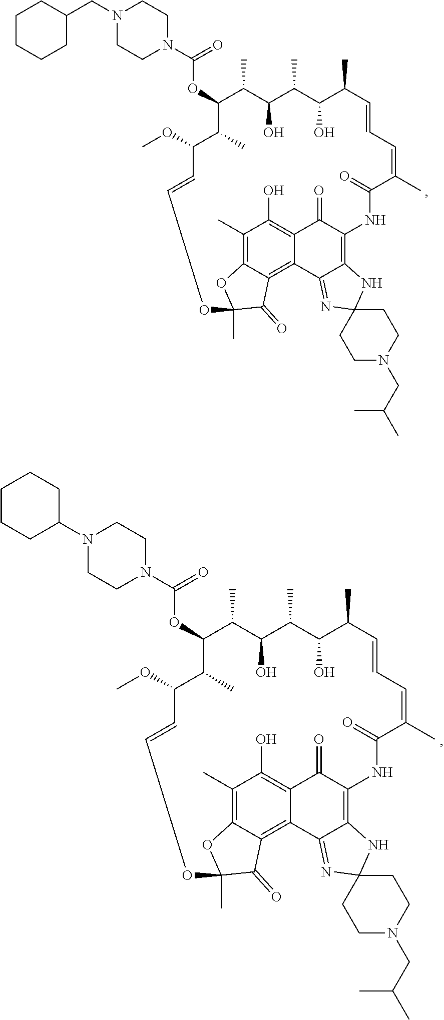 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCCC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCCCC3)CC2)[C@@H]1C)C4=O] 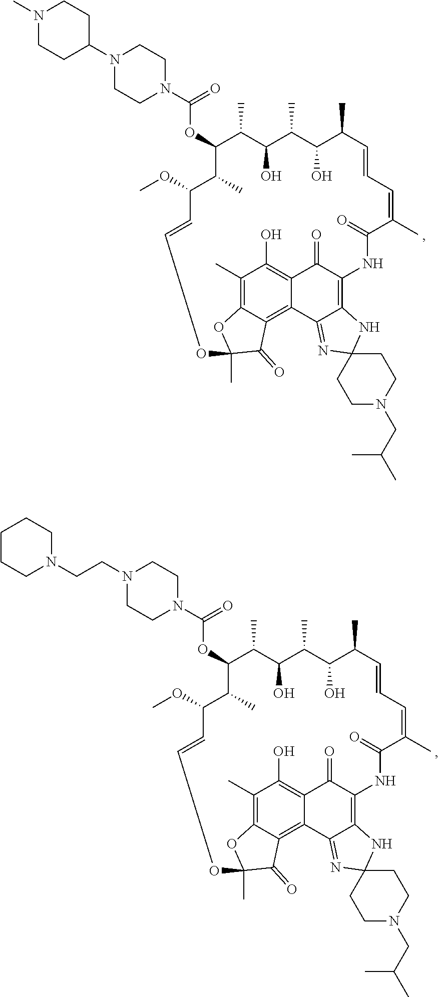 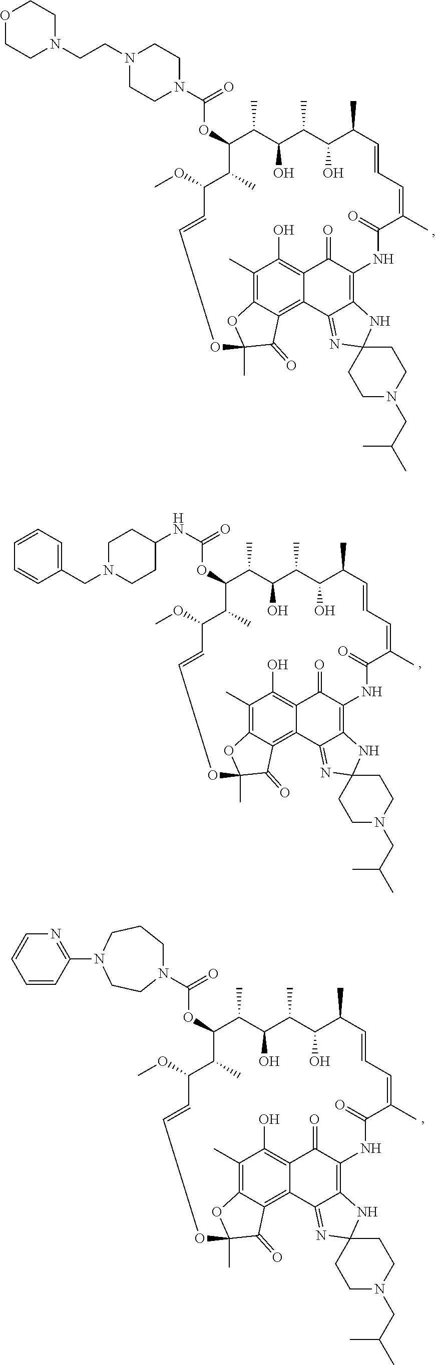 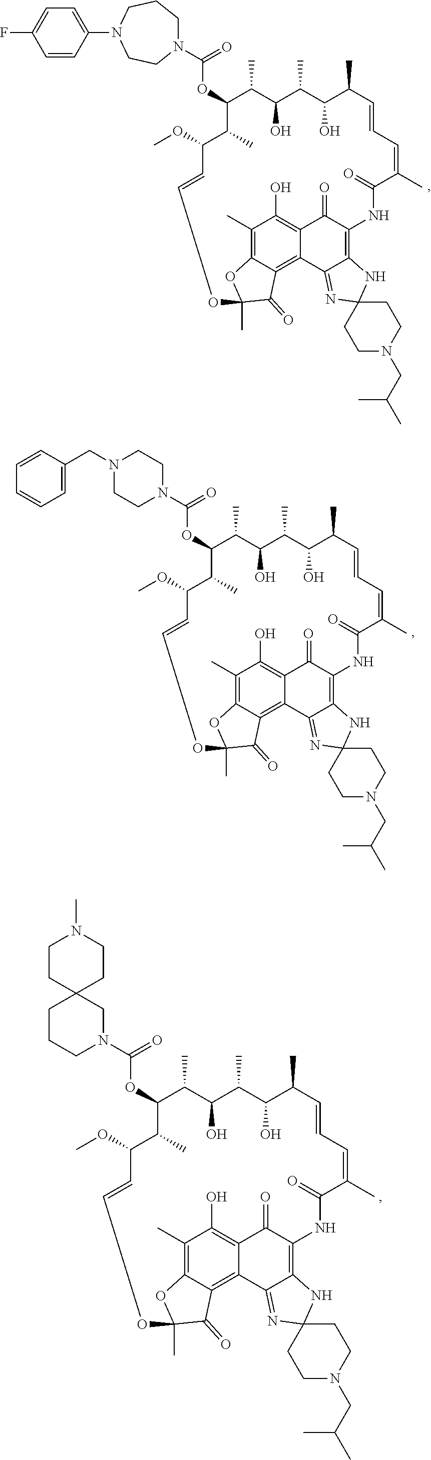 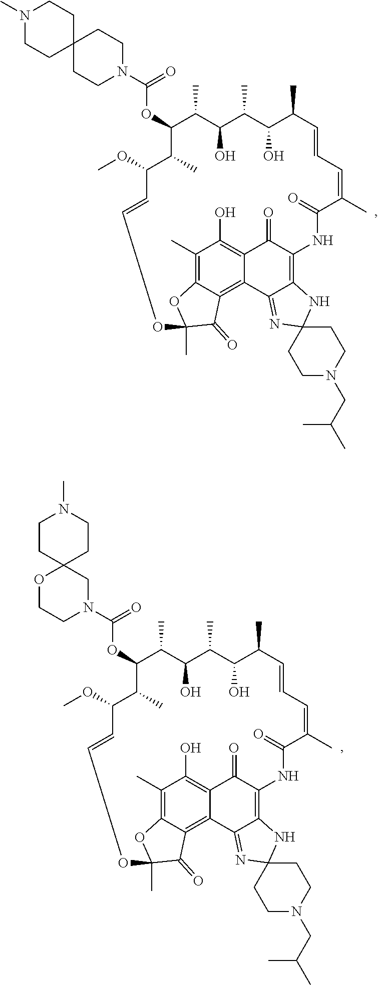 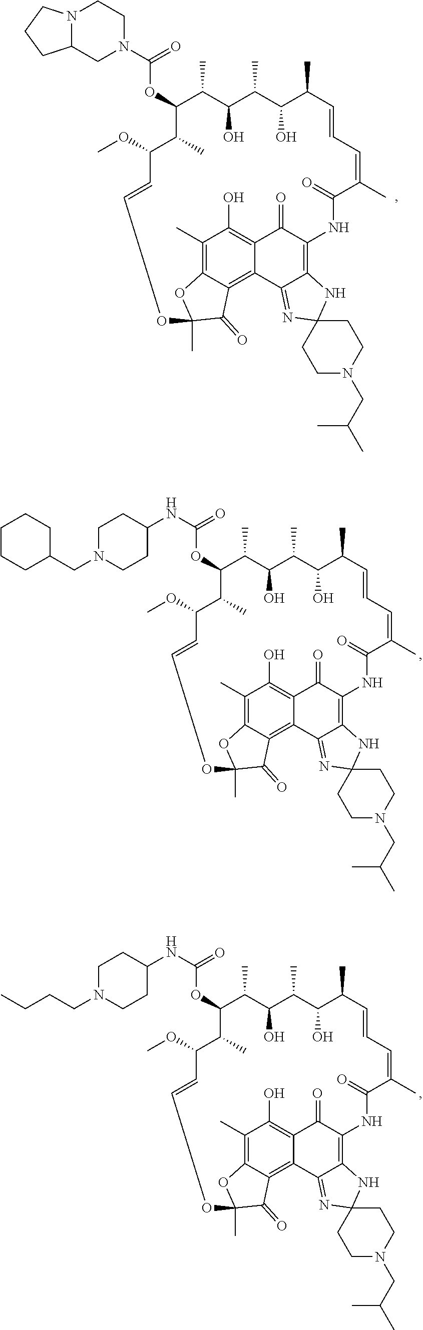 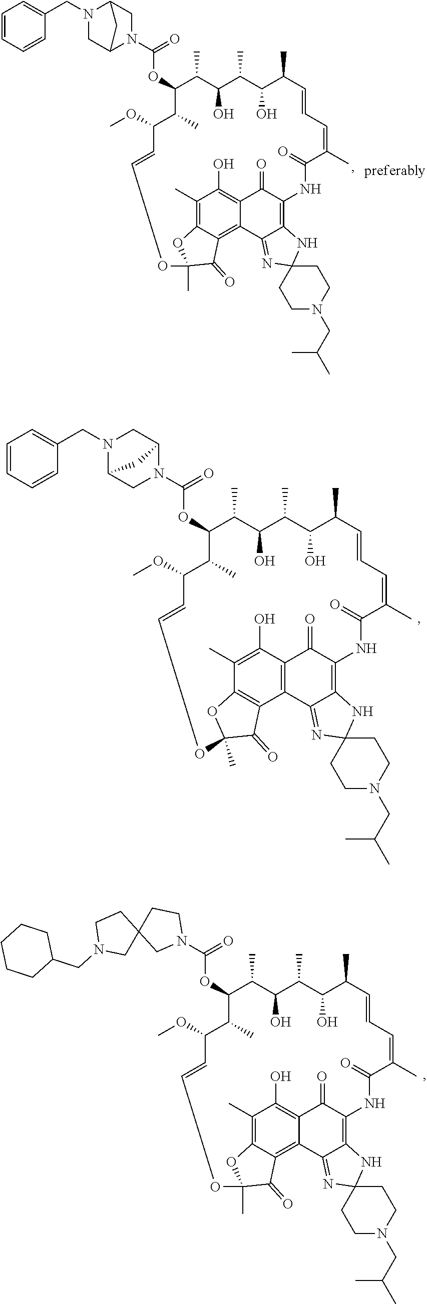 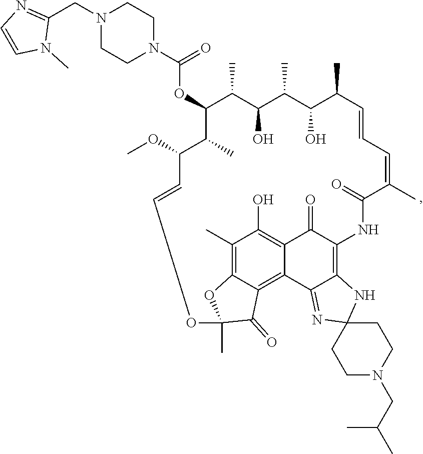 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3nccn3C)CC2)[C@@H]1C)C4=O] 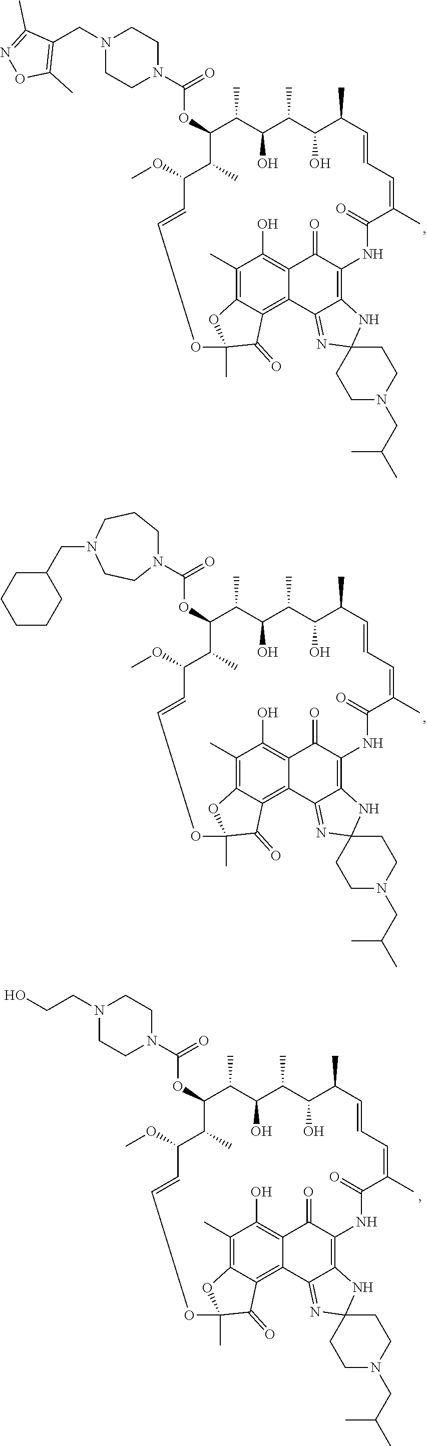 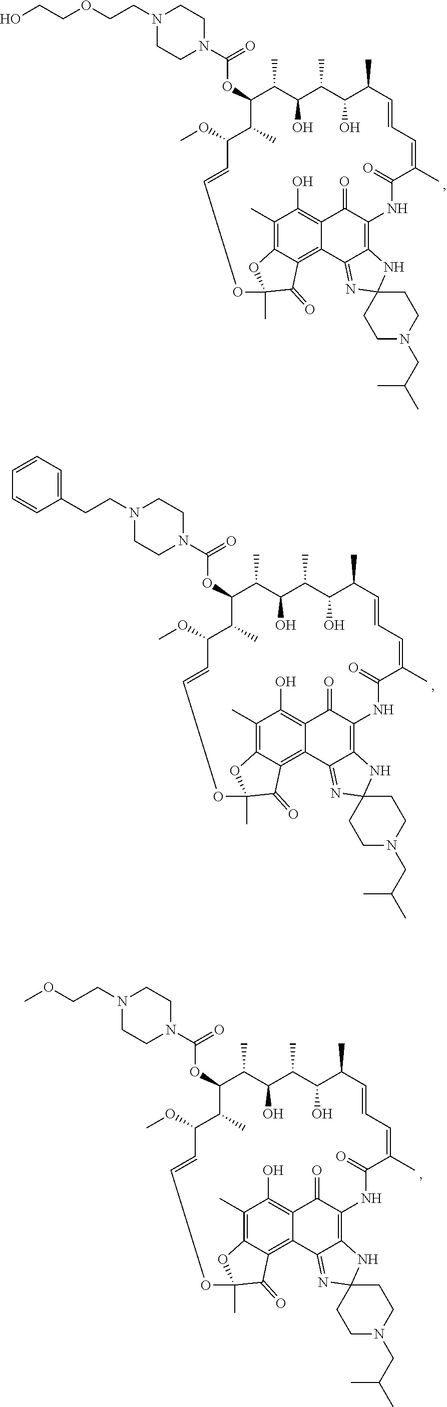 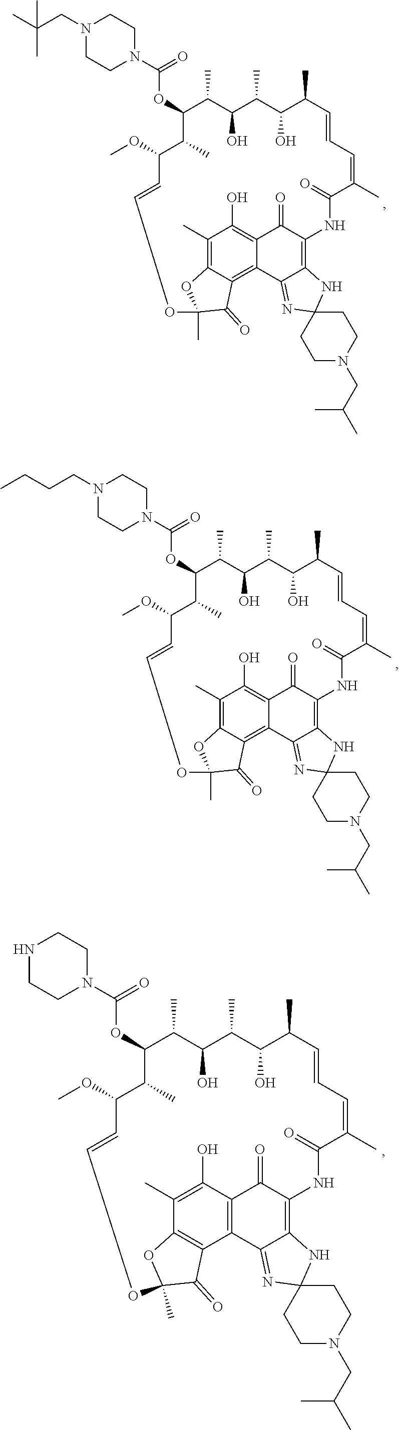 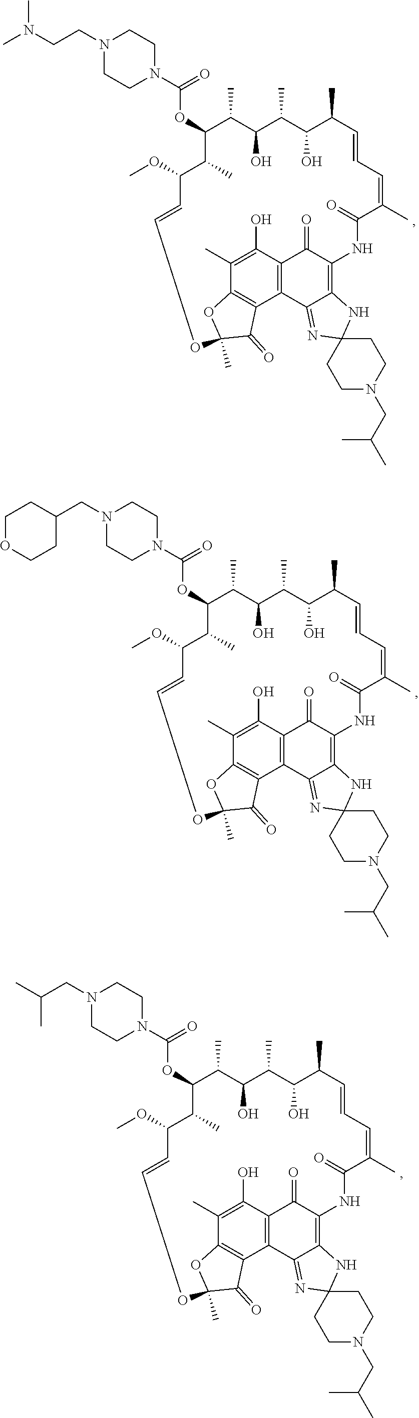 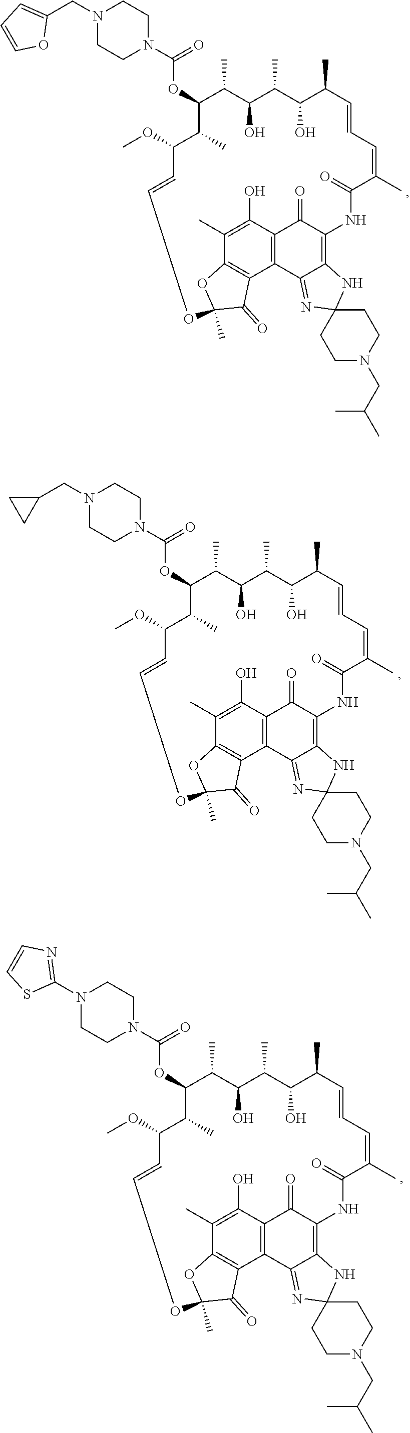 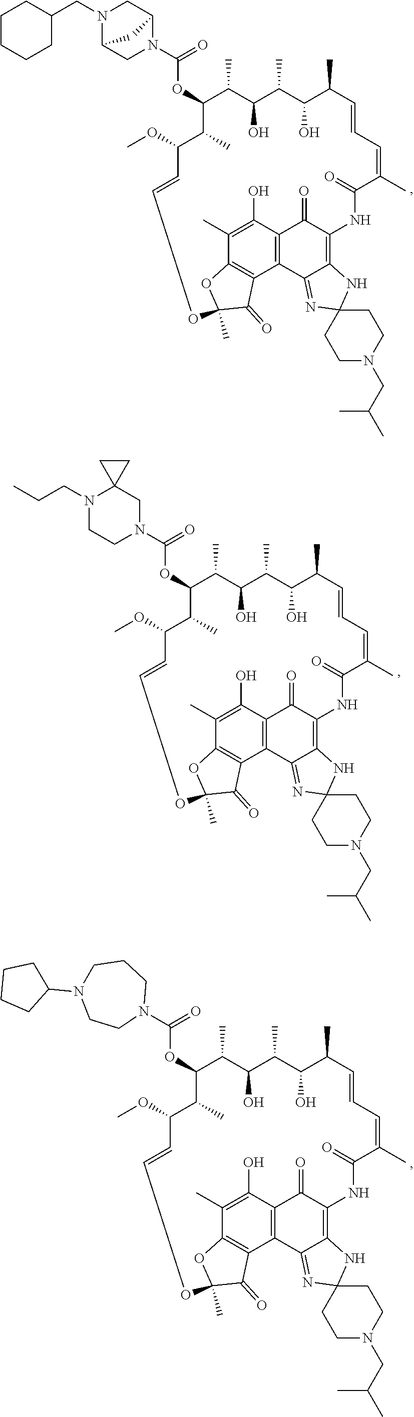 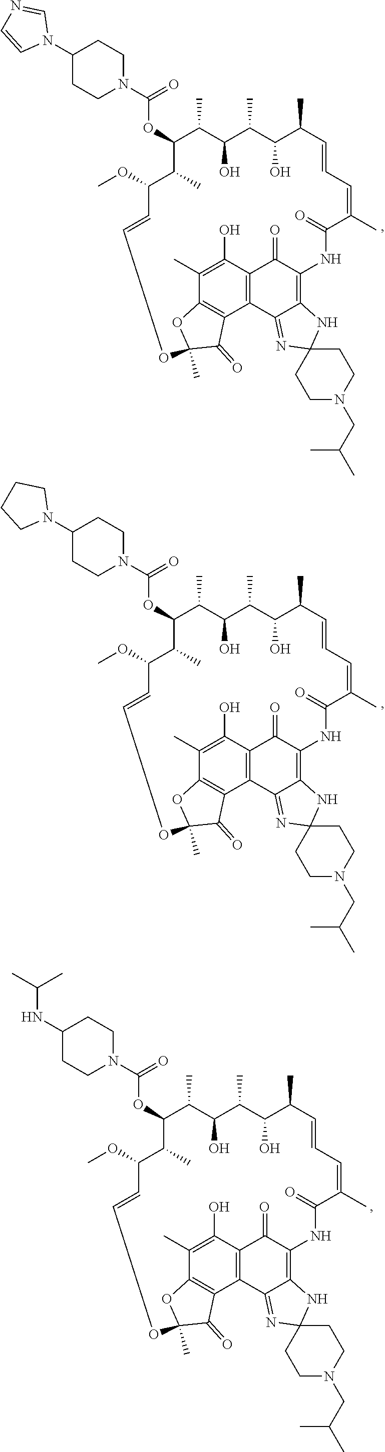 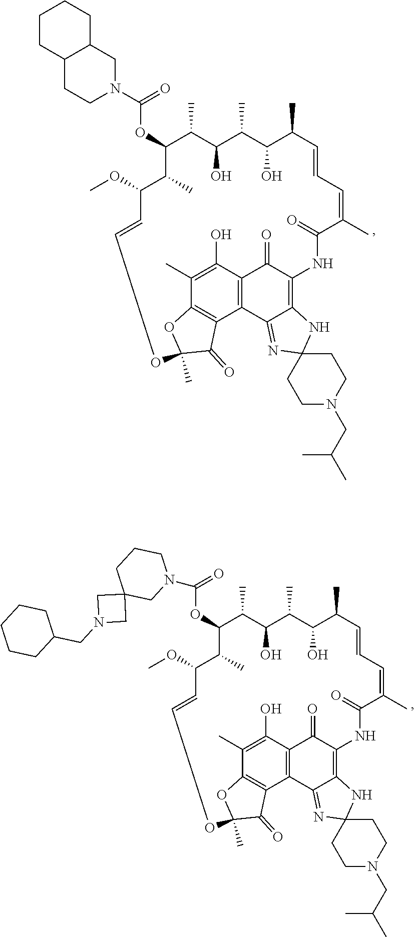 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3CCCCC3C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CN(CC5CCCCC5)C3)C2)[C@@H]1C)C4=O] 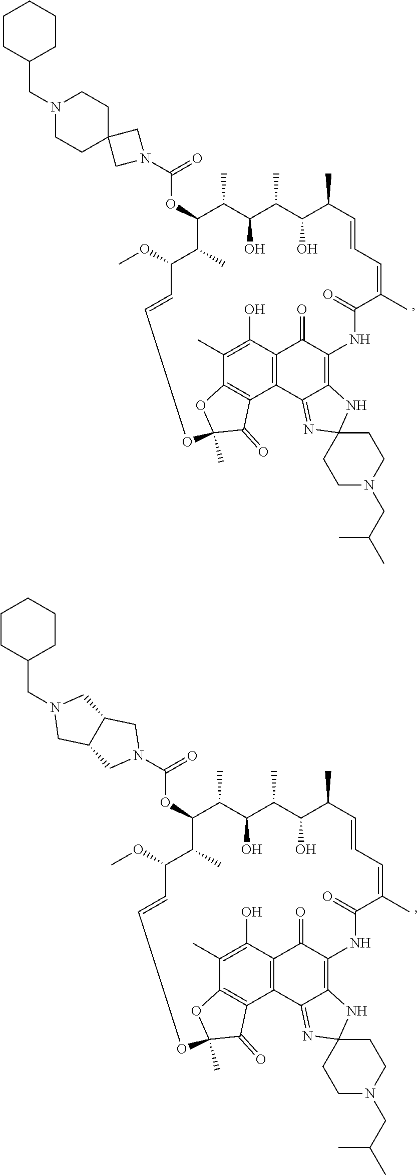 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CC3(CCN(CC5CCCCC5)CC3)C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2C[C@H]3CN(CC5CCCCC5)C[C@H]3C2)[C@@H]1C)C4=O] 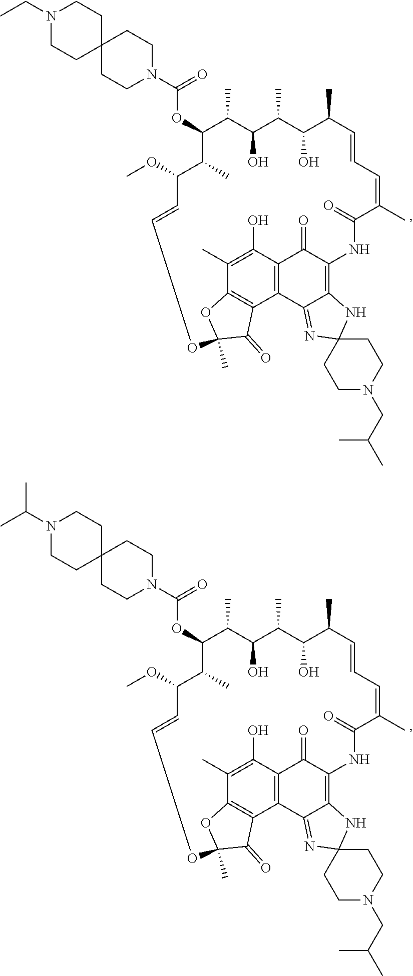 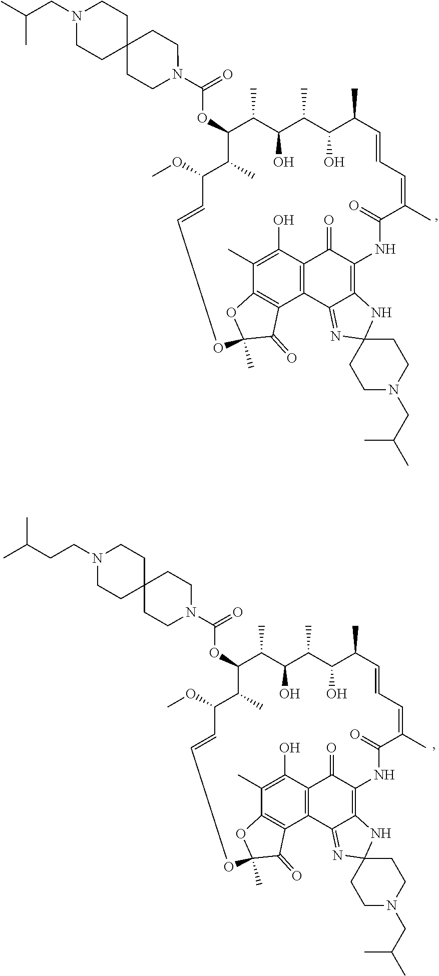 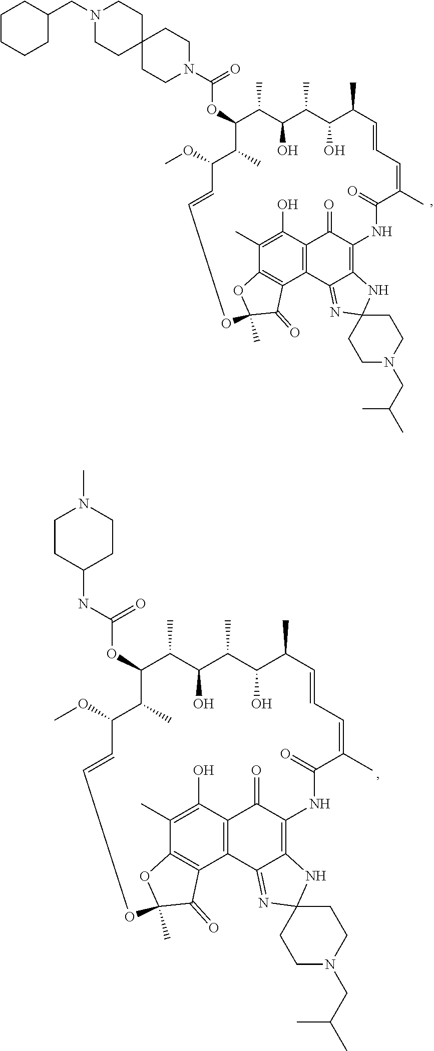 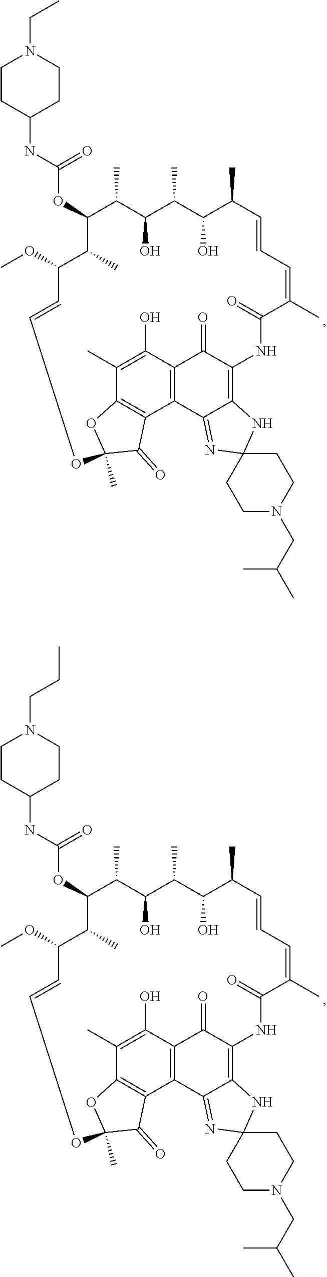 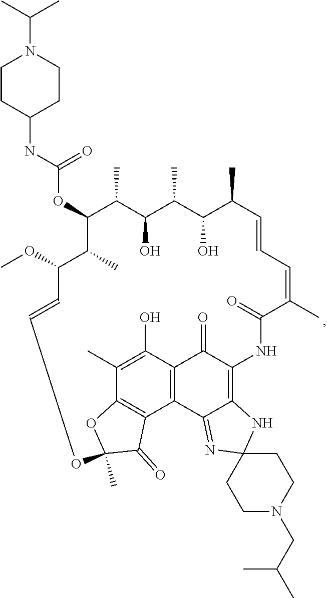 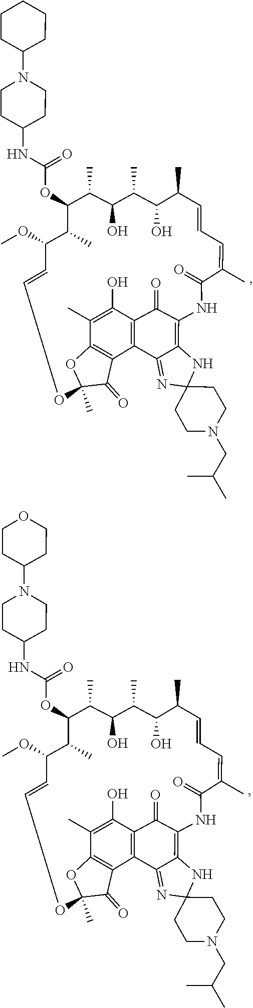 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(C3CCCCC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(C3CCOCC3)CC2)[C@@H]1C)C4=O] 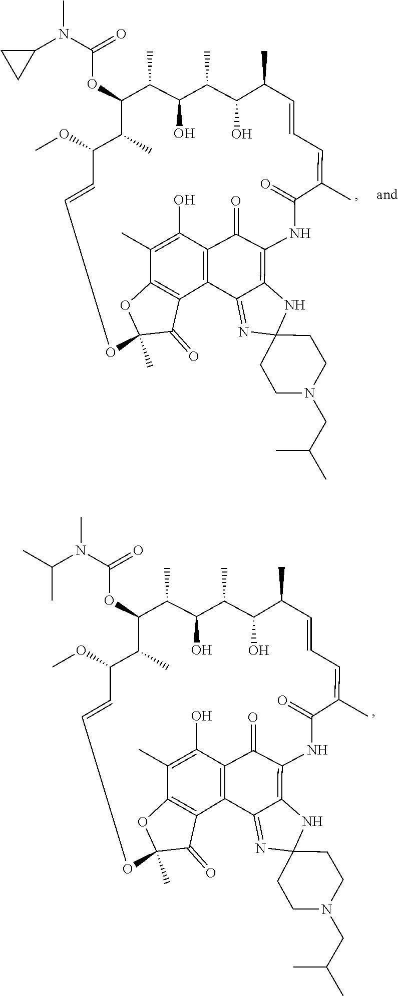
or a pharmaceutically acceptable salt thereof.

12. The compound according to claim 1, wherein the compound is selected from the group consisting of: 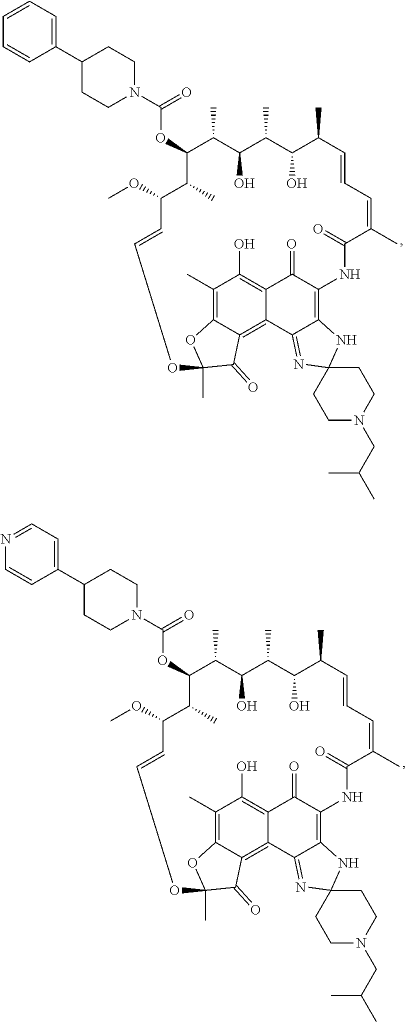 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC(c3ccncc3)CC2)[C@@H]1C)C4=O] 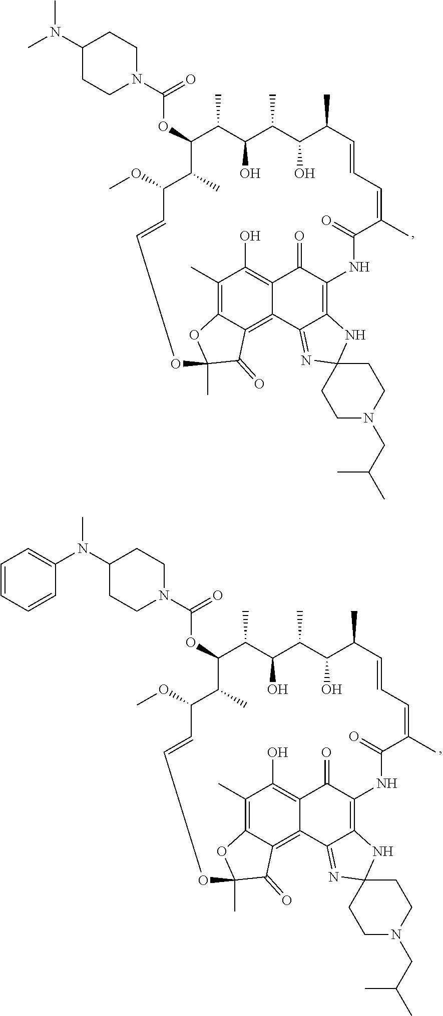 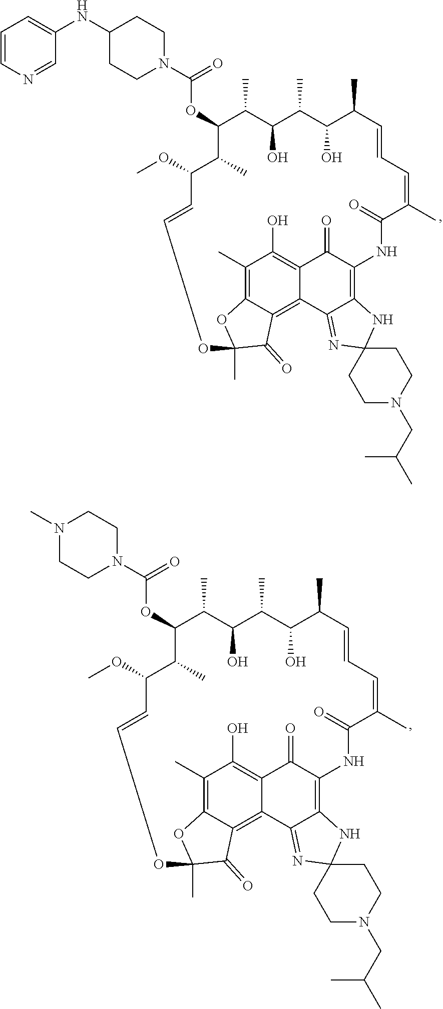 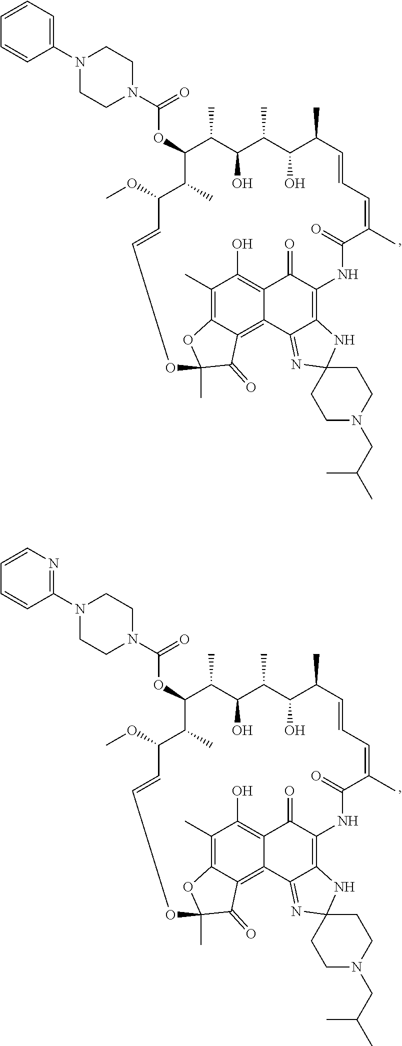 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(c3ccccn3)CC2)[C@@H]1C)C4=O] 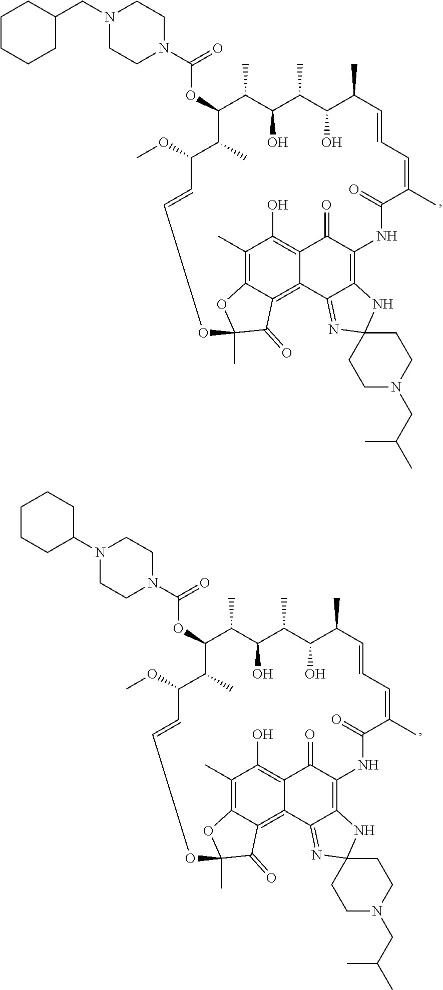 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CC3CCCCC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(C3CCCCC3)CC2)[C@@H]1C)C4=O] 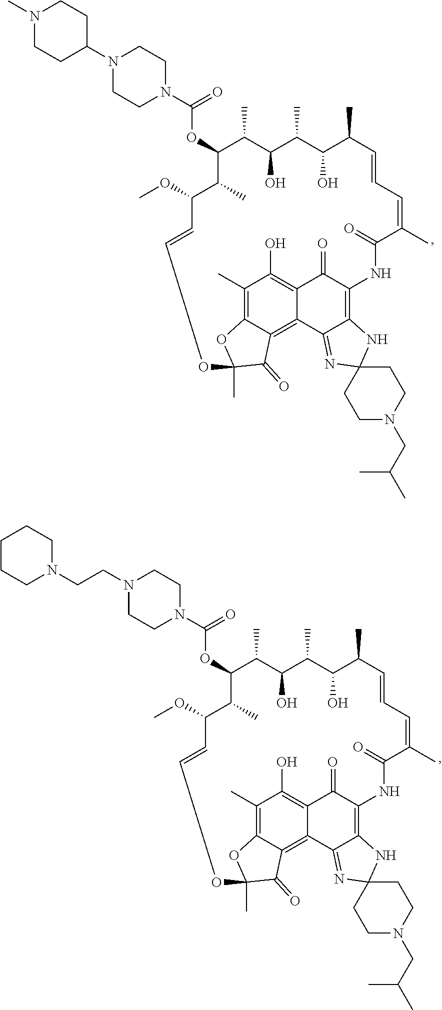 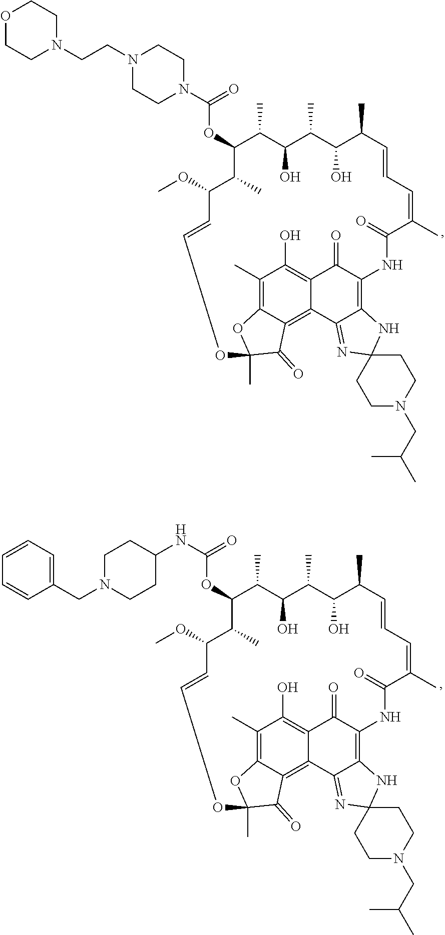 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(CCN3CCOCC3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(Cc3ccccc3)CC2)[C@@H]1C)C4=O] 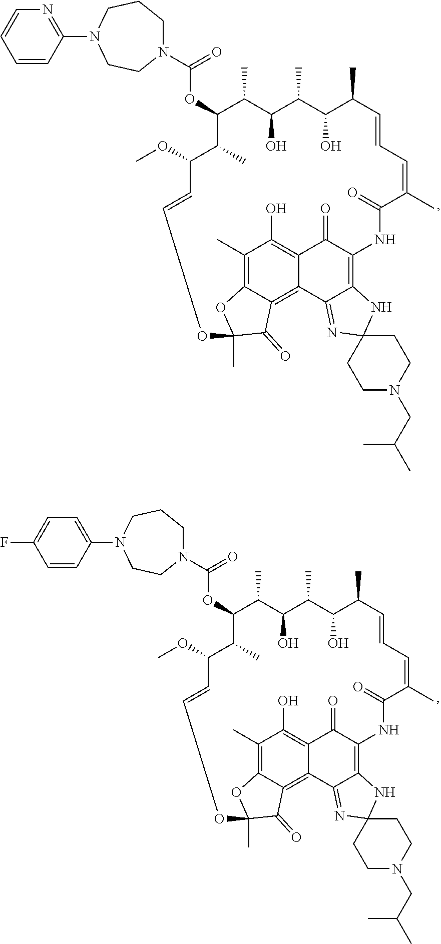 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccccn3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCN(c3ccc(F)cc3)CC2)[C@@H]1C)C4=O] 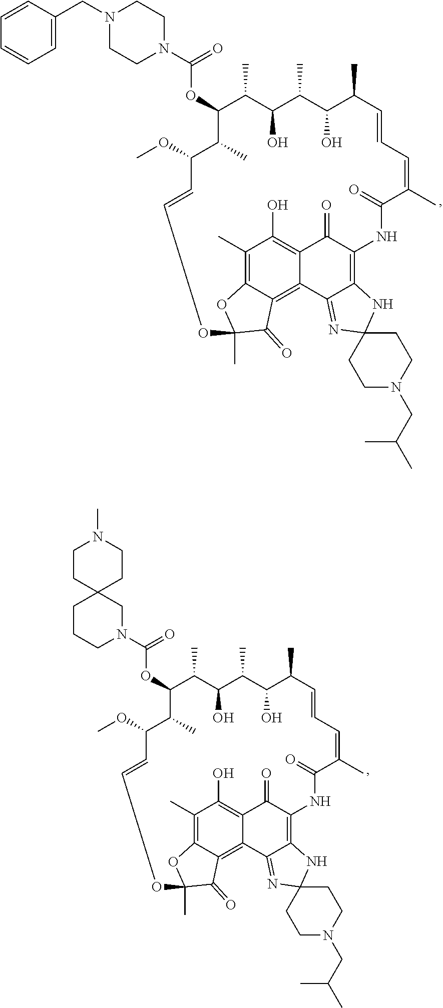 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3ccccc3)CC2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCCC3(CCN(C)CC3)C2)[C@@H]1C)C4=O] 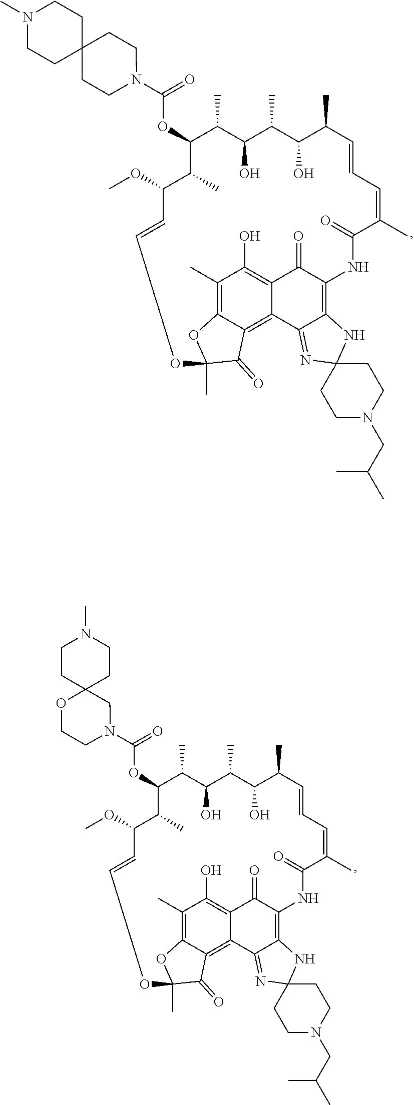 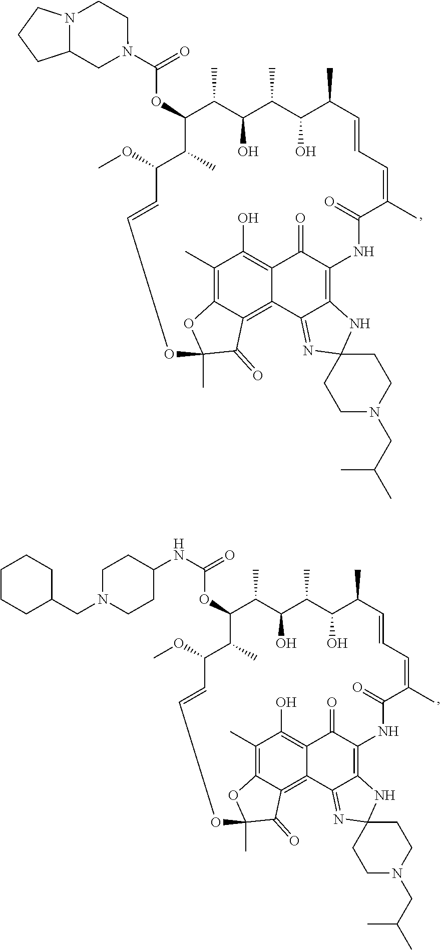 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN3CCCC3C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)NC2CCN(CC3CCCCC3)CC2)[C@@H]1C)C4=O] 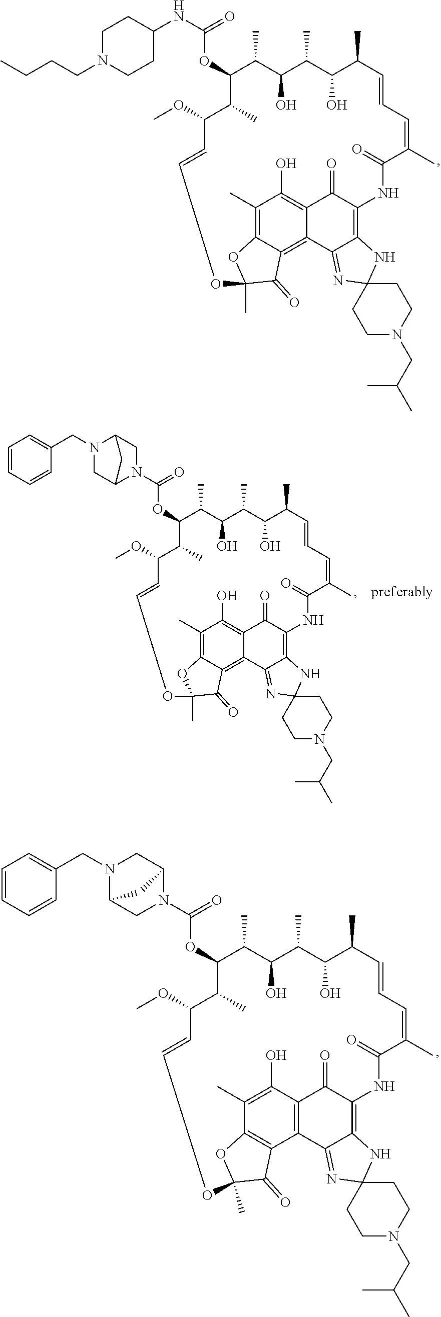 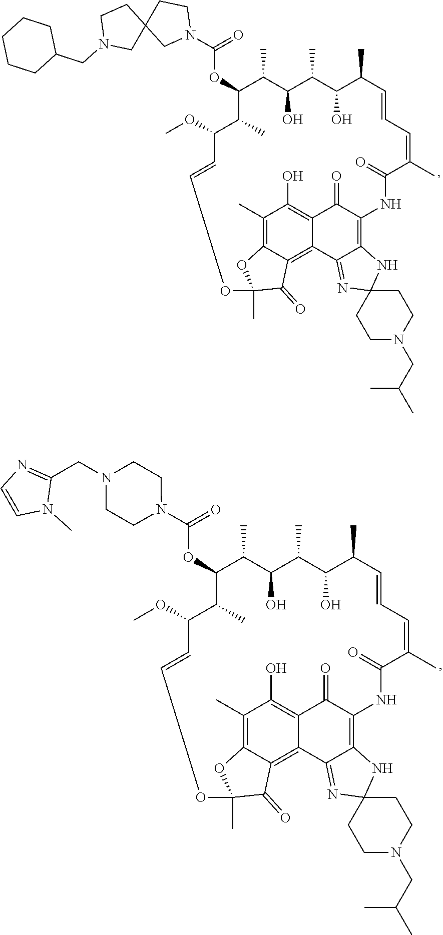 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCC3(CCN(CC5CCCCC5)C3)C2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)N2CCN(Cc3nccn3C)CC2)[C@@H]1C)C4=O] 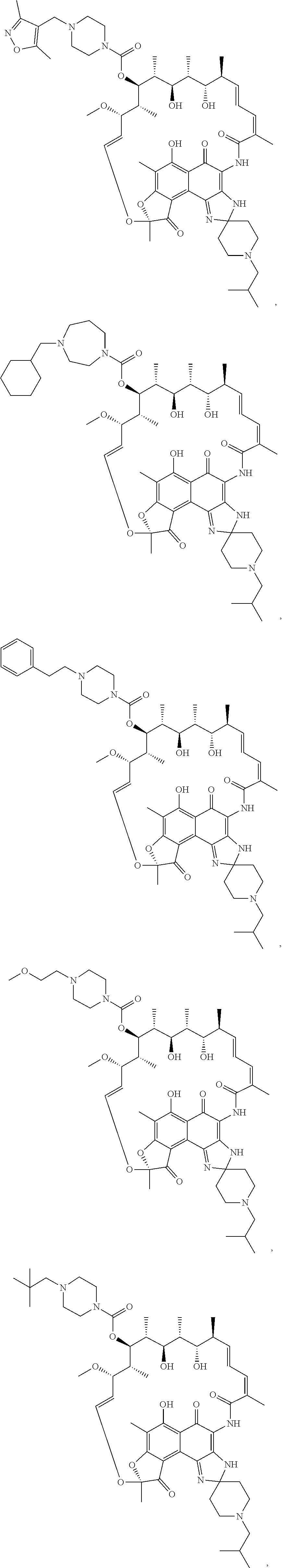 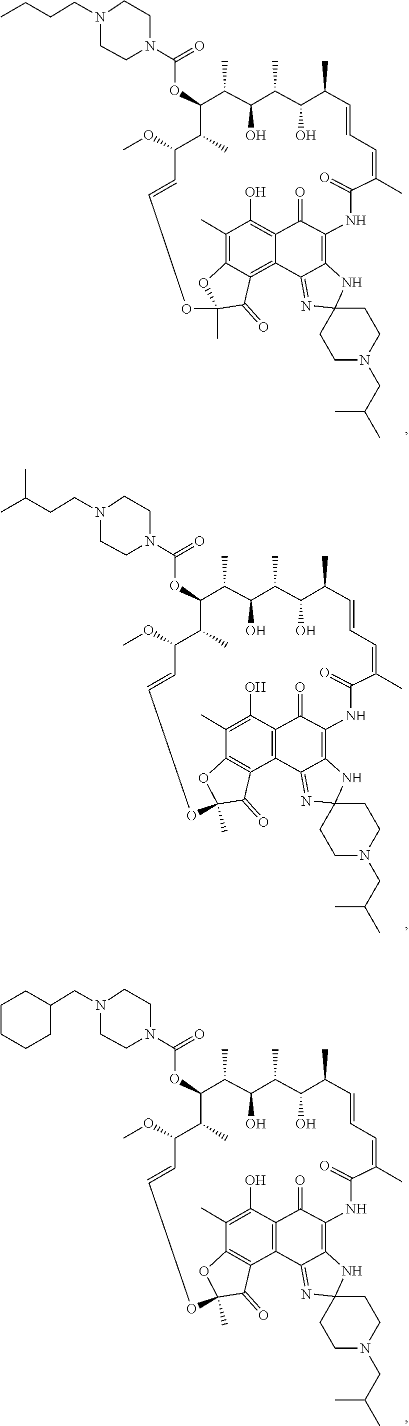 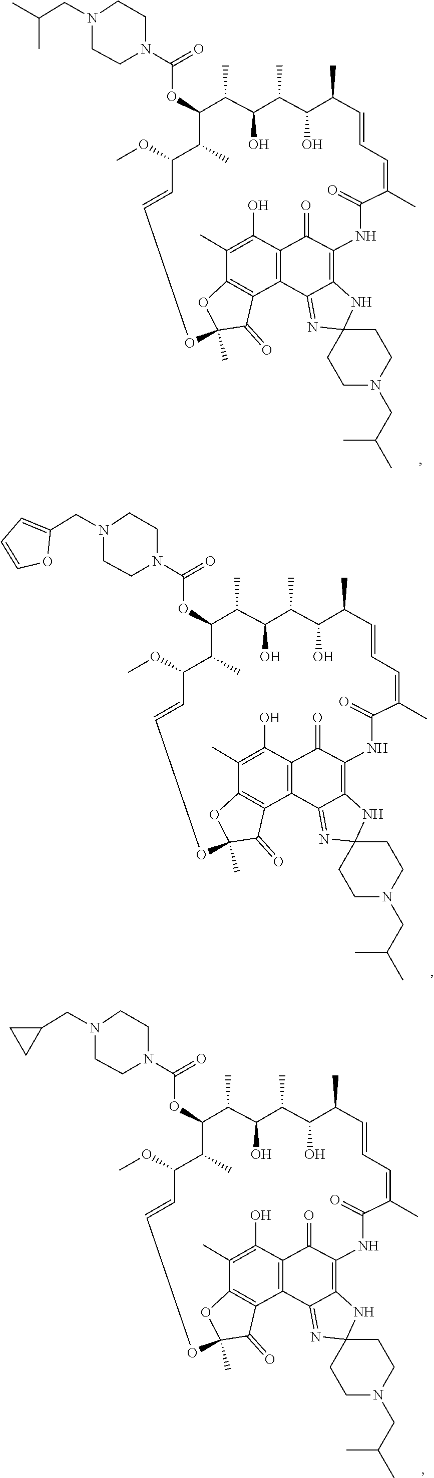 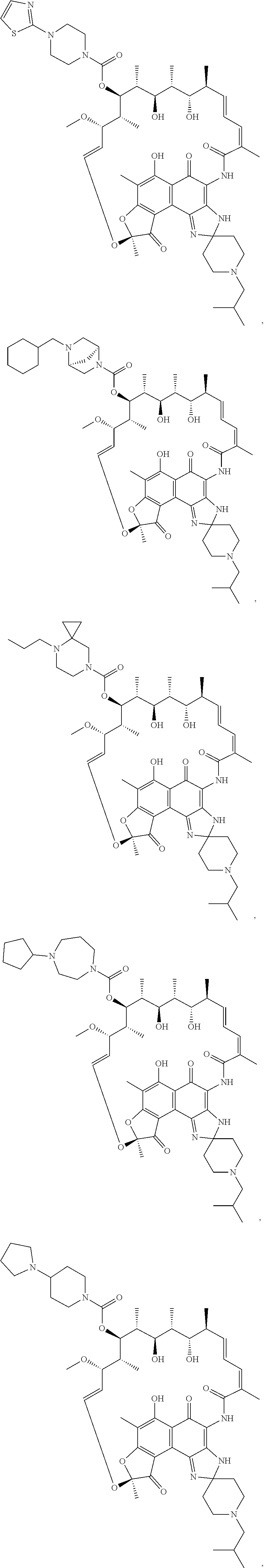 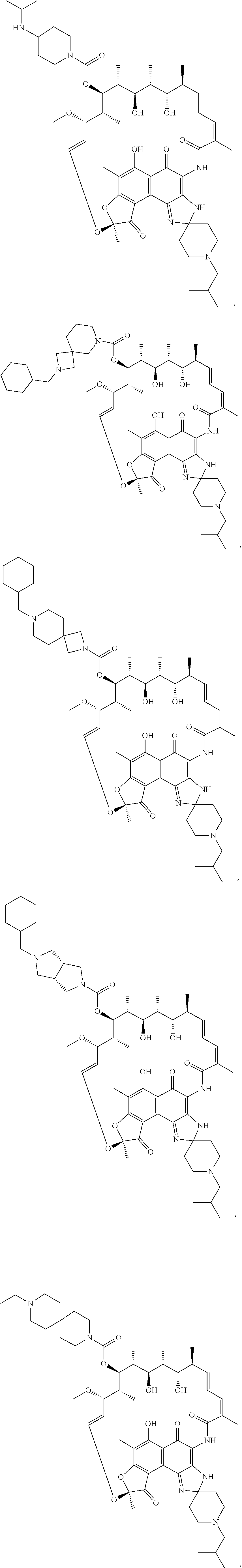 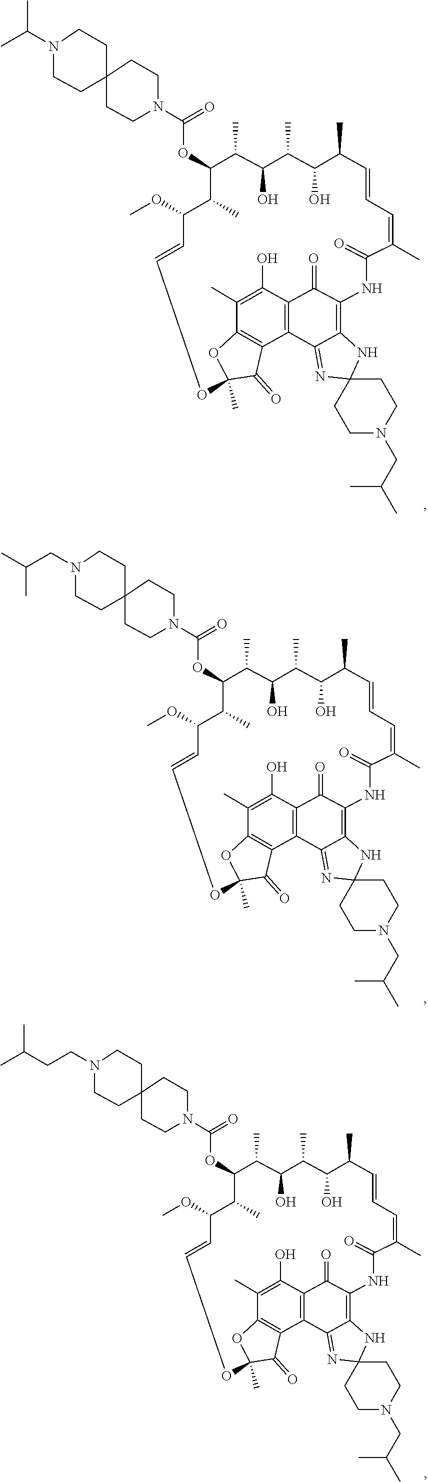 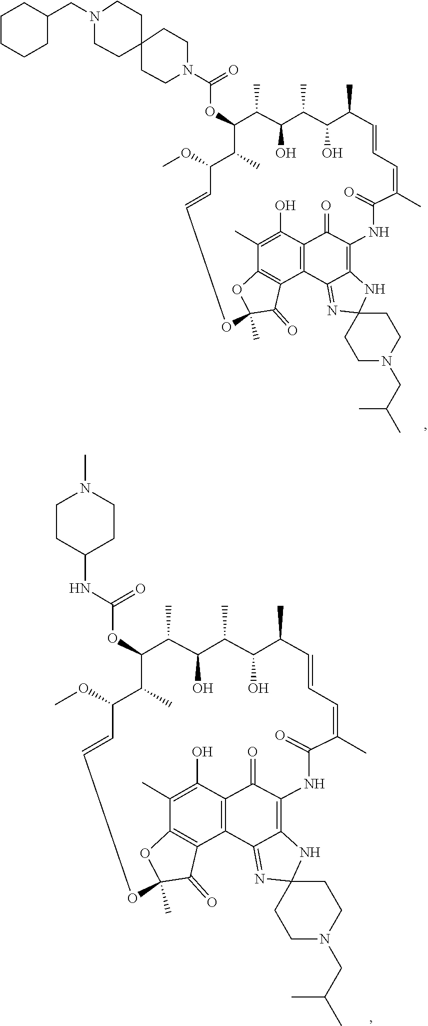 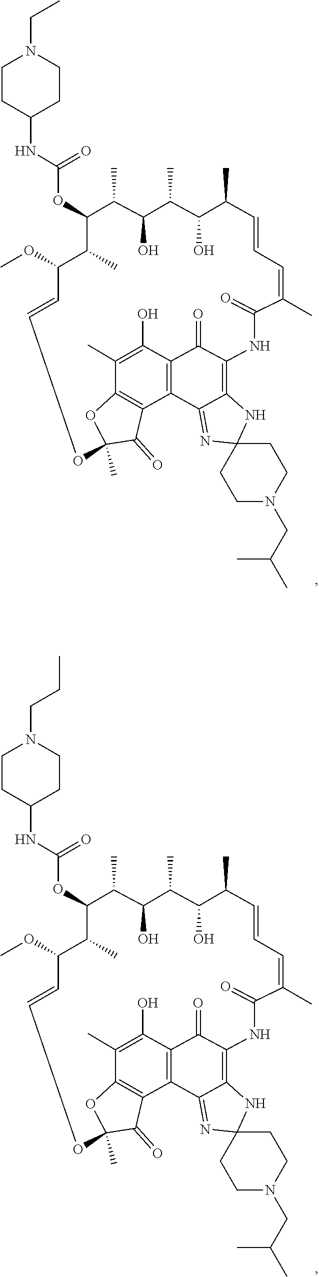 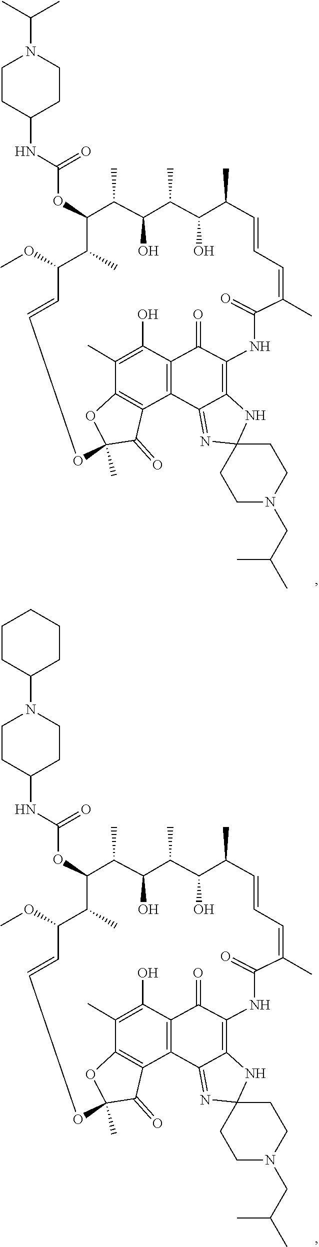 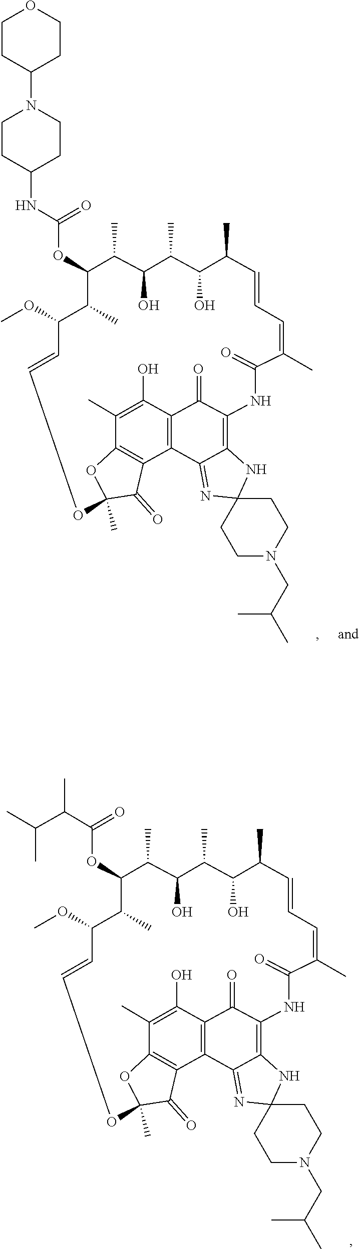
or a pharmaceutically acceptable salt thereof.

13. A pharmaceutical composition comprising at least one compound according to any of the preceding claims, or a pharmaceutically acceptable salt, tautomer, solvate or hydrate thereof, and a pharmaceutically acceptable excipient.

14. A compound according to any of the claims 1 to 12 or a pharmaceutically acceptable salt, tautomer, solvate or hydrate thereof, for use as a medicament.

15. A compound of according to any of the claims 1 to 12 or a pharmaceutically acceptable salt, tautomer, solvate or hydrate thereof, for use in a method of preventing or treating a disease in a subject, preferably wherein said disease is an infection, further preferably a bacterial infection, yet further preferably a bacterial infection caused by one or more bacterium belonging to a genus of non-tuberculous Mycobacteria, preferably M. abscessus.
